(19) United States
(12) Patent Application Publication
(10) Pub. No.: US 2012/0095512 A1
(43) Pub. Date: Apr. 19, 2012

(54) CROSS CONNECTORS

(76) Inventor: Raj Nihalani, Irvine, CA (US)

(21) Appl. No.: 13/274,233

(22) Filed: Oct. 14, 2011

Related U.S. Application Data
(63) Continuation-in-part of application No. 12/962,996, filed on Dec. 8, 2010.
Application No. 12/962,996 is a continuation-in-part of application No. 12/906,991, filed on Oct. 18, 2010.

(51) Int. Cl. A61B 17/70

(52) U.S. Cl. 606/251

(57) ABSTRACT

The present invention may provide various improvements over conventional cross connectors. For example, the present invention may provide various types of Real-X cross connectors, which may have an arch shape X-bridge that curves above the spinal bone segments of the patient. As such, the Real-X cross connectors may be more adaptive to the patient's spinal provide and provide better protect for the patient's the spinal bone segments. Moreover, the Real-X cross connectors may incorporate a complementary pivot joint configuration for smoothening the stress distribution and reducing the stress concentration around the center of the arch shape X-bridge. Advantageously, the complementary pivot joint configuration may enhance the rigidity and stability of the Real-X cross connectors.

### RELATED APPLICATIONS

This application is a continuation-in-part of application Ser. No. 12/962,996, entitled “CROSS CONNECTORS,” filed on Dec. 8, 2010, which is a continuation-in-part of application Ser. No. 12/906,991, entitled “CROSS CONNECTORS,” filed on Oct. 18, 2010. The aforementioned related applications are assigned to the assignee hereof and hereby expressly incorporated by reference herein.

### BACKGROUND

1. Field

The present invention relates generally to the field of medical devices used in posterior spinal fixation surgery, and more particularly to cross connectors.

2. Description of the Related Art

Posterior spinal fixation surgery is a common procedure for patients who suffer from severe spinal conditions, such as spinal displacement, spinal instability, spinal degeneration, and/or spinal stenosis. Among other therapeutic goals, a successful posterior spinal fixation surgery may lead to the stabilization and fusion of several spinal bone segments of a patient. During a posterior spinal fixation surgery, a spine surgeon may insert several pedicle screws into one side of several spinal bone segments of the patient to establish several anchoring points. Then, the spine surgeon may engage and secure a stabilizing rod to the several anchoring points to restrict or limit the relative movement of the spinal bone segments.

Next, this procedure may be repeated on the other side of the spinal bone segments, such that two stabilizing rods may be anchored to both sides of the spinal bone segments of the patient. To further restrict or limit the relative movement of the spinal bone segments, a connector may be used to connect the two stabilizing rods, so that the two stabilizing rods may maintain a relatively constant distance from each other. When the posterior spinal fixation surgery is completed, the operated spinal bone segments may be substantially stabilized such that they may be in condition for spinal fusion.

Conventional connectors may suffer from several drawbacks. For example, some conventional connectors may be made of flat and straight arms, such that surgeons may have a difficult time in adjusting these connectors to fit the contour the of patient's spinal bone segments. Accordingly, the implantation of these conventional connectors may require the removal of the patient's spinous process from one or more spinal bone segments because they may not be adaptive to the spinal bone structure of the patient. Moreover, most conventional connectors may not be able to protect any damaged spinal bone segment of the patient because they are can only cover a small area. Furthermore, most conventional connectors lack pre-fixation flexibility, such that they may not be adjusted to fit patients with various spinal bone widths or asymmetrical spinal bone profile.

Thus, there are needs to provide cross connectors with improved features and qualities.

### SUMMARY

The present invention may provide various improvements over conventional connectors. For example, the present invention may provide various types of Real-X cross connectors, which may have an arch shape X-bridge that curves above the spinal bone segments of the patient. As such, the Real-X cross connectors may be more adaptive to the patient's spinal bone contour and provide better protect for the patient's spinal bone segments. For another example, the present invention may provide various types of Real-O cross connectors, which may have a protection ring that may surround the patient's spinous process. Because of its protection ring, the implantation of one of the Real-O cross connectors may eliminate the need of spinous process removal. Furthermore, as provided by the present invention, the Real-O cross connector may be combined with the Real-X cross connector to form a Real-XO cross connector, which may inherit the functional benefits of both Real-X and Real-O cross connectors.

In one embodiment, the present invention may provide a cross connector for stabilizing and protecting one or more fixation levels of spinal bone segments. The cross connector may include a plurality of arms including first, second, third, and fourth arms, the first arm and the third arm aligning along a first reference plane, the second arm and the fourth arm aligning along a second reference plane intersecting the first reference plane along a pivot axis, a bottom plate centered along the pivot axis and substantially perpendicular to the first and second reference planes, a pair of bottom side walls connected to the bottom plate so as to define a bottom valley having a plurality of bottom curved sections, each of the pair of bottom side walls connected to the first arm or the third arm to form a first contiguous arc segment, a top plate snugly fitted within the bottom valley and engaging the bottom plate to provide a pivot point along the pivot axis, and a pair of top side walls connected to the top plate so as to define a top valley having a plurality of top curved sections for embracing the bottom plate, each of the pair of top side walls connected to the second arm or the fourth arm to form a second contiguous arc segment.

In another embodiment, the present invention may provide a cross connector for stabilizing and protecting one or more fixation levels of spinal bone segments. The cross connector may include a first connector including a first pair of arms and a first joint positioned between the first pair of arms, the first joint having a first platform having a first bell-shaped ridge connecting the first pair of arms to form a first contiguous arc along a first reference plane, the first bell-shaped ridge furnished with a first convex edge, and a first bracket formed on the first platform, the first bracket having a first vertical concave contour substantially parallel to the first reference plane, and a first horizontal concave contour intersecting the first vertical concave contour and substantially perpendicular to the first reference plane, a second connector including a second pair of arms and a second joint positioned between the second pair of arms, the second joint having a complementary configuration with respect to the first joint, the second joint connecting the second pair of arms to form a second contiguous arc along a second reference plane intersecting the first reference plane alone a center axis, and a pivoting means for pivoting the first connector against the second connector along the center axis, thereby allowing a limited range of angular movement between the first pair of arms and the second pair of arms.

In yet another embodiment, the present invention may include a cross connector for stabilizing and protecting one or more fixation levels of spinal bone segments. The cross connector may include a first link including a first pair of arms, a lower platform, and two upper brackets, the lower platform having two bottom bow-shaped ridges connecting the first pair of arms to form a first contiguous arc along a first reference plane, the two bottom bow-shaped ridges each furnished with a bottom convex edge, the two upper brackets positioned between the two bottom bow-shaped ridges and each having an upper ventral concave surface facing away from one of the first pair of arms, a second link including a second pair of arms, an upper platform, and two lower brackets, the upper platform having two upper bow-shaped ridges connecting the second pair of arms to form a second contiguous arc along a second reference plane intersecting the first reference plane alone a center axis, the two upper bow-shaped ridges each furnished with an upper convex edge, the two lower brackets positioned between the two upper bow-shaped ridges and each having a lower ventral concave surface facing away from one of the first pair of arms, and a pivoting member connected to the lower and upper platforms, thereby pivoting the first link against the second link along the center axis while substantially restricting a lateral movement between the first link and the second link.

### BRIEF DESCRIPTION OF THE DRAWINGS

Other systems, methods, features, and advantages of the present invention will be or will become apparent to one skilled in the art upon examination of the following figures and detailed description. It is intended that all such additional systems, methods, features, and advantages be included within this description, be within the scope of the present invention, and be protected by the accompanying claims. Component parts shown in the drawings are not necessarily to scale, and may be exaggerated to better illustrate the important features of the present invention. In the drawings, like reference numerals designate like parts throughout the different views, wherein:

FIGS. 1A-1C show various views of a Real-X cross connector according to an embodiment of the present invention;

FIGS. 1D-1G show various views of the Real-X cross connector being anchored to three spinal bone segments according to an embodiment of the present invention;

FIGS. 2A-2C show various views of a Real-X cross connector with four anchoring devices according to an embodiment of the present invention;

FIGS. 2D-2F show a top perspective view and the top views of the Real-X cross connector with four hook members being anchored to three spinal bone segments according to an embodiment of the present invention;

FIGS. 3A-3C show various views of a Real-X cross connector with four articulated rods as the connecting devices according to an embodiment of the present invention;

FIGS. 3D-3H show a top perspective view and the top views of the Real-X cross connector with four articulated rods being anchored to three spinal bone segments according to an embodiment of the present invention;

FIGS. 4A-4C show various views of a Real-X cross connector with adjustable arms according to an embodiment of the present invention;

FIGS. 4D-4F show the cross-sectional side views of several configurations of the arm length adjustable device according to various embodiments of the present invention;

FIGS. 4G-4I show various configurations of the Real-X cross connector with the adjustable arms according to various embodiments of the present invention;

FIGS. 5A-5C show various views of a fulcrum member according to an embodiment of the present invention;

FIGS. 6A-6C show various views of an alternative fulcrum member according to an embodiment of the present invention;

FIGS. 7A-7C show various views of a Real-X cross connector with two adjustable rods as the connecting devices according to an embodiment of the present invention;

FIGS. 8A-8B show a perspective view and a cross-sectional side view a Real-O cross connector (ROCC) according to an embodiment of the present invention;

FIGS. 8C-8D show a perspective view and a cross sectional side view of an alternative Real-O cross connector (ROCC) according to another embodiment of the present invention;

FIG. 8E shows a top view of the ROCC being anchored between two stabilizing rods according to an embodiment of the present invention;

FIGS. 8F-8G show the top views of the alternative ROCC being anchored between two stabilizing rods according to an embodiment of the present invention;

FIGS. 9A-9B show a perspective view and a cross-sectional side view of a Real-O cross connector with an adjustable ring according to an embodiment of the present invention;

FIGS. 10A-10H show the Real-O cross connector with rings of various shapes according to various embodiments of the present invention;

FIGS. 11A-11D show various views of a Real-XO cross connector (RXOCC) according to an embodiment of the present invention;

FIGS. 11E-11G show various configurations of the RXOCC according to various embodiments of the present invention;

FIGS. 12A-12E show various views of an alternative lockable joint member according to an embodiment of the present invention;

FIGS. 13A-13C show various views of a Real-X cross connecting pedicle screw (RXCCPS) system according to an embodiment of the present invention;

FIG. 14 shows an exploded view of a Real-X cross connector with an integrated fulcrum member according to an embodiment of the present invention;

FIG. 15 shows a top view of a semi-adjustable length Real-X cross connector with spherical joints according to an embodiment of the present invention;

FIG. 16 shows a top view of a fully adjustable Real-X cross connector with spherical joints according to an embodiment of the present invention;

FIGS. 17A-17C show various views of the joint receiving pedicle screw according to an embodiment of the present invention;

FIGS. 18A-18D show various views of the set screw according to an embodiment of the present invention;

FIGS. 19A-19C show various views of a joint receiving pedicle screw according to an embodiment of the present invention;

FIGS. 20A-20C show various views of an alternative joint receiving pedicle screw according to an embodiment of the present invention;

FIG. 21 shows a perspective view of an RXB cross connector according to a first alternative embodiment of the present invention;

FIGS. 22A-22B show a front view and a back view of the RXB cross connector according to the first alternative embodiment of the present invention;

FIGS. 23A-23B show a left side view and a front side view of the RXB cross connector according to the first alternative embodiment of the present invention;

FIG. 24 shows an exploded view of the RXB cross connector according to the first alternative embodiment of the present invention;

FIGS. 25A-25E show various views of a top link of the RXB cross connector according to the first alternative embodiment of the present invention;

FIGS. 26A-26E show various views of a bottom link of the RXB cross connector according to the first alternative embodiment of the present invention;

FIG. 27 shows a perspective view of an RXC cross connector according to a second alternative embodiment of the present invention;

FIGS. 28A-28B show a front view and a back view of the RXC cross connector according to the second alternative embodiment of the present invention;

FIGS. 29A-29B show a left side view and a front side view of the RXC cross connector according to the second alternative embodiment of the present invention;

FIG. 30 shows an exploded view of the RXC cross connector according to the second alternative embodiment of the present invention;

FIGS. 31A-31E show various views of a top link of the RXC cross connector according to the second alternative embodiment of the present invention;

FIGS. 32A-32E show various views of a bottom link of the RXC cross connector according to the second alternative embodiment of the present invention;

FIG. 33A shows a perspective view of a stress test set up for the RXB cross connector according to the first alternative embodiment of the present invention;

FIG. 33B shows a perspective view of a stress test set up for the RXC cross connector according to the second alternative embodiment of the present invention;

FIG. 34A shows a chart of a stress test result of the RXB cross connector according to the first alternative embodiment of the present invention;

FIG. 34B shows a chart of a stress test result of the RXC cross connector according to the second alternative embodiment of the present invention;

FIG. 35 shows a perspective view of a pedicle screw utilizing a spherical joint according to an embodiment of the present invention;

FIGS. 36A-36B show various views of the disassembled pedicle screw utilizing the spherical joint according to the embodiment shown in FIG. 35;

FIGS. 37A-37B show various views of the disassembled pedicle screw utilizing the spherical joint according to the embodiment shown in FIG. 35 connecting with a spherical connecting rod;

FIG. 38 shows a perspective view of a Real-X cross connector utilizing a spherical joint at each arm according to an embodiment of the present invention;

FIG. 39 shows a perspective view of the disassembled Real-X cross connector utilizing a spherical joint at each arm according to the embodiment shown in FIG. 38;

FIGS. 40A-40B show perspective views of a first connector and a second connector of the Real-X cross connector utilizing a spherical joint at each arm according to the embodiment shown in FIG. 38;

FIGS. 41A-41C show various views of spherical connecting rods and an associated set screw for connecting the spherical connecting rods to the arms of the Real-X cross connector utilizing a spherical joint at each arm;

FIG. 42 shows a perspective view of an alternative Real-X cross connector utilizing a spherical joint at each arm according to an embodiment of the present invention;

FIG. 43 shows a perspective view of a Real-X cross connector utilizing a spherical joint at a fulcrum according to an embodiment of the present invention;

FIG. 44 shows a perspective view of the disassembled Real-X cross connector utilizing a spherical joint at a fulcrum according to the embodiment shown in FIG. 43;

FIGS. 45A-45B show perspective views of a first connector and a second connector of the Real-X cross connector utilizing a spherical joint at a fulcrum according to the embodiment shown in FIG. 43;

FIGS. 46A-46B show various views of a set screw for connecting the first connector to the second connector via a spherical joint at a fulcrum of the Real-X cross connector according to an embodiment of the present invention;

FIG. 47 shows a perspective view of a spinal bridge utilizing a spherical joint but without a crossed configuration according to an embodiment of the present invention;

FIG. 48 shows a perspective view of the disassembled spinal bridge according to the embodiment shown in FIG. 47;

FIGS. 49A-49B show perspective views of a dimpled surface of a Real-X cross connector according to an embodiment of the present invention;

FIGS. 50A-50B show various views of a collapsible minimally invasive cross connector according to an embodiment of the present invention; and

FIGS. 51A-51C show various views of a geared minimally invasive cross connector according to an embodiment of the present invention.

### DETAILED DESCRIPTION

Apparatus, systems and methods that implement the embodiment of the various features of the present invention will now be described with reference to the drawings. The drawings and the associated descriptions are provided to illustrate some embodiments of the present invention and not to limit the scope of the present invention. Throughout the drawings, reference numbers are re-used to indicate correspondence between reference elements. In addition, the first digit of each reference number indicates the figure in which the element first appears.

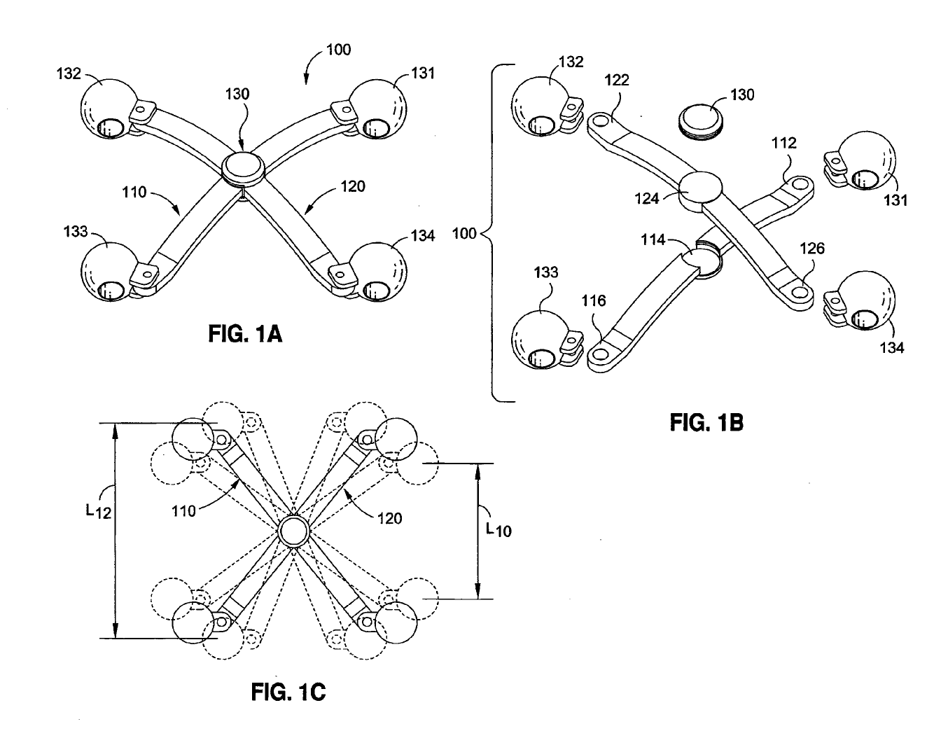

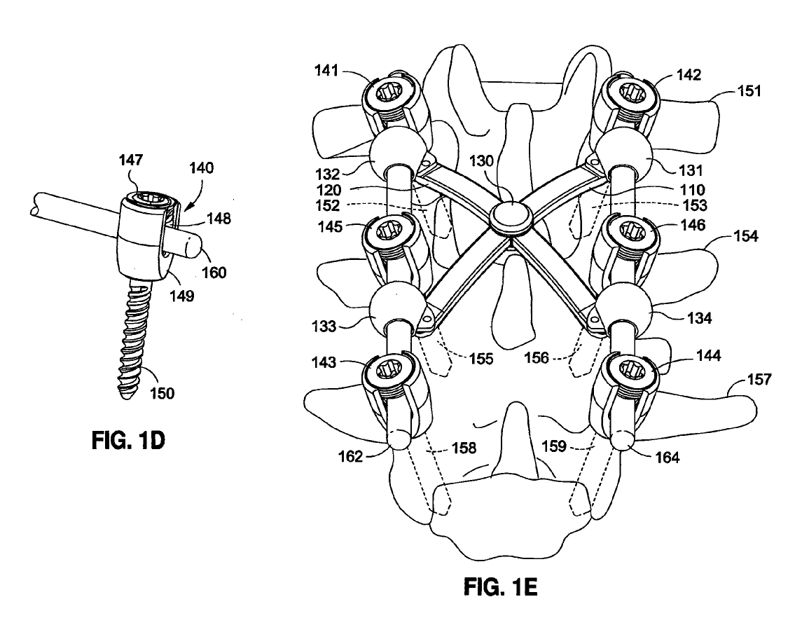

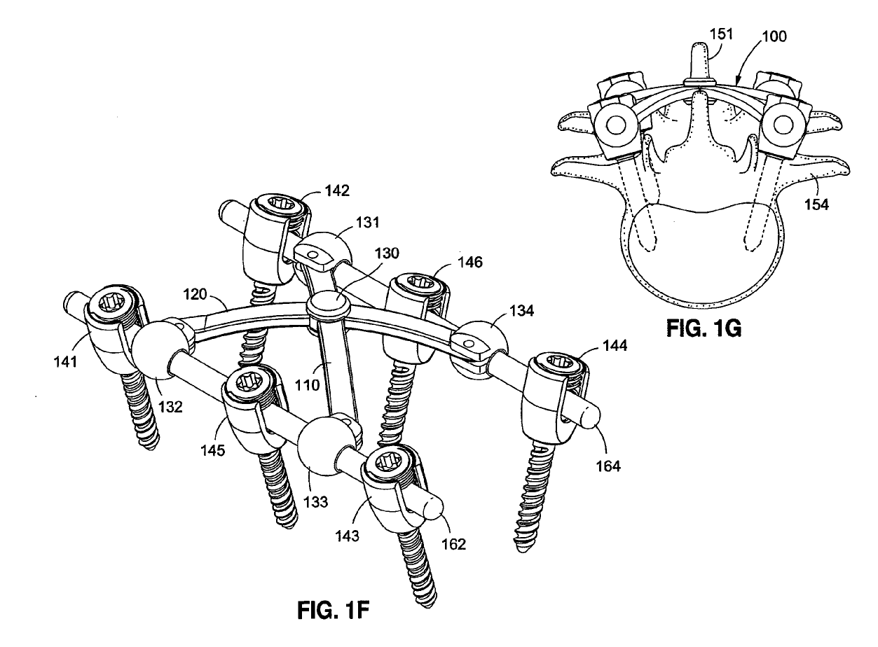

FIGS. 1A-1C show various views of a Real-X cross connector (RXCC) 100 according to an embodiment of the present invention. As shown in FIG. 1A, the RXCC 100 may include a first elongated member (first arm) 110, a second elongated member (second arm) 120, a fulcrum member 130, and four connecting devices 131, 132, 133, and 134. Generally, as shown in FIG. 1B, the first and second elongated members 110 and 120 may have first ends 112 and 122, second ends 116 and 126, and pivot segments 114 and 124.

In one embodiment of the present invention, the fulcrum member 130 may engage both the pivot segment 114 of the first elongated member 110 and the pivot segment 124 of the second elongated member 120. Consequently, as shown in FIG. 1C, the first elongated member 110 may have a range of pivotal movement with the second elongated member 120. Advantageously, the RXCC 100 may be adjusted to have a minimum width L₁₀ and a maximum width L₁₂ between the first ends 112 and 122 and/or the second ends 116 and 126. In one embodiment, the minimum width L₁₀ may be about 5 mm while the maximum width L₁₂ may be about 120 mm. In another embodiment, the minimum width L₁₀ may be about 10 mm while the maximum width L₁₂ may be about 100 mm. In yet another embodiment, the minimum width L₁₀ may be about 12 mm while the maximum width L₁₂ may be about 88 mm.

As shown in FIG. 1B, the first and second elongated members 110 and 120 may each have an arch. In one embodiment, the pivot segments 114 and 124 may form the top parts of the arch, whereas the first and second ends 112, 122, 116, and 126 may form the bottom parts of the arch. Together, the first and second elongated members 110 and 120 may form an X-shape protection bridge with a convex profile, which may fit and adapt to a posterior contour of several spinal bone segments. Advantageously, the RXCC 100 may be placed across one or more spinal bone segments for protecting a defected bone segment or a partially exposed spinal cord (not shown).

Moreover, the RXCC 100 may be equipped with the first connecting device 131, the second connecting device 132, the third connecting device 133, and the fourth connecting device 134. More specifically, the first connecting device 131 may be coupled to the first end 112 of the first elongated member 110, the second connecting device 132 may be coupled to the first end 122 of the second elongated member 120, the third connecting device 133 may be coupled to the second end 116 of the first elongated member 110, and the fourth connecting device 134 may be coupled to the second end 126 of the second elongated member 120.

The four connecting devices 131, 132, 133, and 134 may be used for connecting the RXCC 100 to a group of pedicle screws or two stabilizing rods, both of which may be anchored to one or more spinal bone segments. As such, the RXCC 100 may substantially reduce or minimize the relative movement among the pedicle screws or among the two stabilizing rods. Advantageously, the RXCC 100 may provide extra support and stability to one or more spinal bone segments by virtue of connecting to the group of pedicle screws or the two stabilizing rods.

FIGS. 1D-1F show various views of the Real-X cross connector (RXCC) 100 being anchored to three spinal bone segments 151, 154, and 157 according to an embodiment of the present invention. Generally, as shown in FIG. 1D, a pedicle screw 140 may include a set screw 147, a threaded shaft 150, and a base member 149. More specifically, the threaded shaft 150 may be used for drilling into a spinal bone segment, the base member 149 may have a pair of receiving ports 148 for receiving a stabilizing rod 160, and the set screw 147 may be used for securing the stabilizing rod 160 to the base member 149.

Referring to FIG. 1E, six pedicle screws 141, 142, 143, 144, 145, and 146 may be used to anchor the spinal bone segments 151, 154, 157. For example, the pedicle screws 141 and 142 may be drilled into the spinal bone segments 151 via the left pedicle 152 and the right pedicle 153 respectively. For another example, the pedicle screws 145 and 146 may be drilled into the spinal bone segments 154 via the left pedicle 155 and the right pedicle 156 respectively. For yet another example, the pedicle screws 143 and 144 may be drilled into the spinal bone segments 157 via the left pedicle 158 and the right pedicle 159 respectively.

After the anchoring process, the first stabilizing rod 162 may be received and secured by the anchored pedicle screws 141, 143, and 145, while the second stabilizing rod 164 may be received and secured by the anchored pedicle screws 142, 144, and 146. Accordingly, the first stabilizing rod 162 may be anchored to the spinal bone segments 151, 154, and 157 along a left pedicle line defined by the left pedicles 152, 155, and 158, and the second stabilizing rod 164 may be anchored to the spinal bone segments 151, 154, and 157 along a right pedicle line defined by the right pedicles 153, 156, and 159. Depending on the particular group of spinal bone segments being operated on, the left and right pedicle lines may be parallel to each other or they may be angularly positioned.

Next, the RXCC 100 may be placed over the spinal bone segments 151, 154, and 157. For example, as shown in FIGS. 1E and 1F, the first connecting member 131 may connect the first end 112 of the first elongated member 110 to the second stabilizing rod 164 between the pedicle screws 142 and 146, the second connecting member 132 may connect the first end 122 of the second elongated member 120 to the first stabilizing rod 162 between the pedicle screws 141 and 145, the third connecting member 133 may connect the second end 126 of the second elongated member 120 to the second stabilizing rod 164 between the pedicle screws 146 and 144, and the fourth connecting member 134 may connect the second end 116 of the first elongated member 110 to the first stabilizing rod 161 between the pedicle screws 145 and 143.

After the RXCC 100 is connected to the first and second stabilizing rods 162 and 164, the RXCC 100 may form the X-shape protection bridge over and across one or more spinal bone segments. In one configuration, the RXCC 100 may form the X-shape protection bridge for protecting the spinal bone segment 154. In another configuration, the RXCC 100 may form the X-shape protection bridge for protecting the spinal bone segment 151. In yet another configuration, the RXCC 100 may form the X-shape protection bridge for protecting the spinal bone segment 157.

Advantageously, because the first and second elongated members 110 and 120 may have the range of relative pivotal movement as shown in FIG. 1C, the RXCC 100 may be adjusted to adapt to spinal bone segments with various widths. Moreover, as shown in FIGS. 1F and 1G, the convex profile of the X-shape protection bridge may arch over the bone protrusions of one or more spinal bone segments, such that no additional surgical procedure may be required to remove any of these bone protrusions. Furthermore, the RXCC 100 may further stabilize the spinal bone segments 151, 154 and 157 by restricting and/or limiting a relative movement between the first and second stabilizing rods 162 and 164.

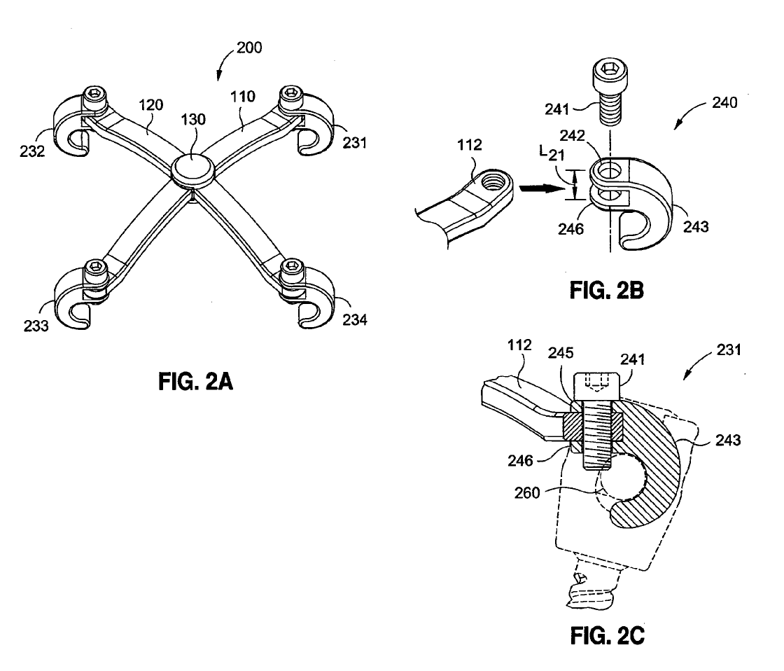

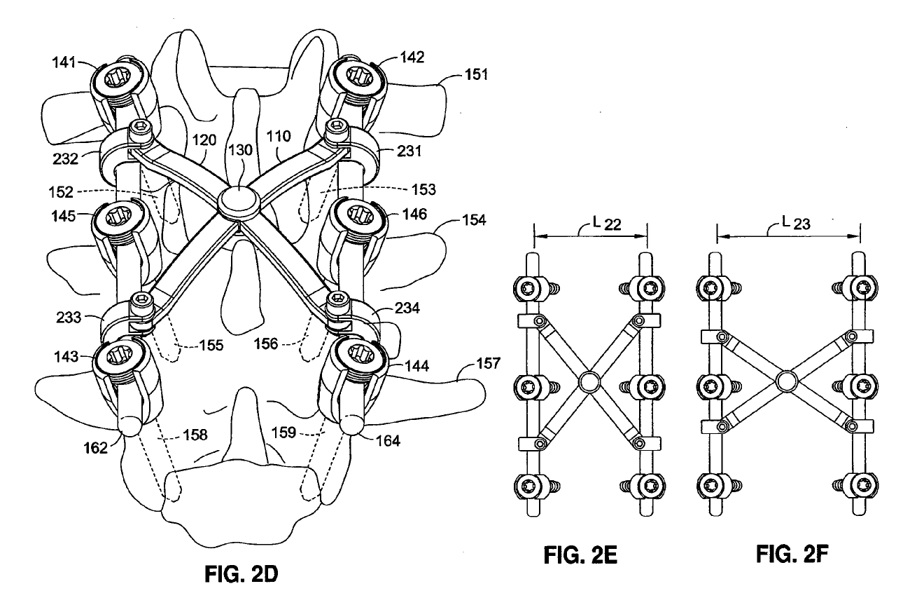

According to an embodiment of the present invention, FIGS. 2A-2C show various views of a Real-X cross connector (RXCC) 200 with four anchoring devices 231, 232, 233, and 234. The RXCC 200 may be similar to the RXCC 100 in several aspects. For example, the RXCC 200 may include the first elongated member (first arm) 110, the second elongated member (second arm) 120, and the fulcrum member 130. For another example, the first and second elongated members 110 and 120 may have first ends 112 and 122, second ends 116 and 126, and pivot segments 114 and 124. For yet another example, RXCC 200 may form an X-shape protection bridge, which may have similar structural and functional features as the X-shape protection bridge of the RXCC 100.

Despite these similarities, the RXCC 200 may be different from the RXCC 100 in at least one embodiment. For example, the RXCC 200 may incorporate four anchoring devices 231, 232, 233, and 234 to perform the functions of the connecting devices 131, 132, 133, and 134 of the RXCC 100 as shown in FIGS. 1A-1F. According to an embodiment of the present invention, the four anchoring devices 231, 232, 233, and 234 may share the structural and functional features of an anchoring device 240 as shown in FIG. 2B.

Generally, the anchoring device 240 may include a locking screw 241, a joint member 242, and a hook member 243. More specifically, the joint member 242 may be attached to the hook member 243 while the locking screw 241 may be a separate structure. The joint member 242 may have a first disc member 245, a second disc member 246, and a space defined therebetween. In order to properly receive one of the first ends 112 or 122 or one of the second ends 116 or 126, the space may have a height L₂₁, which may be slightly greater than the thickness of each of the first and second ends 112, 122, 116, and 126. Moreover, in order to properly receive the locking screw 241, both the first and second discs 245 and 246 may each have an opening with a diameter slightly greater than a diameter of the locking screw 241.

Referring to FIG. 2C, which shows the operation of the anchoring device 231, the first end 112 of the first elongated member 110 may be inserted into the space between the first and second disc members 245 and 246 of the joint member 242, and the hook member 243 may engage a segment of a stabilizing rod 260. Next, the locking screw 241 may penetrate the first and second disc members 245 and 246 as well as the first end 112 received therebetween. Consequentially, the first end 112 may be secured to the anchoring device 231 and it may freely rotate about the locking screw 241.

In order to limit the movement of the first end 112 relative to the anchoring device 231, the locking screw 241 may fully engage the first and second disc members 245 and 246. The locking screw 241 may cooperate with the first and second disc members 245 and 246 to assert a pair of vertical forces against the top and bottom surfaces of the first end 112. Accordingly, the friction between the joint member 242 and the first end 112 may increase substantially, and the relative movement of the first end 112 may be locked at a particular angular position in relative to the hook member 243.

The above assembling procedures may be repeated for the first end 122 of the second elongated member 120, the second end 116 of the first elongated member 110, and the second end 126 of the second elongated member 120. Accordingly, the first anchoring device 231 may be coupled to the first end 112, the second anchoring device 232 may be coupled to the first end 122, the third anchoring device 233 may be coupled to the second end 116, and the fourth anchoring device 234 may be coupled to the second end 126.

After the initial assembling process, the hook member 243 may be used to engage a segment of the stabilizing rod 260. When the anchoring device is properly positioned, the locking screw 241 may be driven further to contact the segment of the stabilizing rod 260. In one embodiment of the present invention, the locking screw 241 may assert a compression force against a top part of the stabilizing rod 260, which may redirect the compression force against a bottom section of the hook member 243. As a result, the bottom section of the hook member 243 may react to the compression force and produce a reaction force, which may be asserted against a bottom part of the stabilizing rod 260. Accordingly, the compression force may cooperate with the reaction force to secure the segment of stabilizing rod 260 within the hook member 243.

FIG. 2D shows a top perspective view of the RXCC 200 anchored to three spinal bone segments 151, 154, and 157 via the pedicle screws 141, 142, 143, 144, 145, and 146 and the stabilizing rods 162 and 164. Generally, the pedicle screws 141, 142, 143, 144, 145, and 146 and the stabilizing rods 162 and 164 may be first anchored to the left and right pedicles of the spinal bone segment 151, 154, and 157 as discussed in FIGS. 1E and 1F. Like the RXCC 100, the RXCC 200 may form the X-shape protection bridge above and across the spinal bone segment 151, 154, or 157.

For example, to form the X-shape protection bridge above and across the spinal bone segment 154, the anchoring device 231 may engage the first stabilizing rod 162 between the pedicle screws 141 and 145, the anchoring device 234 may engage first stabilizing rod 162 between the pedicle screws 145 and 143, the anchoring device 232 may engage the second stabilizing rod 164 between the pedicle screws 142 and 146, and the anchoring device 233 may engage the second stabilizing rod 164 between the pedicle screws 146 and 144.

At this stage, the respective locking screws 241 may be free from contacting the first and second stabilizing rods 162 and 164, such that the RXCC 200 may still be free to slide along the first and second stabilizing rods 162 and 164. Advantageously, the X-shape protection bridge may be conveniently maneuvered to cover an area which may need to be protected. After the X-shape protection bridge is properly positioned, the respective locking screws 241 may be applied to secure the first and second rods 162 and 164 to the RXCC 200. Consequentially, the RXCC 200 may be anchored to the first and second rods 162 and 164 via the anchoring devices 231, 232, 233, and 234. At this stage, the RXCC 200 may remain relatively stationary with respect to the first and second stabilizing rods 162 and 164, the pedicle screws 141, 142, 143, 144, 145, and 146, and the spinal bone segments 151, 154, and 157.

As shown in FIGS. 2E and 2F, the RXCC 200 may be adjusted to adapt to spinal bone segments with various width. In one configuration, the RXCC 200 may be adjusted to reduce the distance between the first ends 112 and 122 or between the second ends 116 and 126 if the spinal bone segments have a narrow width L₂₂. Accordingly, the first and second anchoring devices 231 and 232 may be positioned closer to the pedicle screws 141 and 142, while the third and fourth anchoring devices 233 and 234 may be positioned closer to the pedicle screws 143 and 144. In another configuration, the RXCC 200 may be adjusted to increase the distance between the first ends 112 and 122 or between the second ends 116 and 126 if the spinal bone segments have a wide width L₂₃. Accordingly, the first and second anchoring devices 231 and 232 may be positioned farther away from the pedicle screws 141 and 142, while the third and fourth anchoring devices 233 and 234 may be positioned farther away from the pedicle screws 143 and 144.

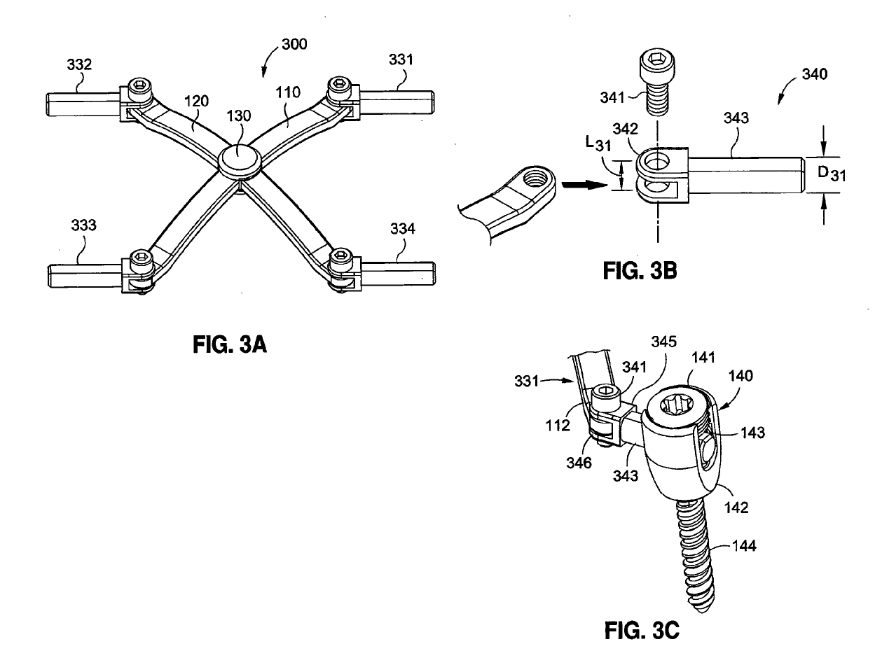

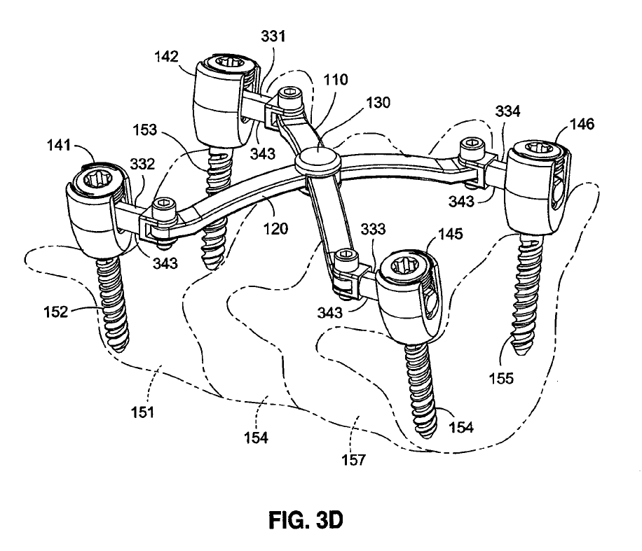

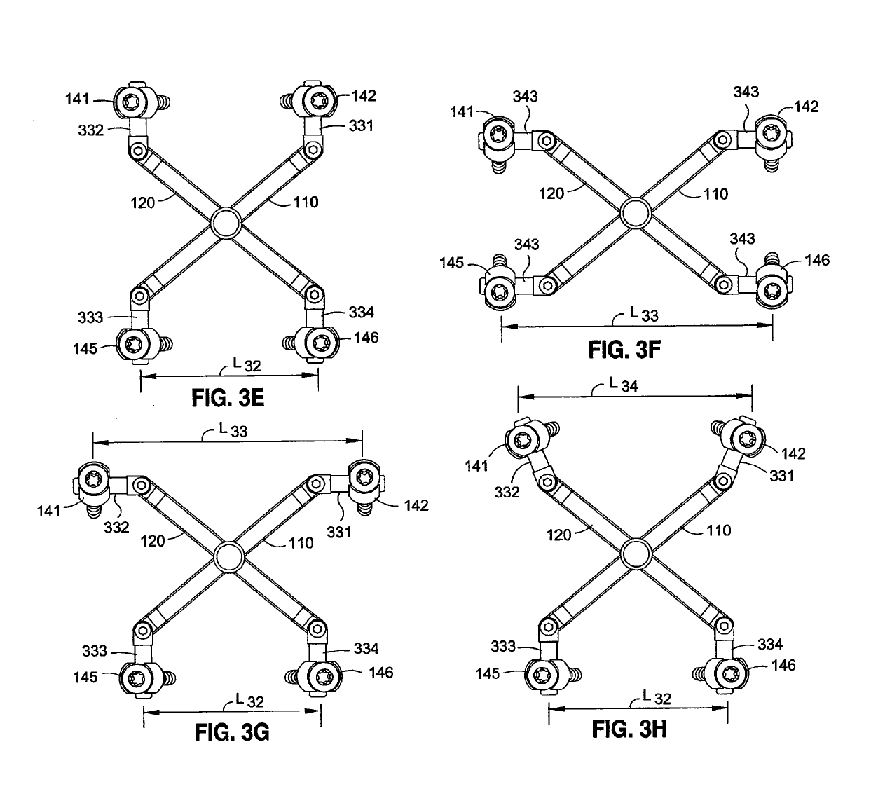

FIGS. 3A-3C show various views of a Real-X cross connector (RXCC) 300 with four articulated rods 331, 332, 333, and 334. The RXCC 300 may be similar to the RXCC 100 in several aspects. For example, the RXCC 300 may include the first elongated member (first arm) 110, the second elongated member (second arm) 120, and the fulcrum member 130. For another example, the first and second elongated members 110 and 120 may have first ends 112 and 122, second ends 116 and 126, and pivot segments 114 and 124. For yet another example, the RXCC 300 may form an X-shape protection bridge, which may have similar structural and functional features as the X-shape protection bridge formed by the RXCC 100.

Despite these similarities, the RXCC 300 may be different from the RXCC 100 in at least one aspect. For example, the RXCC 300 may incorporate four articulated rods 331, 332, 333, and 334 to perform the functions of the connecting devices 131, 132, 133, and 134 of the RXCC 100 as shown in FIGS. 1A-1F. The four articulated rods 331, 332, 333, and 334 may share the structural and functional features of an articulated rod 340 as shown in FIG. 3B.

Generally, the articulated rod 340 may include a locking screw 341, a joint member 342, and a rod member 343. More specifically, the joint member 342 may be attached to the rod member 343 while the locking screw 341 may be a separate structure. The joint member 342 may have a first disc member 345, a second disc member 346, and a space defined therebetween. In order to properly receive one of the first ends 112 or 122 or one of the second ends 116 or 126, the space may have a height L₃₁ slightly greater than the thickness of each of the first and second ends 112, 122, 116, and 126. Moreover, in order to properly receive the locking screw 341, both the first and second discs 345 and 346 may each have an opening with a diameter slightly greater than a diameter of the locking screw 341.

Referring to FIG. 3C, which shows the operation of the articulated rod 331, the first end 112 of the first elongated member 110 may be inserted into the space between the first and second disc members 345 and 346 of the joint member 342, and the rod member 343 may be secured by the pedicle screw 140. Next, the locking screw 341 may penetrate the first and second disc members 345 and 346 as well as the first end 112 positioned therebetween. Consequentially, the first end 112 may be secured to the articulated rod 331 and it may freely rotate about the locking screw 341.

In order to limit the movement of the first end 112 in relative the anchoring device 331, the locking screw 341 may fully engage the first and second disc members 345 and 346. The locking screw 341 may cooperate with the first and second disc members 345 and 346 to assert a pair of vertical forces against the surfaces of the first end 112. As such, the friction between the first and second disc members 345 and 346 and the first end 312 may increase significantly, and the relative movement of the first end 112 may thus be substantially reduced or limited.

The above assembling procedures may be repeated for the first end 122 of the second elongated member 120, the second end 116 of the first elongated member 110, and the second end 126 of the second elongated member 120. Accordingly, the first articulated rod 331 may be coupled to the first end 112, the second articulated rod 332 may be coupled to the first end 122, the third articulated rod 333 may be coupled to the second end 116, and the fourth articulated rod 334 may be coupled to the second end 126.

After the initial assembling process, the rod member 343 may be received by and secured to the pedicle screw 140, which may include components as previously shown in FIG. 1D. For example, the pedicle screw 140 may have the set screw 147, the base member 149 with the pair of receiving ports 148, and the threaded shaft 150 for drilling the spinal bone segment. Initially, the rod member 343 may be inserted into the receiving ports 148 of the pedicle screw 140. When coupled to the base member 149, the set screw 147 may apply a compression force against a top part of the rod member 343, which may redirect the compression force to the base member 149. In reacting to the compression force, the base member 149 may assert a reaction force against a bottom part of the rod member 343. As such, the reaction force may cooperate with the compression force to secure a segment of the rod member 343 to the pedicle screw 140.

The rod member 343 may have similar structural and physical properties as the conventional stabilizing rods 162 and 164 as previously shown and discussed in FIGS. 1D-1F and in FIGS. 2D-2F. Accordingly, the rod member 343 may be made of a similar material as the conventional stabilizing rods 162 and 164, and it may have a diameter D₃₁ similar to those of the conventional stabilizing rods 162 and 164. Nevertheless, the rod member 343 may be substantially shorter than the convention stabilizing rods 162 and 164 because it may only be required to extend for a relatively shorter distance. Moreover, the rod member 343 may have a flat top surface and a flat bottom surface, such that it may be secured by the pedicle screw 140 more efficiently.

FIG. 3D shows a top perspective view of the RXCC 300 anchored to three spinal bone segments 151, 154, and 157 via the pedicle screws 141, 142, 143, and 144. According to an embodiment of the present invention, the RXCC 300, when equipped with the several articulated rods 331, 332, 333, and 334, may provide similar functions as the conventional stabilizing rods 162 and 164 as previously shown in FIGS. 1A-1F and 2A-2F. For example, the first and second elongated members 110 and 120 may substantially reduce the relative movement among the spinal bone segments 151, 154, and 157 when the articulated rods 331, 331, 333, and 334 are properly anchored to the spinal bone segments 151 and 157 via the pedicle screws 141, 142, 143, and 144. Because the RXCC 300 may extend vertically and horizontally, it may provide both vertical and horizontal stabilizations to the spinal bone segments 151, 154, and 157. Advantageously, this bidirectional stabilization substantially improves the unidirectional stabilization provided by the conventional stabilizing rods 162 and 164 because it may better address the horizontal instability among several spinal bone segments.

Moreover, the RXCC 300 may obviate the need for applying the pedicle screws 145 and 146 to the spinal bone segment 154. Furthermore, the RXCC 300 may be applied to two or more fixation levels of spinal bone segments. Accordingly, the RXCC 300 may reduce the number of implantable devices and the number of procedures for installing these implantable devices. Advantageously, using the RXCC 300 may help reduce the cost and time for performing posterior spinal surgery, thereby rendering it more affordable for the patients and more efficient for the surgeons.

FIGS. 3E-3H show various configurations of the RXCC 300 according to various embodiments of the present invention. Similar to the RXCC 100 and the RXCC 200, the RXCC 300 may be adjustable to adapt to spinal bone segments with various widths. Moreover, the extra length and maneuverability provided by the articulated rods 331, 332, 333, and 334 may allow the RXCC 300 to have a wider range of adjustment.

In one embodiment, for example, the RXCC 300 may be adjusted to adapt to the spinal bone segments with a small width L₃₂ as shown in FIG. 3E. In another embodiment, for example, the RXCC 300 may be adjusted to adapt to the spinal bone segments with a large width L₃₃ as shown in FIG. 3F. In another embodiment, for example, the RXCC 300 may be adjusted to adapt to the spinal bone segments with a large top width L₃₃ but a small bottom width L₃₂ as shown in FIG. 3G. Particularly, the rod members 343 of the first and second articulated rods 331 and 332 may be positioned horizontally while the rod members 343 of the third and fourth articulated rods 333 and 334 may be positioned vertically. In yet another embodiment, for example, the RXCC 300 may be adjusted to adapt to the spinal bone segments with a medium top width L₃₄ and a small bottom width L₃₂ as shown in FIG. 3H. Particularly, the rod members 343 of the first and second articulated rods 331 and 332 may positioned diagonally while the third and fourth articulated rods 333 and 334 may be positioned vertically.

Besides the configurations as shown in FIGS. 3E-3F, the RXCC 300 may be adjusted to adapt to a wide range of symmetrical spinal bone segments as well as asymmetrical spinal bone segments. The rod members 343 may be highly maneuverable about the respective joint members 342, and thus, they can be configured to turn in any planar direction before they are firmly secured by the respective pedicle screws 140. Advantageously, the RXCC 300 may provide a dynamic range of configurations, which may be more adjustable and adaptable than the configurations provided by conventional cross connectors and the conventional stabilizing rods.

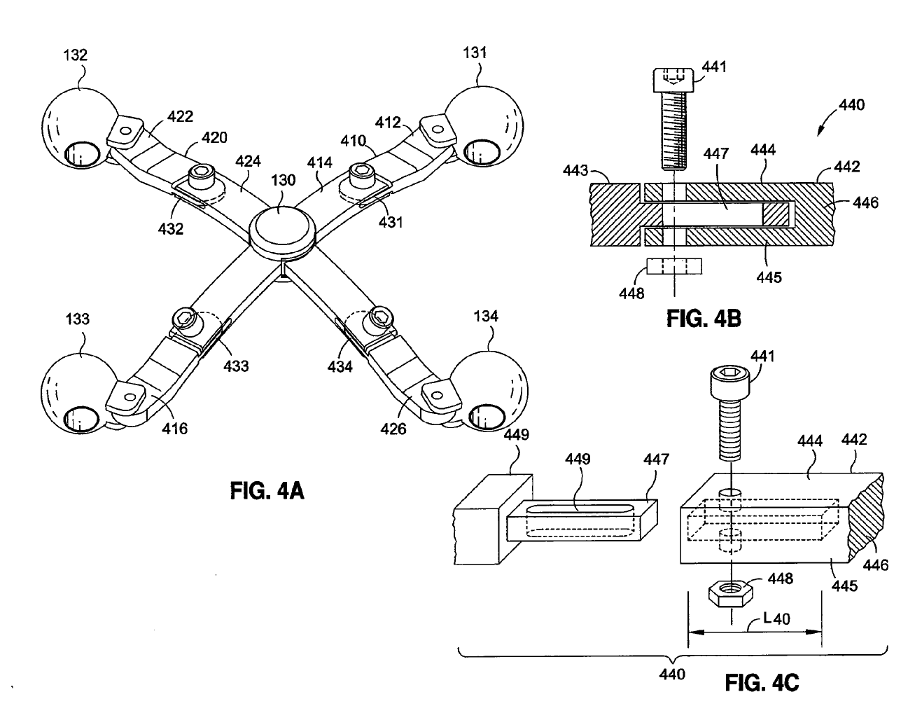

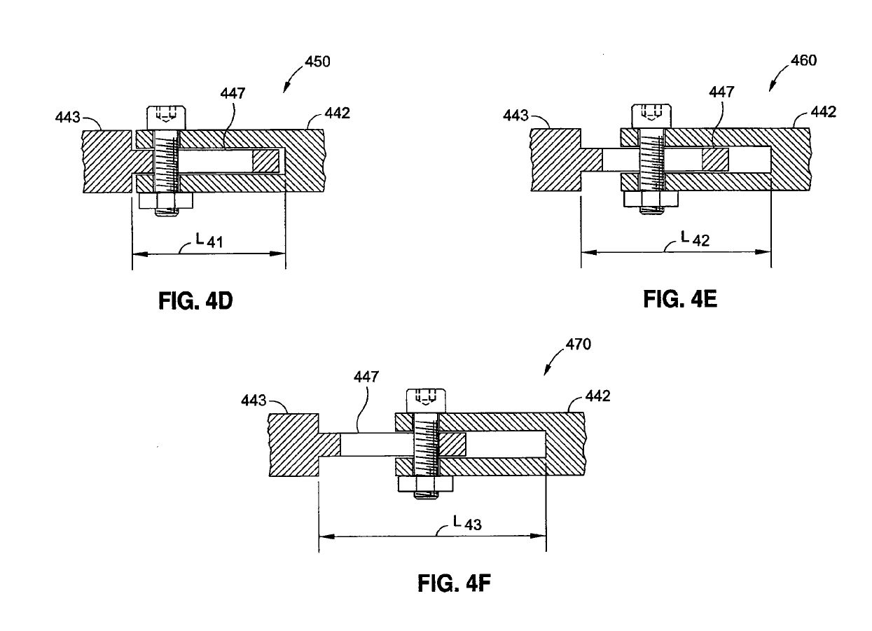

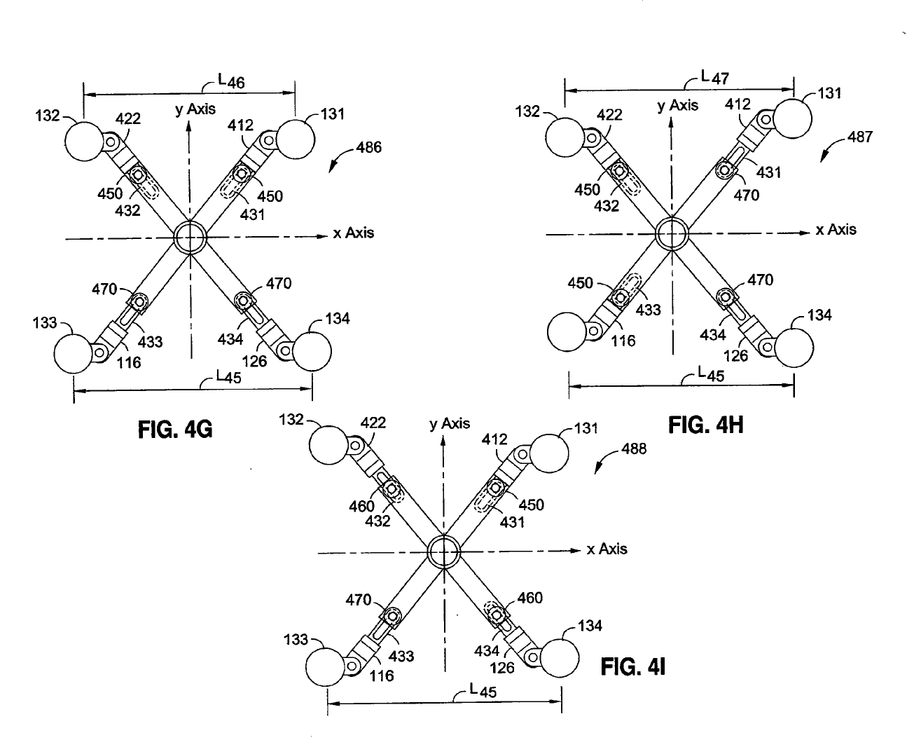

The discussion now turns to arm length adjusting feature of the Real-X cross connector. FIGS. 4A-4C show various views of a Real-X cross connector (RXCC) 400 with adjustable arms 410 and 420 according to an embodiment of the present invention. The RXCC 400 may be similar to the RXCC 100 in several aspects.

For example, the RXCC 400 may include a first elongated member (first arm) 410, a second elongated member (second arm) 420, the fulcrum member 130, and four connecting devices 131, 132, 133, and 134. The four connecting devices 131, 132, 133, and 134 may be implemented by the anchoring device 240 as shown in FIG. 2B, the articulated rod 340 as shown in FIG. 3B, or any other connecting devices, as long as they may connect the RXCC 400, directly or indirectly, to a set of readily anchored pedicle screws.

For another example, the first and second elongated members 410 and 420 may have first ends 412 and 422, second ends 416 and 426, and pivot segments 414 and 424. For another example, the fulcrum member 130 may engage and pivot the pivot segments 414 and 424, such that the first and second elongated members 410 and 420 may have a relative pivotal movement about the fulcrum member 130.

For yet another example, RXCC 400 may form an X-shape protection bridge, which may have similar structural and functional features as the X-shape protection bridge formed by the RXCC 100.

Despite these similarities, the RXCC 400 may be different from the RXCC 100 in at least one aspect. For example, the RXCC 400 may incorporate four arm length adjusting devices (ALADs) 431, 432, 433, and 434 to allow the first and second elongated members 410 and 420 to extend and/or retract their respective length. According to an embodiment of the present invention, the four ALADs 431, 432, 433, and 434 may share the structural and functional features of an ALAD 440 as shown in FIG. 4B-4C.

Generally, the ALAD 440 may include a locking screw 441, a nut member 448, a female member 442, and a male member 443. The female member 442 may be a receiving structure with a hollow core. As such, the female member 442 may include a top plate 444, a bottom plate 445 and a side wall 446. The side wall 446 may connect the top and bottom plates 444 and 445, which may define an opening and a space for receiving the male member 443. The male member 443 may have an insertion member 447 for inserting into the space of the female member 442.

In one embodiment, the female member 442 may be coupled to an end of the RXCC 400, which may be one of the first or second ends 112, 122, 116, or 126, while the male member 443 may be coupled to the pivot segment 414 or 424. In another embodiment, the male member 443 may be coupled to an end of the RXCC 400, which may be one of the first or second ends 112, 122, 116, or 126, while the female member 442 may be coupled to the pivot segment 414 or 424.

Generally, the insertion member 447 may slide into or outside of the space of the female member 442 before the locking mechanism is triggered. In one embodiment, the insertion member 447 and the space may each have a length L₄₀, which may range, for example, from 2 mm to about 20 mm. As such, the ALAD 440 may have a retracted length which may range, for example, from about 2 mm to about 20 mm, as well as an extended length which may range, for example, from about 4 mm to about 40 mm.

After the female member 442 and the male member 443 are properly adjusted to achieve a desirable arm length, the locking mechanism may be triggered. Generally, the locking mechanism may be actuated by a coupling between the locking screw 441 and the nut member 448 or by any other methods that may affix the insertion member 447 within the space of the female member 442. As shown in FIG. 4C, the top and bottom plates 444 and 445 of the female member 442 may each have a penetration port for receiving the locking screw 441, and the insertion member 447 may have a narrow slit 449 for allowing the passage of the locking screw 441. In one embodiment, the locking screw 441 may pass through the opening of the top plate 444, then the narrow slit 449, and then the opening of the bottom plate 445.

After the locking screw 441 successfully penetrates the top plate 444, the insertion member 447 and the bottom plate 445, the nut member 448 may be coupled to the locking screw 441. Accordingly, a bolt of the locking screw 441 and the nut member 448 may apply a pair of compression forces against the top and bottom plates 444 and 445 respectively. The top and bottom plates 444 and 445 may then convert the pair of compression forces to a pair of frictional forces against the surfaces of the insertion member 447. As the pair of frictional forces increase, the insertion member 447 may become less free to slide along the space of the female member 442, and eventually, the insertion member 447 may be locked at a particular position.

FIGS. 4D-4F show the cross-sectional side views of several configurations of the ALAD 440 according to various embodiments of the present invention. As shown in FIG. 4D, the ALAD 440 may have a full retraction configuration, in which the insertion member 447 may be substantially inside of the space of the female member 442. As such, the ALAD 440 may have a fully retracted length L₄₁, which may be substantially the same as the length of the insertion member L₄₀. As shown in FIG. 4E, the ALAD 440 may have a partial extension configuration, in which the insertion member 447 may be partially inside of the space of the female member 442. As such, the ALAD 440 may have a partial extended length L₄₂, which may be greater than the fully retracted length L₄₁. As shown in FIG. 4F, the ALAD 440 may have a full extension configuration, in which the insertion member 447 may be substantially outside of the space of the female member 442. As such, the ALAD 440 may have a fully extended length L₄₃, which may be greater than the partial extended length L₄₂.

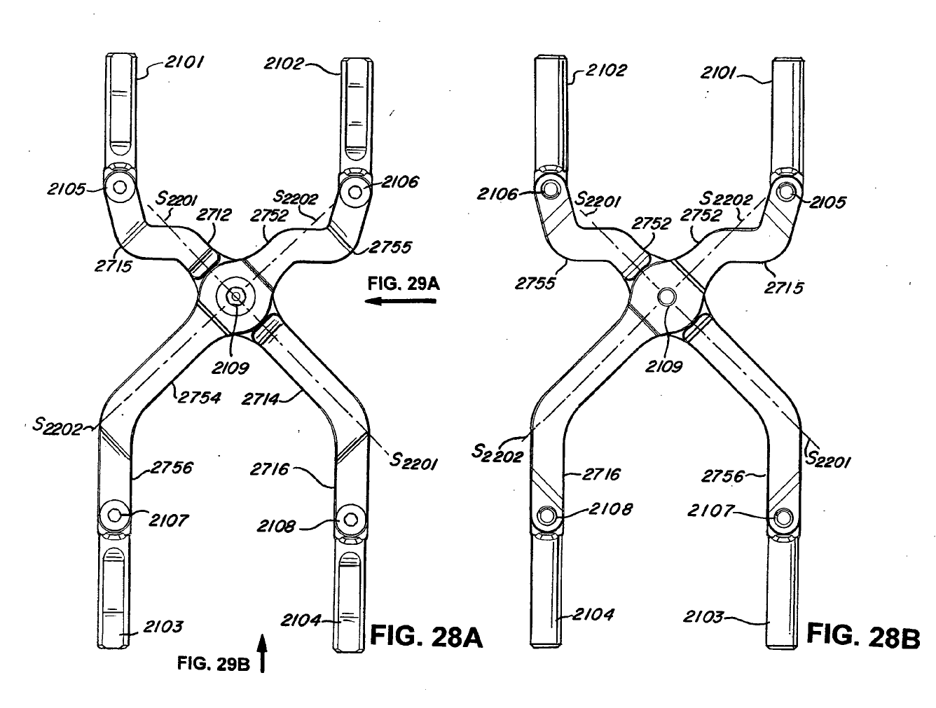

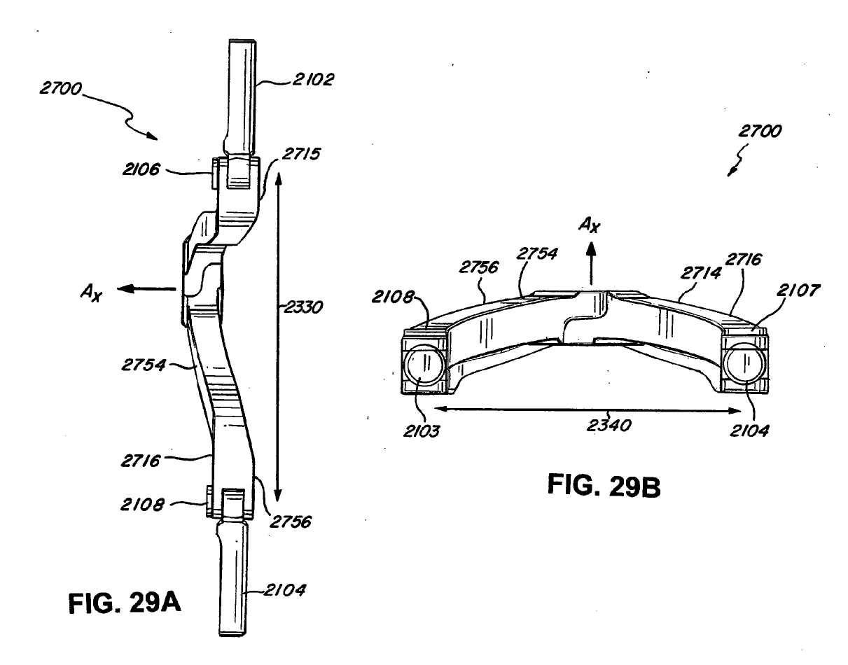

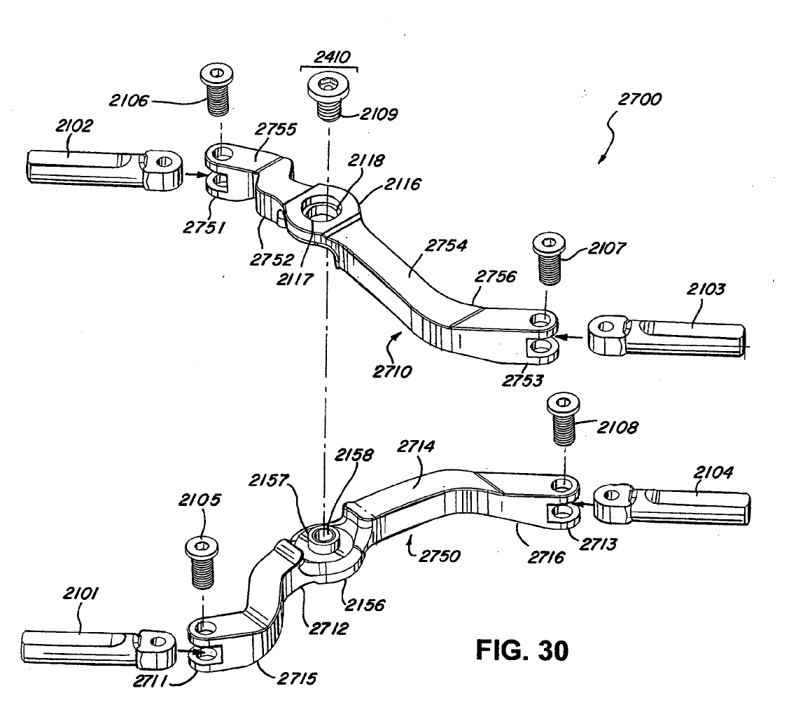

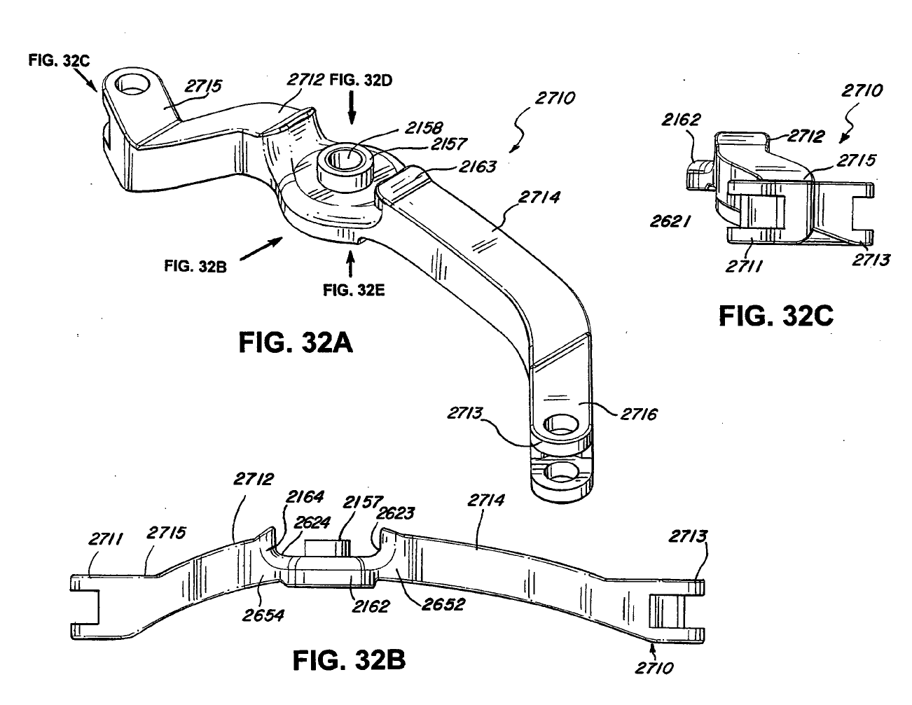

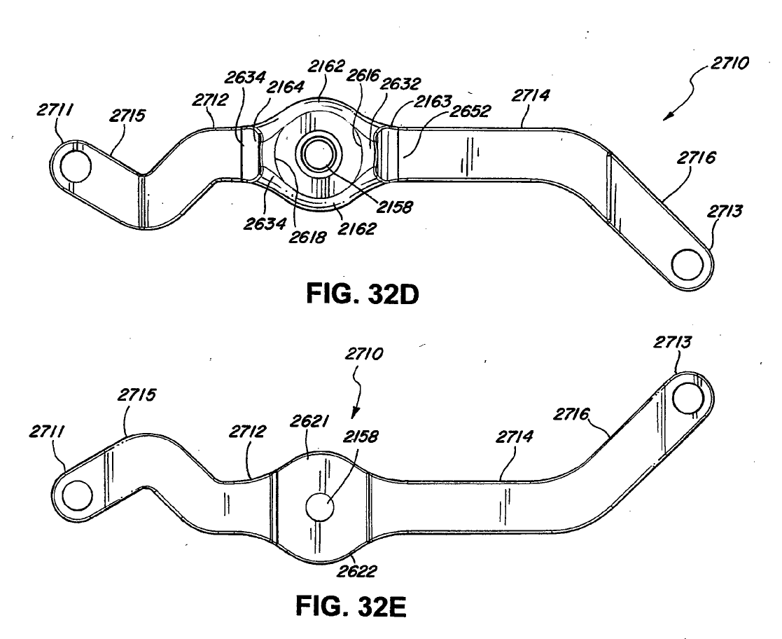

The aforementioned adjustment procedures and ALAD configurations may be applied to each of the ALADs 431, 432, 433, and 434. Advantageously, the RXCC 400 may have a dynamic range of arm length configurations for fitting patients with various spinal bone structures. FIGS. 4G-41 may help illustrate the benefit of the dynamic arm length configurations of the RXCC 400. For example, as shown in FIG. 4G, the RXCC 400 may have a symmetric-Y configuration 486 according to an embodiment of the present invention. With the symmetric-Y configuration 486, the RXCC 400 may be fitted to a patient with spinal bone structure that is symmetric along the Y-axis but asymmetric along the X-axis. More specifically, the first ALAD 431 may have the same arm length configuration 450 as the second ALAD 432 and the third ALAD 433 may have the same arm length configuration 470 as the fourth ALAD 434, while the first ALAD 431 may have a different arm length configuration as the third ALAD 433.

For another example, as shown in FIG. 4H, the RXCC 400 may have a symmetric-X configuration 487 according to an embodiment of the present invention. With the symmetric-X configuration 487, the RXCC 400 may be fitted to a patient with spinal bone structure that is symmetric along the X-axis but asymmetric along the Y-axis. More specifically, the first ALAD 431 may have the same arm length configuration 450 as the third ALAD 433 and the second ALAD 432 may have the same arm length configuration 470 as the fourth ALAD 434, while the first ALAD 431 may have a different arm length configuration as the second ALAD 432.

For yet another example, as shown in FIG. 4I, the RXCC 400 may have a fully asymmetric configuration 488 according to an embodiment of the present invention. With the fully asymmetric configuration 488, the RXCC 400 may be fitted to a patient with spinal bone structure that is asymmetric along the Y-axis and along the X-axis. More specifically, the first ALAD 431 may have a different arm length configuration from the second ALAD 432, which may have a different arm length configuration from the fourth ALAI) 434.

It is understood that the X-axis and the Y-axis are relative terms and they should not be construed to represent any absolute orientation. For example, the Y-axis may be parallel to an approximate orientation of a patient's spine column. For another example, the X-axis may be parallel to the approximate orientation of the patient's spine column.

The discussion now turns to the structural and functional features of the fulcrum member 130. Generally, the fulcrum member 130 may be coupled to the pivot segments 114 and 124. As such, the fulcrum member 130 may perform as a pivot device for facilitating the pivotal movement between the first and second elongated members 110 (or 410) and 120 (or 420) as shown previously.

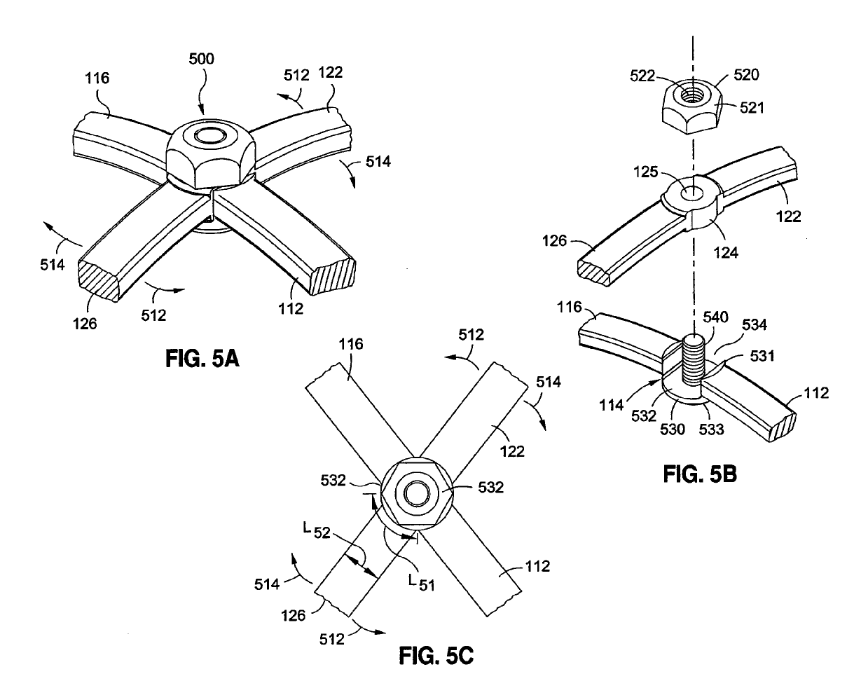

FIGS. 5A-5C show a perspective view, an exploded view, and a top view of a fulcrum member 500, which may be used to realize the fulcrum member 130 according to an embodiment of the present invention. Generally, the fulcrum member 500 may include a cover member 520, a base member 530, and a pivot pole member 540. The cover member 520 may have a top section 522 and an internal threaded section 521 formed along the inner surface cover member 520. The base member 530 may have a bottom section 533, a side wall 531 formed along the edge of the bottom section 533. Moreover, the base member 530 may be formed along the pivot segment 114 of the first elongated member 110, such that the side wall 531 may be attached, coupled, or connected to the first and second ends 112 and 116 of the first elongated member 110. Advantageously, the fulcrum member 500 may be partially integrated with the first elongated member 110 so that the number of assembly components, as well as the number of assembling steps, may be substantially reduced in forming the Real-X cross connector.

As shown in FIG. 5B, the side wall 531 may define a cylindrical space between the top section 521 and the bottom section 533, such that the pivot pin member 540 may be located along a central axis of the cylindrical space. Moreover, the side wall 531 may form a first receiving port 532 and a second receiving port 534 directly opposite to the first receiving port 532. Consequentially, the pivot segment 124 of the second elongated member 120 may be received within the cylindrical space and in between the first and second receiving ports 532 and 534.

As the pivot segment 124 of the second elongated member 120 descends into the receiving ports 532 and 534 of the base member 530, the pivot pin member 540 may penetrate a pivot hole 125 of the second elongated member 120, such that the pivot segment 114 of the first elongated member 110 may engage the pivot segment 124 of the second elongated member 120. When the pivot segment 124 is positioned substantially inside the cylindrical space, the cover member 520 may close the top space of the base member 530 by having the internal threaded section 522 to engage an external threaded section of the pivot pin member 540. Accordingly, the fulcrum member 500 may be formed, such that the second elongated member 120 and the first elongated member 110 may have the relative pivotal movement about the fulcrum member 500.

As shown in FIG. 5C, the second elongated member 120 may have a clockwise angular movement 514 and a counterclockwise angular movement 512 about the first and second openings 532 and 534. Generally, the first and second openings 532 and 534 may each have a width L₅₁ which may be wider than a width L₅₂ of the second elongated member 120. Accordingly, the range of clockwise and/or counterclockwise angular movements 512 and 514 of the second elongated member 120 may be controlled by a difference between the width L₅₁ and L₅₂.

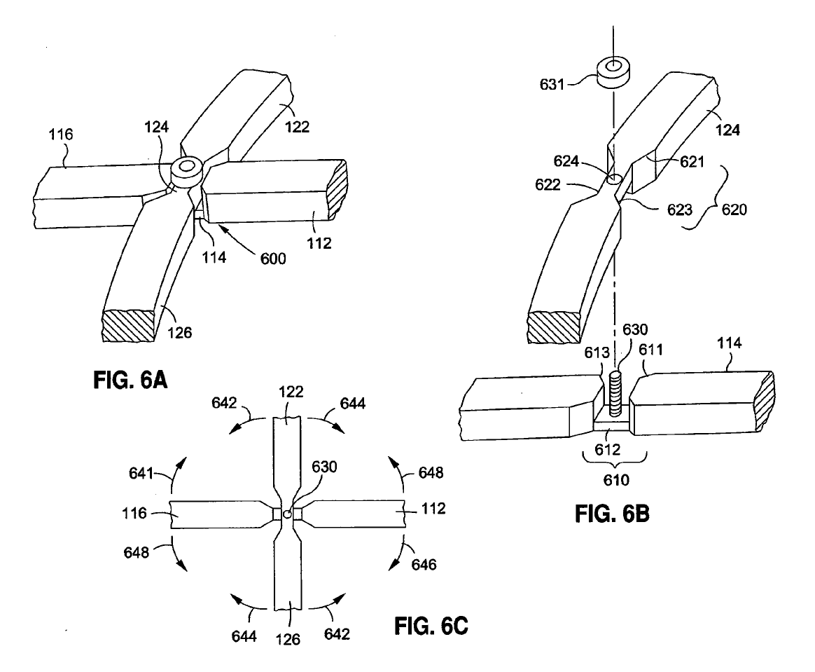

FIGS. 6A-6C show a perspective view, an exploded view, and a top view of an alternative fulcrum member 600, which may be used to realized the functions of the fulcrum member 130 according to an alternative embodiment of the present invention. Generally, the alternative fulcrum member 600 may include a first (bottom) joint member 610, a second (top) joint member 620, a pivot pin member 630 and a pivot cap member 631. As shown in FIGS. 6A and 6B, the first joint member 610 may be formed as part of the pivot segment 114, and the second joint member 620 may be formed as part of the pivot segment 124.

Accordingly, the first joint member 610 may be coupled to the first and second ends 112 and 116 of the first elongated member, and the second joint member 620 may be coupled to the first and second ends 122 and 126 of the second elongated member. Advantageously, the alternative fulcrum member 600 may be fully integrated with the first and second elongated members 110 and 120 so that the number of assembly components, as well as the number of assembling steps, may be substantially reduced.

More specifically, the first joint member 610 may have first and second buffer regions 611 and 613 and a middle bar 612, which may connect the first and second buffer regions 611 and 613. Similarly, the second member 620 may have first and second buffer regions 621 and 623 and a middle bar 622, which may connect the first and second buffer regions 621. In order to facilitate the proper coupling between the first and second joint members 610 and 620, the pivot pin member 630 may be formed on the middle bar 612, and a pivot hole 624 may be extended through the middle bar 622. Alternatively, the pivot pin member 630 may be formed on the middle bar 622, and a pivot hole (not shown) may be defined and extended through the middle bar 612 according to another embodiment of the present invention.

The second joint member 620 may engage the first joint member 610 by allowing the pivot hole 624 to slide down the pivot pin member 630. Because both the middle bars 612 and 622 may have a combined thickness that may be less than or equal to the thickness of the first elongated member 610 or the second elongated member 620, the middle bars 612 and 622 may be free from contacting each other. Additionally, an optional spacer (not shown) may be inserted between the middle bars 612 and 622 to provide additional stability between the first and second joint members 610 and 620. After the first and second joint members 610 and 620 are properly coupled, the pivot cap 631 may be secured to the pivot pin 630 for locking the first and second joint members 610 and 620 together.

As shown in FIG. 6C, the first and second ends 112 and 116 of the first elongated member 610 may have clockwise and counterclockwise angular movements 646 and 648 about the pivot pin member 630. Similarly, the first and second ends 122 and 126 of the second elongated member 620 may have clockwise and counterclockwise angular movements 644 and 642 about the pivot pin member 630. Because the first and second buffer regions 611, 621, 613, and 623 may be slightly sloped, the impact between the first and second elongated members 610 and 620 may be substantially minimized.

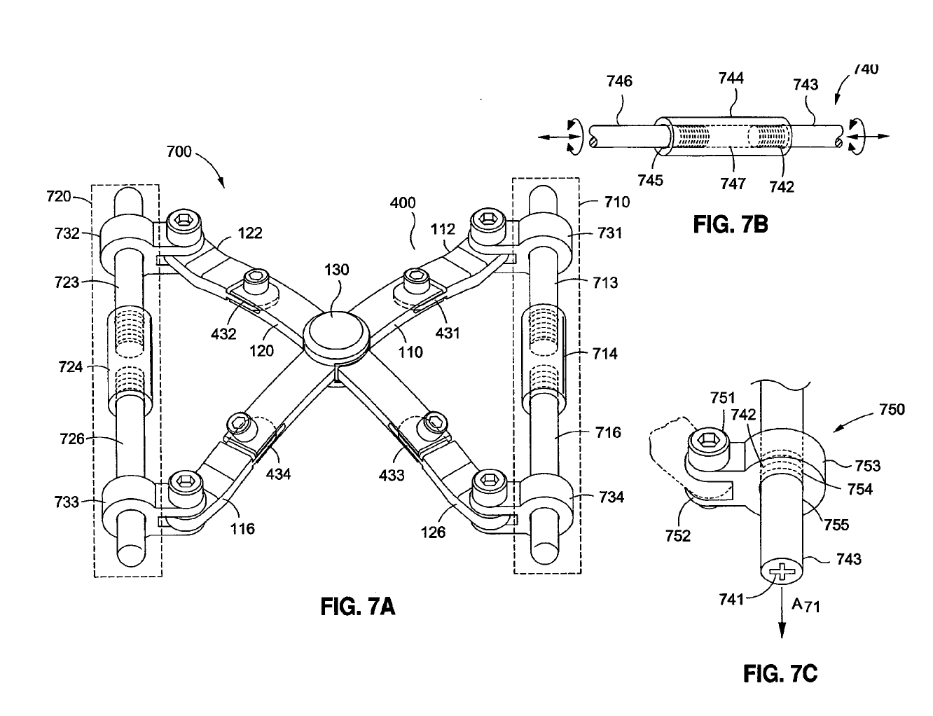

FIGS. 7A-7C show various views of a Real-X cross connector (RXCC) 700 with first and second adjustable rod assemblies (ARAs) 710 and 720 as the connecting devices according to an embodiment of the present invention. Generally, the RXCC 700 may incorporate several structural and functional features of the RXCC 400. For example, the RXCC 700 may incorporate the X-shape protection bridge and the benefits thereof. For another example, the RXCC 700 may incorporate the arm length adjustable devices (ALADs) 431, 432, 433, and 433, and the benefits thereof. Like the RXCC 400, the RXCC 700 may have a dynamic range of arm length configurations for patients with various spinal bone structures.

Despite these similarities, the RXCC 700 may be different from the RXCC 400 in at least one aspect. For example, the RXCC 700 adopted two ARAs 710 and 720 as the connecting devices according to an embodiment of the present invention. From a design standpoint, the ARAs 710 and 720 may provide an integrated solution for conventional cross connectors.

Mainly, the ARAs 710 and 720 may incorporate the structural and functional features of the pair of stabilizing rods 162 and 164 as shown in FIG. 1E as well as the structural and functional features of the several connecting devices discussed so far. As such, the RXCC 700 may be pre-assembled and pre-adjusted according to a surgeon's assessment of a patient's spinal bone structure before the actual spinal fixation surgery is being performed. Advantageously, the ARAs 710 and 720 may improve conventional spinal fixation surgery by reducing the number of surgical steps, the time spent on performing the surgery, and the surgical risk associates with the lengthy surgical procedures.

As shown in FIG. 7A, the first ARA 710 may include first and second articulated ring members 731 and 734, first and second rod segments 713 and 716, and a rod adjustment device 714. Particularly, the first articulated ring member 731 may engage the first rod segment 713, the second articulated ring member 734 may engage the second rod segment 716, and the rod adjustment device 714 may be engaged to both the first and second rod segments 713 and 716. Moreover, the first articulated ring member 731 may be coupled to the first end 112 of the first elongated member 110, and the second articulated ring member 734 may be coupled to the second end 126 of the second elongated member 120.

Similar to the first ARA 710, the second ARA 720 may include first and second articulated ring members 732 and 733, first and second rod segments 723 and 726, and a rod adjustment device 724. Particularly, the first articulated ring member 732 may engage the first rod segment 723, the second articulated ring member 733 may engage the second rod segment 726, and the rod adjustment device 724 may be engaged to both the first and second rod segments 723 and 726. Moreover, the first articulated ring member 732 may be coupled to the first end 122 of the first elongated member 120, and the second articulated ring member 733 may be coupled to the second end 116 of the second elongated member 110.

According to an embodiment, the functions of the rod adjustment devices 714 and 724 may be realized by a rod adjustment assembly 740 as shown in FIG. 7B. Generally, the rod adjustment assembly 740 may include a sleeve member 744, a first insertion member 743, and a second insertion member 746. Particularly, the first insertion member 743 may be coupled to the first rod segment 713 or the first rod segment 723, and the second insertion member 746 may be coupled to the second rod segment 716 or the second rod segment 726.

More particularly, the first and second insertion member 743 and 746 may have external threaded surfaces 742 and 745 respectively, and the sleeve member 744 may have an internal threaded surface 747. When the external threaded surfaces 742 and 745 engage the internal threaded surface 747, the first and second insertion members 743 and 746 may be screwed into or out of the sleeve member 744. Accordingly, the rod adjustment assembly 740 may have an adjustable length depending on the relative positions of the first and second rod segments 743 and 746 with respect to the sleeve member 744.

In one embodiment, the function of the articulated ring members 731, 732, 733, and 734 may be realized by an articulated ring assembly 750 as shown in FIG. 7C. Generally, the articulated ring assembly 750 may have a locking screw 751, a joint member 752, and a ring member 753. Particularly, the joint member 752 may cooperate with the locking screw 751 for engaging and securing one of the first or second end 112, 122, 116, or 126. Depending on the design goal, the joint member 752 may be permanently or temporarily coupled to the ring member 753.

The ring member 753 may have a receiving port 755 for receiving a rod segment 743, which may be one of the first rod segment 713 of the first ARA 710, the second rod segment 716 of the first ARA 710, the first rod segment 723 of the second ARA 720, or the second rod segment 726 of the second ARA 720. Moreover, the ring member 753 may have one or more locking mechanism for preventing the rod segment 743 from sliding pass the receiving port 755 while allowing the rod segment 743 to have a free rotational movement about its central axis A₇₁.

To implement the locking mechanism, the ring member 753 may include one or more protrusion ring(s) 754 disposed along the inner surface of the receiving port 755 according to an embodiment of the present invention. As shown in FIG. 7C, the rod segment 741 may have one or more corresponding intrusion ring(s) 741 for engaging the one or more protrusion ring(s) 754 of the ring member 753. Advantageously, the rod segment 743 may be rotated about the central axis A₇₁ while being secured by the ring member 753.

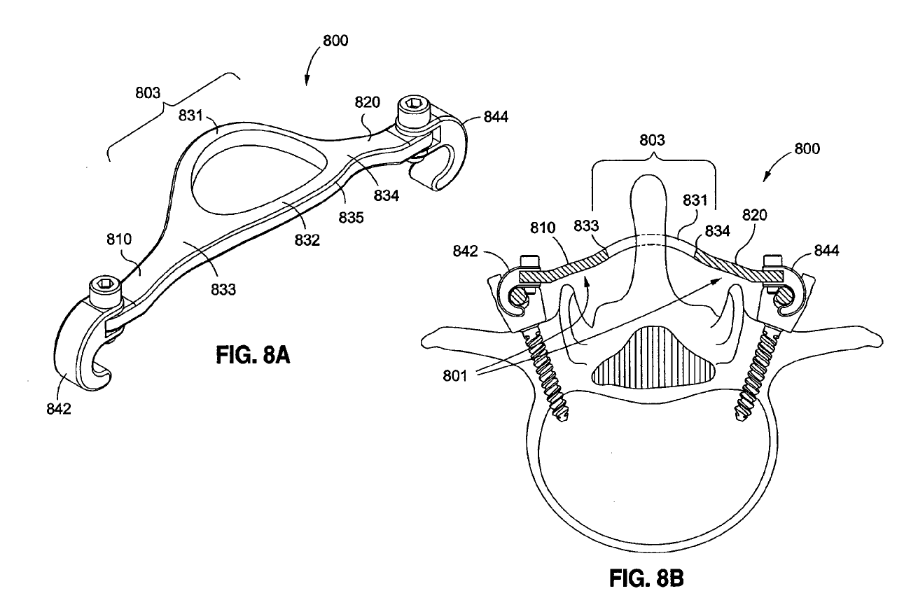

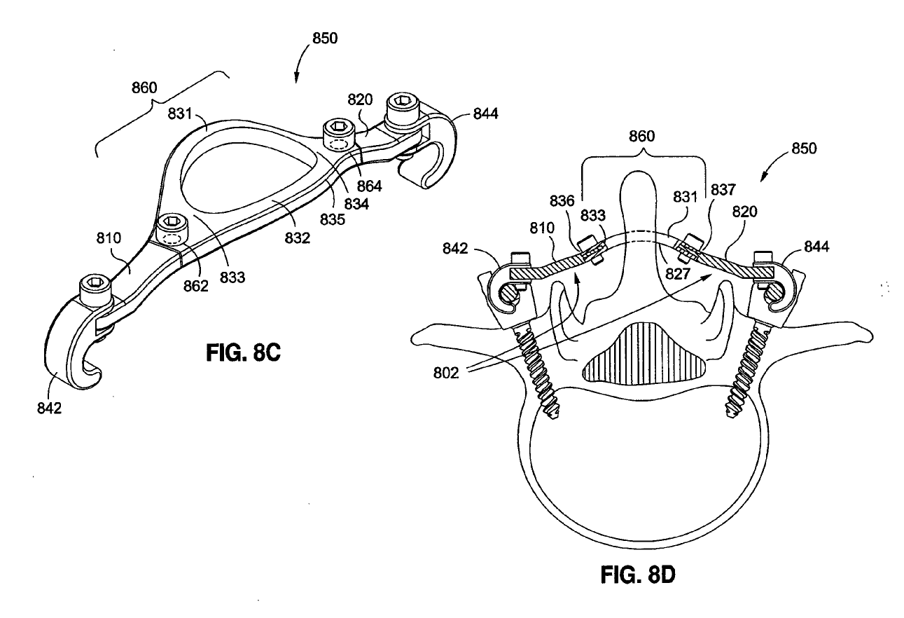

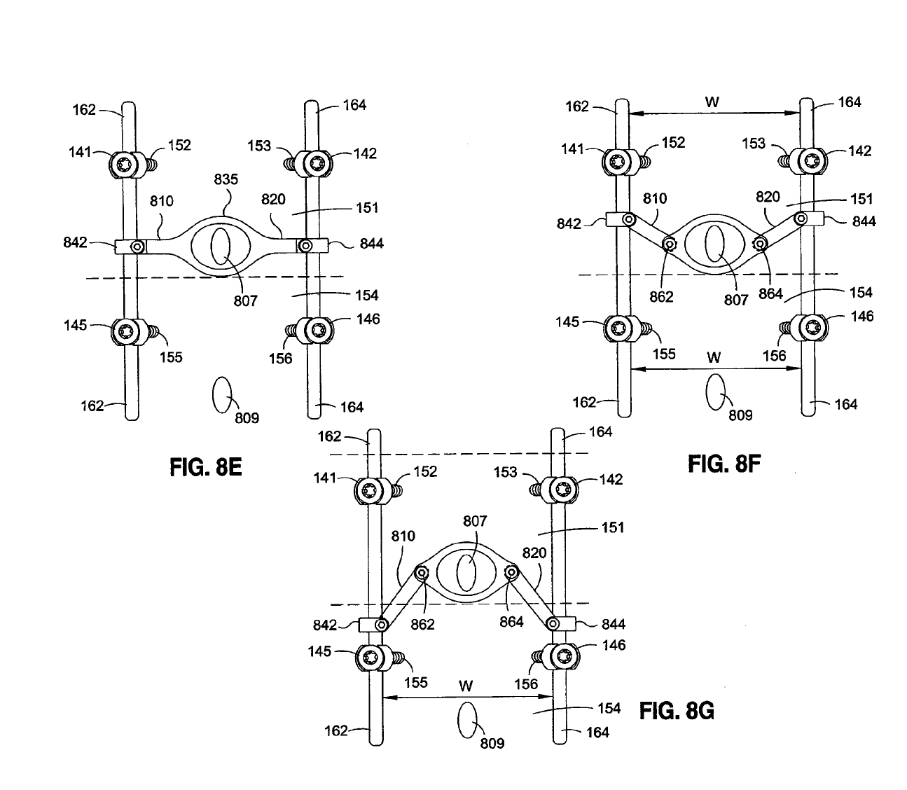

The discussion now turns to a Real-O cross connector (ROCC), which may be used as an alternative device of the Real-X cross connector as discussed previously. FIGS. 8A-8B show a perspective view and a cross sectional side view of a ROCC 800 according to an embodiment of the present invention. Generally, the ROCC 800 may include a center member 803, a first arm 810 and a second arm 820, and first and second anchoring devices 842 and 844. Particularly, the first and second anchoring devices 842 and 844 may be coupled to the first and second arms 810 and 820 respectively. The first and second anchoring devices 842 and 844 may be used for anchoring the ROCC 800 to two stabilizing rods, which may be anchored to several spinal bone segments by several pedicle screws. Accordingly, the structural and functional features of the first and second anchoring devices 842 and 844 may be realized by the anchoring device 240 of FIG. 2B.

In one embodiment, the first and second arm 810 and 820 may be connected to the center member 803 to form an arch bridge 801 as shown in FIG. 8B. The center member 803 may include first and second ends 833 and 834, and first and second bracket 831 and 832, which may join each other at the first and second ends 833 and 834. Together, the first and second brackets 831 and 832 may form a protection ring 835 at the center of the ROCC 800.

The arch bridge 801 may define a space underneath the center member 803, and the protection ring 835 may create an opening at the center of the ROCC 800. Hence, the ROCC 800 may be place direct above a spinal bone segment and may avoid contacting the spinal bone segment's superior articular process, Mamillary process, accessory process, and inferior articular process. Furthermore, the protection ring 835 may help protect and preserve the spinous process by laterally surrounding a base of the spinous process, such that the spinous process of the spinal bone segment may protrude from the protection ring 835. Advantageously, the ROCC 800 may be placed directly across the spinal bone segment without removing the spinous process thereof, and thus, the ROCC 800 may also help prevent symptoms of pseudoarthritis.

Referring to FIG. 8E, the ROCC 800 may be anchored to and positioned in between the first and second stabilizing rods 162 and 164 according to an embodiment of the present invention. Generally, the first stabilizing rod 162 may be anchored to the left pedicles 152 and 155 via the pedicle screws 141 and 145, while the second stabilizing rod 164 may be anchored to the right pedicles 153 and 156 via the pedicle screws 142 and 146. As such, the first and second stabilizing rods 162 and 164 may provide a vertical stabilization for the spinal bone segments 151 and 154.

In order to provide a horizontal stabilization, the ROCC 800 may be anchored to the first stabilizing rod 162 by using the first anchoring device 842 and to the second stabilizing rod 164 by using the second anchoring device 844. Because of the opening defined by the protection ring 835 and the space underneath the arched bridge 801, the ROCC 800 may be conveniently placed above and across the spinal bone segment 151 without removing the spinous process 807 thereof. Advantageously, the ROCC 800 may improve the conventional spinal fixation surgery by making it safer and less intrusive to the patient's body. The above procedure may be repeated for other spinal bone segments. For example, another ROCC 800 may be placed above and across the spinal bone segment 154, such that the protection ring 835 may be placed around the base section of the spinous process 809.

FIGS. 8C-8D show a perspective view and a cross-sectional of an alternative ROCC 850 according to another embodiment of the present invention. Generally, the ROCC 850 may share several structural and functional features with the ROCC 800. For example, the ROCC 850 may have the first and second arms 810 and 820, the first and second anchoring devices 842 and 844, and a center member 860, which may be connected between the first and second arms 810 and 820. For another example, the center member 860 of the ROCC 850 may include the first and second brackets 831 and 832, which may be joined at the first and second ends 833 and 834 respectively to form the protection ring 835. Moreover, the ROCC 850 may form an arched bridge 802, which may have similar structure and provide similar functionalities as the arched bridge 801.

Despite these similarities, the ROCC 850 may be different from the ROCC 800 in at least one aspect. For example, the center member 860 of the ROCC 850 may include a first joint member 862 for engaging the first arm 810 and a second joint member 864 for engaging the second arm 820. Generally, the first and second joint member 862 and 864 may function as two pivoting devices for the protection ring 835.

More specifically, the first and second joint member 862 and 864 may include certain joint mechanism to allow each of the first and second arms 810 and 820 to have a range of angular movement about the first and second ends 833 and 834 so that the ROCC 850 may be adjusted to adapt to various spinal bone structures. Meanwhile, the first and second joint member 862 and 864 may include certain locking mechanism to lock each of the first and second arms 810 and 820 once the ROCC 850 is properly adjusted. In one embodiment, for example, the functional features of the joint members 862 and 863 may be implemented by the joint member 242 as shown and discussed in FIG. 2B.

Referring to FIGS. 8F-8G, the ROCC 850 may be anchored to and positioned in between the first and second stabilizing rods 162 and 164 according to an embodiment of the present invention. Generally, the first stabilizing rod 162 may be anchored to the left pedicles 152 and 155 via the pedicle screws 141 and 145, while the second stabilizing rod 164 may be anchored to the right pedicles 153 and 156 via the pedicle screws 142 and 146. As such, the first and second stabilizing rods 162 and 164 may provide the vertical stabilization for the spinal bone segments 151 and 154, and the ROCC 850 may provide the horizontal stabilization for the first and second stabilizing rods 162 and 164.

In addition to the advantages of the ROCC 800, the ROCC 850 may include other advantages. For example, the joint members 862 and 864 may provide the ROCC 850 with more adjustability in terms of selecting the pair of anchoring points. As shown in FIG. 8F, each of the spinal bone segments 151 and 154 may have a bone width W, which may be shorter than the combined length of the first and second arms 810 and 820. Because the joint members 862 and 864 allow the first and second arms 810 and 820 to fold up or down from the center member 860, the anchoring devices 842 and 844 may established various anchor points along the first and second stabilizing rods 162 and 164.

In order to adapt to the narrow spinal bone segments 151 and 154, the first and second arms 810 and 820 may be folded upward to reach a pair of higher anchored points, so as to reduce the distance between the protection ring 835 and the first and second stabilizing rods 162 and 164. This adjustment process may be repeated for adapting the ROCC 850 to spinal bone segments with a range of spinal bone widths. Advantageously, the ROCC 850 may be installed to patients with spinal bone segments of various widths.

Furthermore, the adjustability provided by the first and second joint members 862 and 864 may allow the ROCC 850 to adapt to asymmetric spinal bone segments. As shown in FIG. 8G, the spinous process 807 of the spinal bone segment 151 may be closer to the left pedicle 152 than to the right pedicle 153. In order to adapt to the asymmetry of the spinal bone segment 152, the first arm 810 may be folded with a larger downward angle than the second arm 820. Accordingly, the distance between the protection ring and the first stabilizing rod 162 may be less than the distance between the protection ring and the second stabilizing rod 164. This adjustment process may be repeated for adapting the ROCC 850 to spinal bone segments with various degrees of asymmetry. Advantageously, the ROCC 850 may be applied to fit patients with asymmetric spinal bone segments.

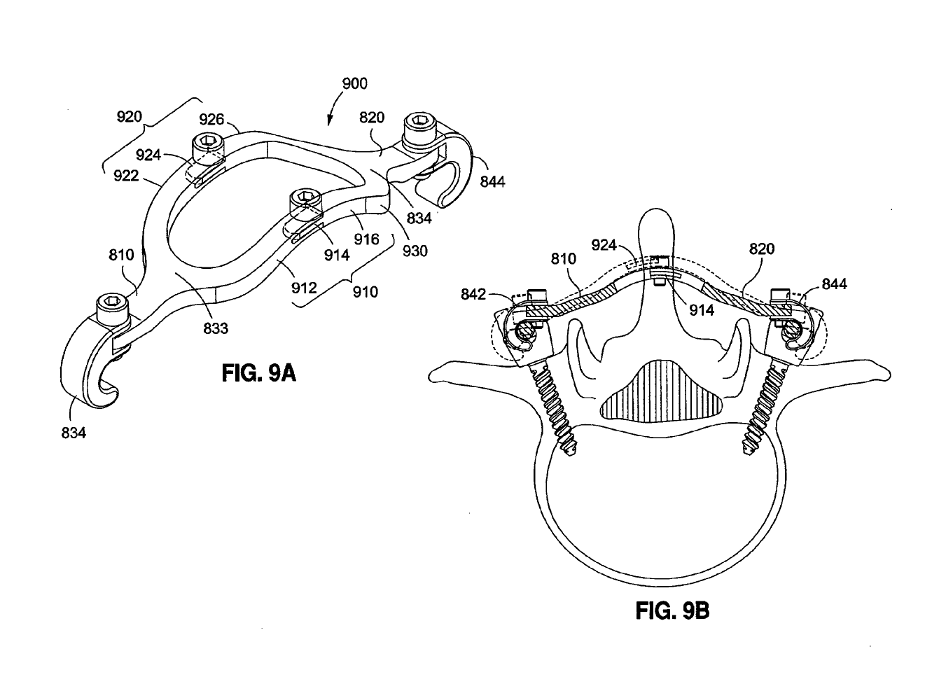

FIGS. 9A-9B show various views of a Real-O cross connector (ROCC) 900 with an adjustable ring according to an embodiment of the present invention. Generally, the ROCC 900 may incorporate the structural and functional features of the ROCC 800 and/or the ROCC 850. Additionally, the ROCC 900 may include an adjustable center member 930 in replacing the center member 803 and/or 860. The adjustable center member 930 may include a first adjustable bracket 910 and a second adjustable bracket 920. More particularly, the first and second adjustable brackets 910 and 920 may have first segments 912 and 922, second segments 916 and 926, and length adjustable devices 914 and 924.

The length adjustable device 914 may engage the first and second segments 912 and 916 of the first adjustable bracket 910, and the length adjustable device 914 may change the relative position between the first and second segments 912 and 916. Accordingly, the length adjustable device 914 may change the length of the first adjustable bracket 910. Similarly, the length adjustable device 924 may engage the first and second segments 922 and 926 of the first adjustable bracket 920, and the length adjustable device 924 may change the relative position between the first and second segments 922 and 926. Accordingly, the length adjustable device 924 may change the length of the first adjustable bracket 920.

The functional features of the length adjustable devices 914 and 924 may be realized by any compatible mechanical components. In one embodiment, for example, the length adjustable devices 914 and 924 may each be implemented by the arm length adjustable device 440 as described and discussed in FIGS. 4B-4F.

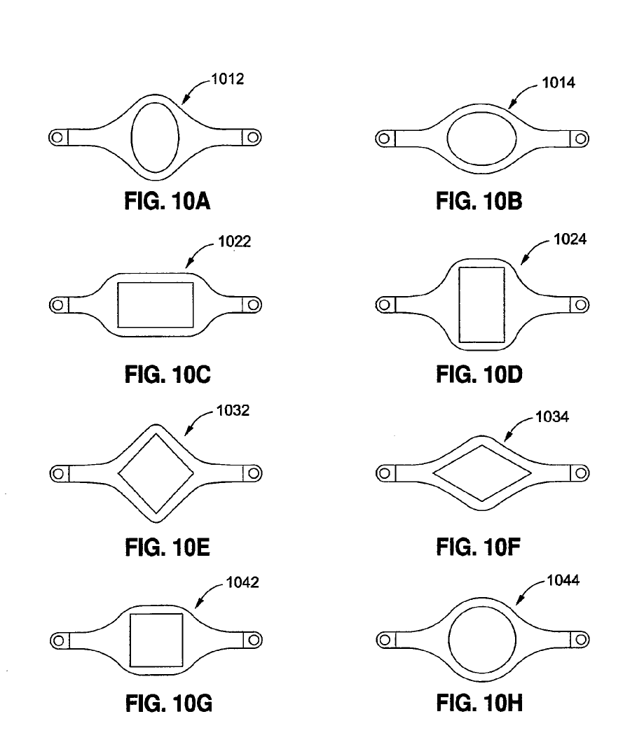

The discussion now turns to the various shapes of the protection rings of the Real-O cross connectors according to various embodiments of the present invention. As shown in FIG. 10A, the protection ring 1012 may, for example, have a shape of a vertical oval. As shown in FIG. 10B, the protection ring 1014 may, for example, have a shape of a horizontal vertical oval. As shown in FIG. 10C, the protection ring 1022 may, for example, have a shape of a horizontal rectangle. As shown in FIG. 10D, the protection ring 1024 may, for example, have a shape of a vertical rectangle. As shown in FIG. 10E, the protection ring 1032 may, for example, have a shape of a vertical rhombus. As shown in FIG. 10F, the protection ring 1034 may, for example, have a shape of a horizontal rhombus. As shown in FIG. 10G, the protection ring 1042 may, for example, have a shape of a square. As shown in FIG. 10H, the protection ring 1044 may, for example, have a shape of a circle. The aforementioned shapes of the protection rings are only for illustrative purpose since the protection ring may have other shapes that may be adaptive to various contour of the base section of the spinous process.

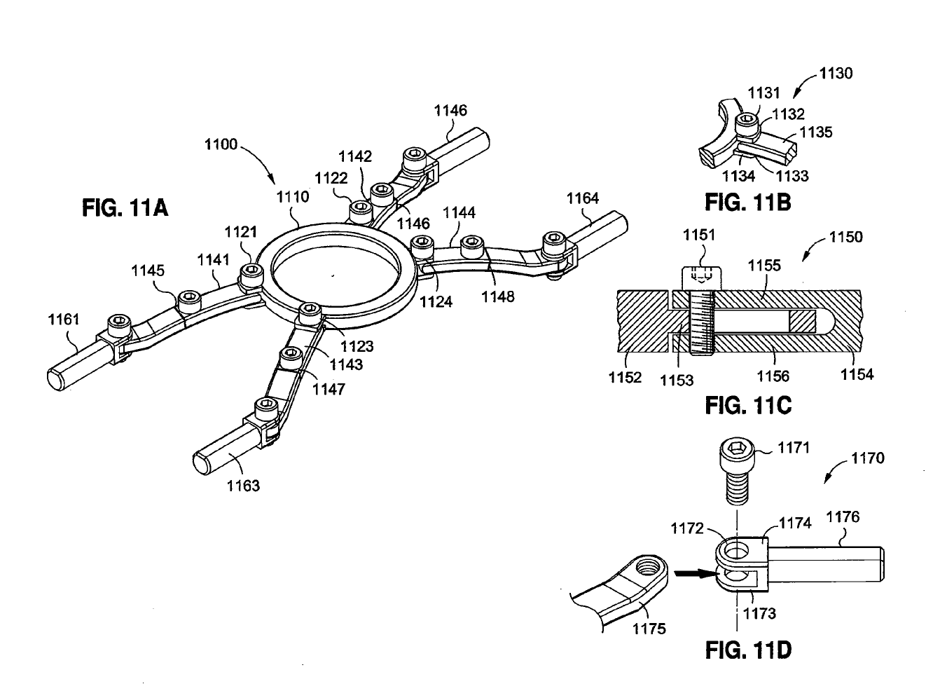

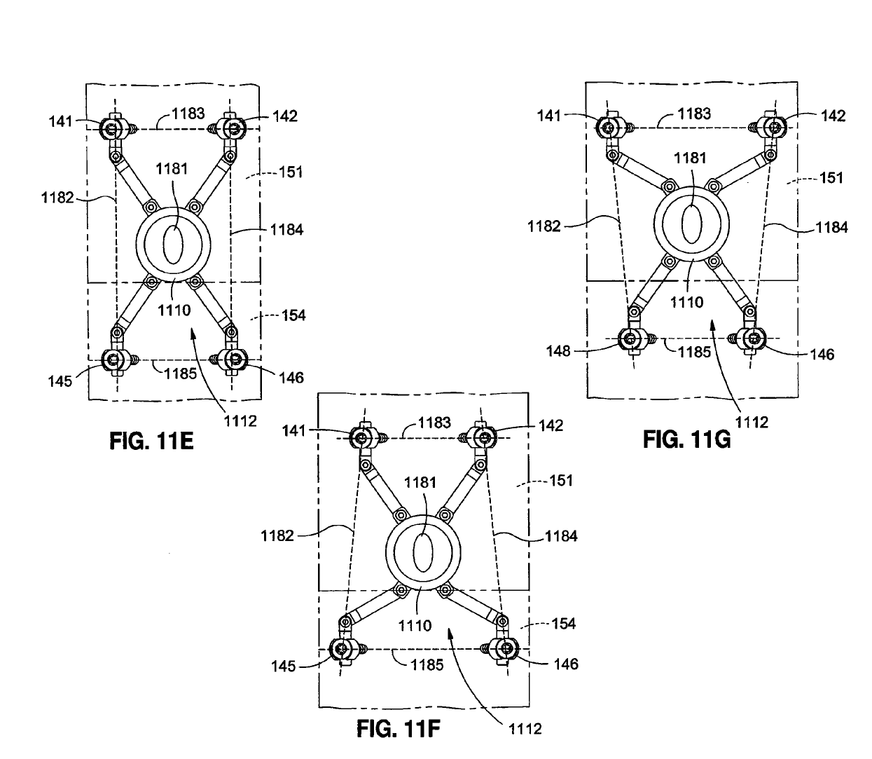

The discussion now turns to a Real-XO cross connector (RXOCC), which may be used as an alternative device of the Real-X cross connector (RXCC) and the Real-O cross connector (ROCC). FIGS. 11A-11D show various views of an RXOCC 1100 according to an alternative embodiment of the present invention. Generally, the RXOCC 1100 may incorporate several structural and functional features of the Real-X cross connectors (RXCC) and the Real-O cross connectors (ROCC) as discussed previously. For example, the RXOCC 1100 may include a protection ring 1110, four joint members 1121, 1122, 1123, and 1124, four elongated members 1141, 1142, 1143, and 1144, four arm length adjustable devices (ALADs) 1145, 1146, 1147, and 1148, and four connecting devices 1161, 1162, 1163, and 1164.

In one embodiment, the joint members 1121, 1122, 1123, and 1124 may secure the elongated members 1141, 1142, 1143, and 1144 to the protection ring 1110. In another embodiment, the ALADs 1145, 1146, 1147, and 1148 may be adjustable so that the elongated members 1141, 1142, 1143, and 1144 may each have an adjustable length. In yet another embodiment, the connecting devices 1161, 1162, 1163, and 1164 may connect the RXOCC to one or more spinal bone segments via several pedicle screws and/or a pair of elongated stabilizers. Although the connecting devices 1161, 1162, 1163, and 1164 are implemented by the articulated rod 1170 as shown in FIG. 11A, they may be implemented by other devices, such as the anchoring device 240 as shown in FIG. 2B.

Specifically, the elongated members 1141, 1142, 1143, and 1144 may be distributed along the edge of the protection ring 1110. When the joint members 1121, 1122, 1123, and 1124 are unlocked, the elongated members 1141, 1142, 1143, and 1144 may be free to be angularly displaced about the respective joint members. Alternatively, the elongated members 1141, 1142, 1143, and 1144 may be free to move along the edge of the protection ring 1110 when the respective joint members 1121, 1122, 1123, and 1124 are unlocked. When the joint members 1121, 1122, 1123, and 1124 are locked, the elongated members 1141, 1142, 1143, and 1144 may each be affixed to a particular position in relative to the protection ring 1110.

At the locking mode, the RXOCC 1100 may form a hybrid X-shaped protection bridge, which may arch over a space directly underneath the protection ring 1110 while allowing the space to extend through an opening defined by the protection ring 1110. Advantageously, the hybrid X-shaped protection bridge may inherit the benefits of the Real-X cross connector (RXCC) and the Real-O cross connector (ROCC).

As shown in FIG. 11B, the four joint members 1121, 1122, 1123, and 1124 may each be implemented by a lockable joint 1130 according to an embodiment of the present invention. The lockable joint 1130 may include a locking screw 1131, a first plate 1132, a second plate 1133, and a side body 1134. The side body 1134 may be coupled to the edge of the protection ring 1110, such that the lockable joint 1130 may receive an end member 1135 along an outer circumferential surface (the edge) of the protection ring 1110. As discussed herein, the end member 1135 may be one of the first, second, third, or fourth elongated member 1141, 1142, 1143, or 1144. Moreover, the first and second plates 1132 and 1133 may be separated by a space for receiving the end member 1135, and they may each have an opening for receiving the locking screw 1131.

Before the locking screw 1131 substantially engages the second plate 1133, the end member 1135 may be freely rotated about the locking joint member 1130. Correspondingly, the first, second, third, and fourth elongated members 1141, 1142, 1143, and 1144 may be adjusted to different angular positions with respect to the protection ring 1110. Advantageously, the RXOCC 1100 may be adjustable to form X-shape protection bridges with various angular positions.

In order to lock the lockable joint 1130, the locking screw 1131 may be used for substantially engaging the second plate 1133. The locking screw 1131 may cooperate with the second plate 1133 to produce a pair of compression forces, which may be asserted against the end member 1135. As such, the frictional forces between the end member 1145 and the inner surfaces of the first and second plates 1132 and 1133 may be increased significantly. As a result, the end member 1135 may be locked in a particular position with respect to the lockable joint member 1130. Correspondingly, the first, second, third, and fourth elongated members 1141, 1142, 1143, and 1144 may each be locked at a particular angularly position with respect to the protection ring 1110.

FIG. 11C shows a cross-sectional side view of an ALAD 1150, which may realize the functional features of the first, second, third and fourth ALADs 1145, 1146, 1147, and 1148. In one embodiment, for example, the ALAD 1150 may include the same components as the ALAD 440 (see FIGS. 4B and 4C), and it may thus incorporate the functional features of the ALAD 440. Generally, the ALAD 1150 may include a locking screw 1151 a male member 1152, which may have an insertion member 1153, a female member 1154, which may have first and second plates 1155 and 1156 to define a space for receiving the insertion member 1153.

More specifically, the insertion member 1153 may be slid in and out of the space before the locking screw 1151 substantially engages the second plate 1156. As such, the distance between the male and female member 1152 and 1154 may be adjusted. However, when the locking screw 1151 substantially engages the second plate 1156, the insertion member 1153 may be locked within a particular position within the space defined within the female member 1154. Accordingly, the male and female members 1152 and 1154 may be substantially stabilized and they may thus form an adjusted distance between them.

FIG. 11D shows a cross-sectional side view of an articulated rod 1170, which may realize several functional features of the first, second, third, and fourth connecting devices 1161, 1162, 1163, and 1164 as discussed earlier. In one embodiment of the present invention, for example, the articulated rod 1170 may include the same components as the articulated rod 340 (see FIGS. 3B and 3C), and it may thus incorporate the functional features of the articulated rod 340. Generally, the articulated rod 1170 may include a lockable joint member 1174 and a rod member 1176, which may be connected to the lockable joint member 1174.

The lockable joint member 1174 may be similar to the lockable joint member 1130. As such, the lockable joint member 1174 may be used to secure an end member 1175, which may be one of the first, second, third, or fourth elongated member 1141, 1142, 1143, or 1144. Specifically, the locking joint member 1171 may include first and second plates 1172 and 1173, which may define a space for receiving the end member 1175, and a locking screw 1171 for locking the end member 1175 between the first and second plates 1172 and 1173. The rod member 1176 may share similar functionalities as a conventional stabilizing rod such that the rod member 1176 may be received and secured by a conventional pedicle screw, which may be anchored to a spinal bone segment.

Because the RXOCC 1100 may be fully adjustable before the several locking mechanisms are applied, the X-shape protection bridge 1112 may have several configurations for fitting patients with various spinal bone structures. In FIG. 11E, the spinal bone segments 151 and 154 may have a pair of parallel inter-segment lines and a pair of parallel intra-segment lines. The pair of inter-segment lines may include a first inter-segment line 1182 defined by the pedicle screws 141 and 145, and a second inter-segment line 1184 defined by the pedicle screws 142 and 146. Moreover, the pair of intra-segment lines may include a first intra-segment line 1181 defined by the pedicle screws 141 and 142, and a second intra-segment line 1185 defined by the pedicle screws 145 and 146. As such, the X-shape protection bridge may have a fully symmetrical configuration according to an embodiment of the present invention, and in which the protection ring 1110 may surround a base section of a spinous process 1181 of the spinal bone segment 151.

Referring to FIG. 11F, the spinal bone segments 151 and 154 may have a pair of diverging intra-segment lines 1182 and 1184 and a pair of parallel inter-segment lines 1183 and 1185. As such, the X-shape protection bridge may be adjusted to have a partial symmetrical configuration according to another embodiment of the present invention. Referring to FIG. 11G, the spinal bone segments 151 and 154 may have a pair of diverging intra-segment lines 1182 and 1184 and a pair of diverging inter-segment lines 1183 and 1185. As such, the X-shape protection bridge may be adjusted to have a fully asymmetrical configuration according to yet another embodiment of the present invention.

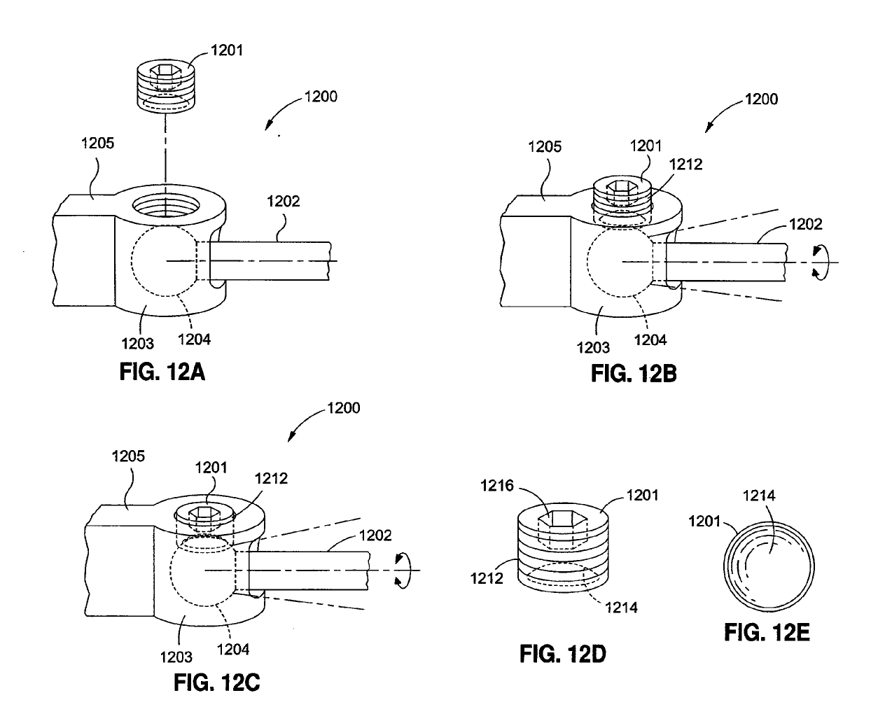

The discussion now turns to an alternative lockable joint member. Although the lockable joint member with the two-plate configuration has been discussed with respect to various embodiments of the present invention, an alternative lockable joint member with a multi-axial joint may be used for realizing several functional features of the lockable joint member. As shown in FIG. 12A, an alternative lockable joint member 1200 may generally include a locking screw 1201, a housing 1205, a socket 1203 located within the housing 1202, a bearing 1204, and a handle member 1202. More specifically, the housing may have a top surface and a side wall, such that a top receiving port may be formed on the top surface and a side receiving port may be formed on the side wall.

As shown in FIG. 12B, the socket 1203 may receive the bearing 1204, and it may have a socket surface for contacting the bearing 1204 and thereby allowing the bearing 1204 to rotate therein. The handle member 1202 may be coupled to the bearing 1204 and it may protrude from the side wall of the housing 1205 via the side receiving port. The handle member 1202 may have a range of multi-axle movement about a center of the bearing 1204 or about the side receiving port. Depending on the other functions of the lockable joint member 1200, the housing 1205 may be coupled to a rod member in one embodiment or a hook member in another embodiment. The handle member 1202 may be coupled to an end of an elongated member (arm), such that the housing 1205 may rotate about the end of the elongated member.

As shown in FIG. 12C, the locking screw 1201 may descend into the top opening of the housing 1205. When the external threaded section 1212 of the locking screw 1201 substantially engages the internal threaded section of the housing 1205, the inner concave surface 1214 may assert a compression force against the bearing 1204. Consequentially, the compression force may cooperate with the surface of the socket 1203 to lock the bearing 1204 at a particular position.

As shown in FIG. 12D, the locking screw 1201 may have a bearing socket 1216 for receiving a driving force. The driving force may cause the external threaded section 1212 of the locking screw 1201 to substantially engage the internal threaded section of the housing 1205. In FIG. 12E, which shows the bottom view of the locking screw 1201, the bottom concave surface 1214 may be used for engaging the bearing 1204 and thus locking the bearing 1204 in a particular position. In one embodiment, the bottom concave surface 1214 may be distributed with compressible rings. In another embodiment, the bottom concave surface 1214 may be distributed with small protrusions. In yet another embodiment, the inner concave surface 1214 may be a rough surface, which may cause a significant amount of friction upon contact.

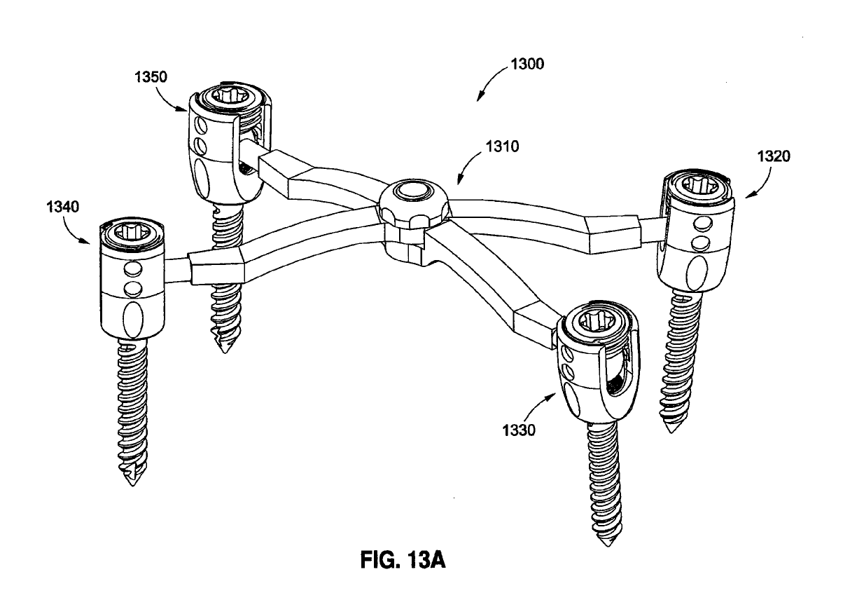

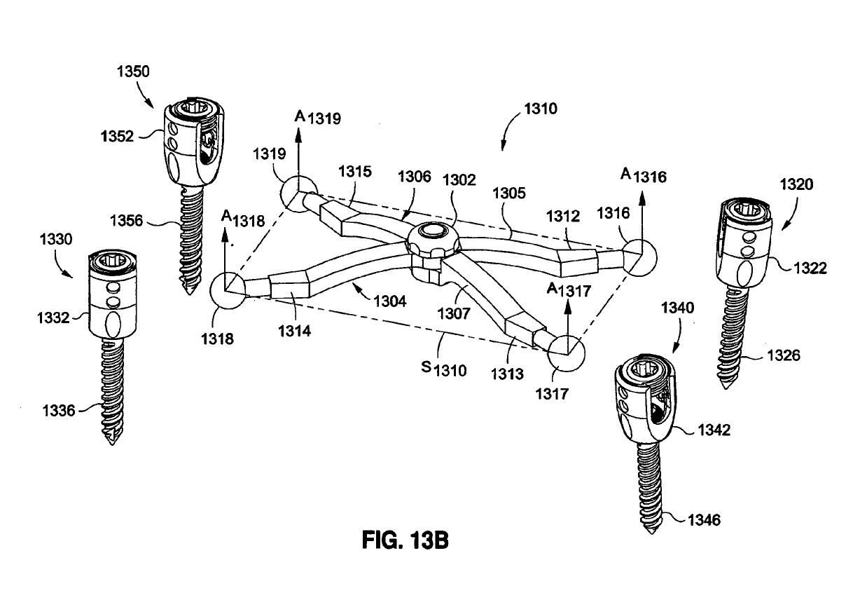

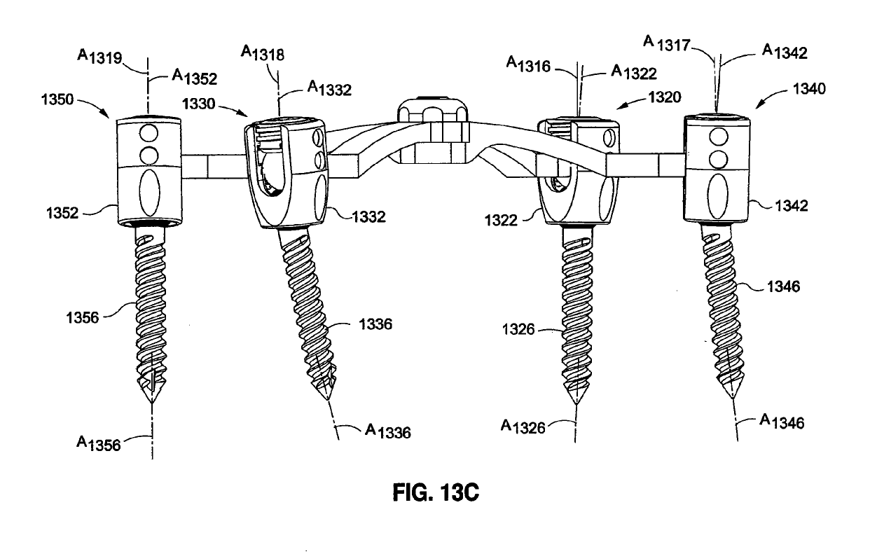

The discussion now turns to a cross connecting pedicle screw system, which may be used for stabilizing and protection one or more fixation levels of spinal bone segments. In FIG. 13A, a perspective view of a Real-X cross connecting pedicle screw (RXCCPS) system 1300 is shown according to an embodiment of the present invention. From a high level standpoint, the RXCCPS system 1300 may incorporate some of the functions of the Real-X cross connector and the pedicle screws. For example, the RXCCPS system 1300 may be anchored to two or more spinal bone segments. For another example, the RXCCPS system 1300 may provide vertical and horizontal fixations to the spinal bone segments.

Generally, the RXCCPS 1300 may include a Real-X cross connector 1310 and four joint receiving (JR) pedicle screws 1320, 1330, 1340, and 1350. The JR pedicle screws 1320, 1330, 1340, and 1350 may be used for anchoring the Real-X cross connector 1310 to two or more spinal bone segments. The Real-X cross connector 1310 may stabilize the relative positions among the four JR pedicle screws 1320, 1330, 1340, and 1350. As a result, the RXCCPS system 1300 may be used for substantially stabilizing two or more spinal bone segments.

FIG. 13B shows a semi-exploded view of the RXCCPS system 1300. Generally, the Real-X cross connector 1310 may include a first elongated member 1304, a second elongated member 1306, and a fulcrum member 1302. The first elongated member 1304 may be a single structure, which may include a first arched segment 1305 connecting to first and second flat ends 1312 and 1314, a first spherical joint 1316 connecting to the first flat end 1312, and a second spherical joint 1318 connecting to the second flat end 1314. Similarly, the second elongated member 1306 may also be a single structure, which may include the second arched segment 1305 connecting to third and fourth flat ends 1313 and 1315, a third spherical joint 1317 connecting to the third flat end 1313, and a fourth spherical joint 1319 connecting to the fourth flat end 1315.

The fulcrum member 1302 may engage and pivot the first and second arched segments 1305 and 1307, such that the first and second elongated members 1304 and 1306 may form an adjustable X-shape bridge. Particularly, the first and second elongated members 1304 and 1306 may have a scissor-like movement, which may be advantageous for adapting to patients with various spinal bone widths. Moreover, the first and second elongated members 1304 and 1306 may each have an adjustable length (see FIGS. 4A-41), which may be advantageous for adapting to patients with asymmetric spinal bone configurations.

The centers of the first, second, third, and fourth spherical joints 1316, 1317, 1318, and 1319 may define a base plane S₁₃₁₀. The adjustable X-shaped bridge may arch over the base plane S₁₃₁₀, which may be occupied by two or more spinal bone segments. As such, the adjustable X-shaped bridge may extend across and protect one or more fixation levels of the spinal bone segments.

Moreover, the first spherical joint 1316 may define a first joint axis A₁₃₁₆, the second spherical joint 1318 may define a second joint axis A₁₃₁₈, the third spherical joint 1317 may define a third joint axis A₁₃₁₇, and the fourth spherical joint 1319 may define a fourth joint axis A1319. The first, second, third, and fourth joint axes A₁₃₁₆, A₁₃₁₈, A₁₃₁₇, and A₁₃₁₉ may be substantially perpendicular to base plane S₁₃₁₀, and they may represent the orientations of the respective first, second, third, and fourth spherical joints 1316, 1318, 1317, and 1319.

The four joint receiving (JR) pedicle screws may include a first JR pedicle screw 1320, a second JR pedicle screw 1330, a third JR pedicle screw 1340, and a fourth JR pedicle screw 1350. The first JR pedicle screw 1320 may have a cradle 1322 for engaging the first spherical joint 1316 and a threaded shaft 1326 for anchoring the cradle 1322 to a first spinal bone segment. The second JR pedicle screw 1330 may have a cradle 1332 for engaging the second spherical joint 1318 and a threaded shaft 1336 for anchoring the cradle 1332 to a second spinal bone segment. The third JR pedicle screw 1340 may have a cradle 1342 for engaging the third spherical joint 1317 and a threaded shaft 1346 for anchoring the cradle 1342 to the second spinal bone segment. The fourth JR pedicle screw 1350 may have a cradle 1352 for engaging the fourth spherical joint 1319 and a threaded shaft 1356 for anchoring the cradle 1352 to the first spinal bone segment.

Generally, the first, second, third, and fourth JR pedicle screws 1320, 1330, 1340, and 1350 may each have a multi-axle movement about the respective first, second, third, and fourth spherical joints 1316, 1318, 1317, and 1319. Particularly, the cradles 1322, 1332, 1342, and 1352 may rotate about the respective first, second, third, and fourth joint axes A₁₃₁₆, A₁₃₁₈, A₁₃₁₇, and A₁₃₁₉. Because the cradles 1322, 1332, 1342, and 1352 may be fully adjustable around the first, second, third, and fourth spherical joints 1316, 1318, 1317, and 1319, the RXCCPS system 1300 may be used under a wide range of pedicle insertion angles.

In FIG. 13C, a side view of the RXCCPS system 1300 is shown according to an embodiment of the present invention. The first JR pedicle screw 1320 may have a cradle axis A₁₃₂₂ defined by the cradle 1322 and a shaft axis A₁₃₂₆ defined by the threaded shaft 1326. The second JR pedicle screw 1330 may have a cradle axis A₁₃₃₂ defined by the cradle 1332 and a shaft axis A₁₃₃₆ defined by the threaded shaft 1336. The third JR pedicle screw 1340 may have a cradle axis A₁₃₄₂ defined by the cradle 1342 and a shaft axis A₁₃₄₆ defined by the threaded shaft 1346. The fourth JR pedicle screw 1350 may have a cradle axis A₁₃₅₂ defined by the cradle 1352 and a shaft axis A₁₃₅₆ defined by the threaded shaft 1356.

The joint axis, the cradle axis and the shaft axis may align with one another when no adjustment is made to a particular spherical joint. However, the shaft axis may deviate from the cradle axis to achieve a first multi-axle movement, and the cradle axis may deviate from the joint axis to achieve a second multi-axle movement. Accordingly, the RXCCPS 1300 may provide two levels of multi-axle movement, and it may thus improve the adjustability and flexibility of conventional pedicle screw and stabilizing rod systems.

For example, regarding the first RJ pedicle screw 1320, the shaft axis A₁₃₂₆ may align with the cradle axis A₁₃₂₂. As such, the threaded shaft 1326 may sustain a minimal first multi-axle movement. However, the cradle axis A₁₃₂₂ may deviate from the first joint axis A₁₃₁₆, such that the cradle 1322 may achieve a limited second multi-axle movement.

For another example, regarding the second RJ pedicle screw 1330, the shaft axis A1336 may deviate from the cradle axis A₁₃₃₂. As such, the threaded shaft 1336 may achieve a limited first multi-axle movement. However, the cradle axis A₁₃₃₂ may align with the second joint axis A₁₃₁₅, such that the cradle 1332 may sustain a minimal second multi-axle movement.

For another example, regarding the third RJ pedicle screw 1340, the shaft axis A₁₃₄₆ may deviate from the cradle axis A₁₃₄₂. As such, the threaded shaft 1346 may achieve a limited first multi-axle movement. Moreover, the cradle axis A₁₃₄₂ may deviate from the third joint axis A₁₃₁₇, such that the cradle 1342 may achieve a limited second multi-axle movement.

For yet another example, regarding the fourth RJ pedicle screw 1350, the shaft axis A₁₃₅₆ may align with the cradle axis A₁₃₅₂. As such, the threaded shaft 1356 may sustain a minimal first multi-axle movement. Moreover, the cradle axis A₁₃₅₂ may align with the fourth joint axis A₁₃₁₉, such that the cradle 1352 may sustain a minimal second multi-axle movement.

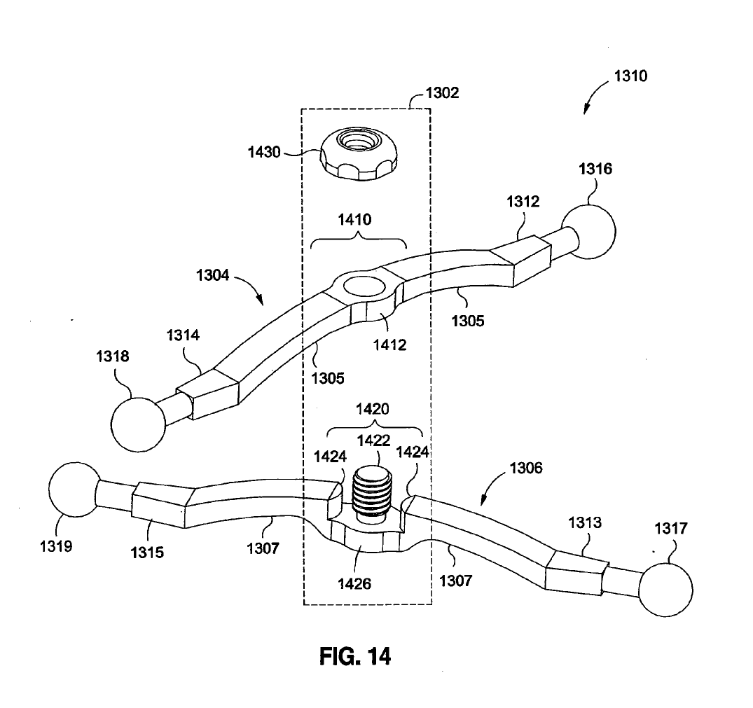

The discussion now turns to the structural and functional features of the Real-X cross connector 1310. FIG. 14 shows an exploded view of the Real-X cross connector 1310 with an integrated fulcrum member 1302. Generally, the first elongated member 1304 may include a first pivot member 1410 positioned within the first arched segment 1305, and the second elongated member 1306 may include a second pivot member 1420 positioned within the second arched segment 1307. The first and second pivot members 1410 and 1420 may pivot each other so as to facilitate a relative movement between the first and second elongated members 1304 and 1306. The first and second pivot members 1410 and 1420 may be implemented with various structures capable of actuating a scissor-like motion between the first and second elongated members 1304 and 1306.

For example, the first pivot member 1410 may include a pivot ring 1412, and the second pivot member 1420 may include a pivot base 1426, a pivot pin 1422 attached on the pivot base 1426, and a pair of pivot alignment bumps 1424. Particularly, the pivot pin 1422 may be used for engaging and pivoting the pivot ring 1412, and the pair of pivot alignment bumps 1412 may contact and guide the pivoting movement of the pivot ring 1412. In order to secure the first elongated member 1304 to the second elongated member 1305, a cap 1430 may be used for engaging the pivot pin 1422.

Moreover, the cap 1430 may be used for substantially restricting the relative movement between the first and second elongated members 1304 and 1305. The cap 1430 may press the pivot ring 1412 against the pivot base 1426 by substantially engaging the pivot pin 1422. This may increase the frictional force between the pivot ring 1422 and the pivot base 1426 and the frictional force between the pivot ring 1422 and the cap 1430. As a result, the increased frictional forces may lock the first and second elongated members 1304 and 1306 at a particular position to form a rigid X-shaped bridge.

Although FIG. 14 shows that the first and second elongated members 1304 and 1306 are two single-piece components, the first and second elongated members 1304 and 1306 may incorporate other components to enhance the functionalities thereof. For example, the first and second arched segments 1305 and 1307 may incorporate one or more arm-length adjustment devices (ALAD), which may be used for adjusting the length and curvature thereof. For another example, each of the first, second, third, and fourth flat ends 1312, 1314, 1313, and 1315 may incorporate a flexible joint, which may be used for adjusting the orientations of the first, second, third, and fourth spherical joints 1316, 1318, 1317, and 1319.

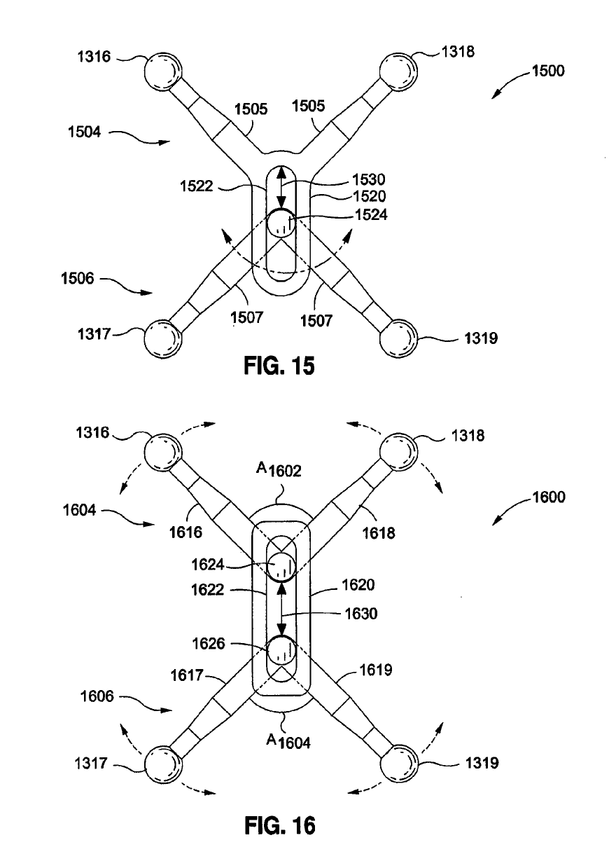

In FIG. 15, a top view of a semi-adjustable length Real-X cross connector 1500 is shown according to an embodiment of the present invention. Generally, the Real-X cross connector 1500 may include a first elongated member 1504, a second elongated member 1506, and a fulcrum member 1520. The first elongated member 1504 may include a first V-shaped arched segment 1505, which may be coupled to the first and second spherical joints 1316 and 1318. The second elongated member 1506 may include a second V-shaped arched segment 1507, which may be coupled to the third and fourth spherical joints 1317 and 1319. Together, the first and second V-shaped arched segments 1505 and 1507 may form the X-shaped bridge.

The first elongated member 1504 may be combined with the fulcrum member 1520, which may include a channel 1522 and a knob 1524. When the knob is relaxed, the peak of the second V-shaped arched segment 1507 may travel along the channel 1522. As such, the knob 1524 may be used for adjusting a peak-to-peak length 1530, which is measured between the peaks of the first and second V-shaped arched segment 1505 and 1507. Moreover, the second V-shaped arched segment 1507 may rotate about the knob 1524. The fulcrum member 1520 may facilitate a relative movement between the first and second elongated members 1504 and 1506, so that they may be adjusted to adapt to patients with various spinal bone configurations. After the proper adjustment is made, the knob 1524 may be tightened to restrict the relative movement between the first and second elongated members 1504 and 1506.

In FIG. 16, a top view of a fully adjustable Real-X cross connector 1600 is shown according to an embodiment of the present invention. Generally, the fully adjustable Real-X cross connector 1600 may include a first elongated member 1604, a second elongated member 1606, and a fulcrum member 1620. The first elongated member 1604 may include a first semi-arched segment 1616 connected to the first spherical joint 1316 and a second semi-arched segment 1618 connecting to the second spherical joint 1318. Similarly, the second elongated member 1606 may include a third semi-arched segment 1617 connecting to the third spherical joint 1316 and a fourth semi-arched segment 1619 connecting to the fourth spherical joint 1319. The fulcrum member 1620 may include a channel 1622, a first knob 1624, and a second knob 1626.

The first knob 1624 may be used for adjusting a first angle A₁₆₀₂ between the first and second semi-arched segments 1616 and 1618. Similarly, the second knob 1626 may be used for adjusting a second angle A₁₆₀₄ between the third and fourth semi-arched segments 1617 and 1619. Together, the first and second knobs 1624 and 1626 may be used for controlling the peak-to-peak distance 1630 between the first and second elongated members 1604 and 1606. Accordingly, the spherical joints 1316, 1318, 1317, and 1319 may be adjusted angularly and longitudinally, so that the fully adjustable Real-X cross connector 1600 may adapt to patients with various spinal bone configurations.

Although FIGS. 13A-13B and FIGS. 14-16 show that the Real-X cross connector is used in the RXCCPS system 1300, the Real-O cross connector and/or the Real-XO cross connector may be used in forming alternative cross connecting pedicle screw systems. For example, the alternative cross connecting pedicle screw systems may include a ring member, which may be used for surrounding and preserving the spinous process of the patient. More specifically, the connecting devices of the Real-O cross connector and/or the Real-XO cross connector may be replaced by the spherical joints 1316, 1318, 1317, and 1319. To that end, the conventional pedicle screws may be replaced by the JR pedicle screws 1320, 1330, 1340, and 1350. Accordingly, the alternative cross connecting pedicle screw systems may incorporate the functional features of the Real-O and Real-XO connectors and the advantages provided by the cross connector spherical joints and the RJ pedicle screws.

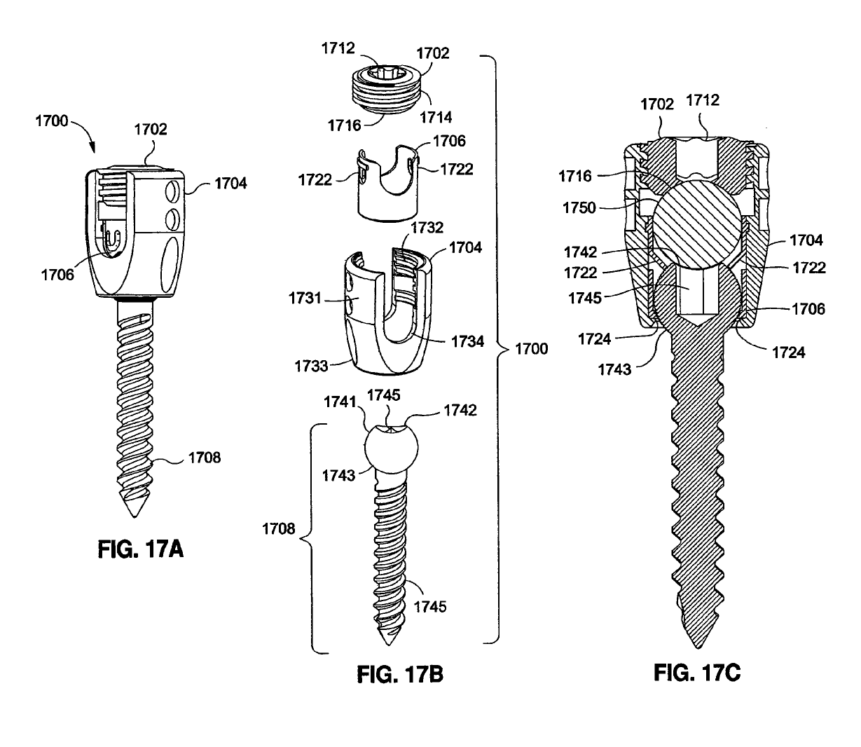

The discussion now turns to structural and functional features of the joint receiving (JR) pedicle screws. FIGS. 17A-17C show various views of the JR pedicle screw 1700 according to an embodiment of the present invention. Generally, the JR pedicle screw 1700 may include a set screw 1702, a cradle 1704, a cylindrical adaptor 1706, and a screw member 1708. The cradle 1704 may include a side wall 1731 and a base 1733. Together, the side wall 1731 and the base 1733 may define a cylindrical space and a cradle axis along the cylindrical space. The cylindrical adaptor 1706 may have a pair of locking members (locking flanges) 1722, and it may be secured within the cylindrical space defined by the cradle 1704.

The side wall 1731 of the cradle 1704 may have an inner threaded surface 1732 for engaging the set screw 1702 and one or more receiving ports 1734 for receiving the spherical joint 1750, which may be one of the four spherical joints 1316, 1318, 1317, and 1319 as shown in FIG. 13B. Particularly, the size of the receiving ports 1734 may limit the second multi-axle movement (See FIG. 13C) between the cradle 1704 and the spherical joint 1750.

The screw member 1708 may include a semi-spherical joint 1741 and a threaded shaft 1745. The semi-spherical joint 1741 may have a first concave surface 1742, a hemispherical surface 1743 formed on the opposite side of the first concave surface 1742, and a bearing socket 1745 formed on the first concave surface 1742. The threaded shaft 1745 may be coupled to the hemispherical surface 1743 of the semi-spherical joint 1741, and it may protrude from the base 1733 of the cradle 1704. When the locking members 1722 of the cylindrical adaptor 1704 are deployed, the semi-spherical joint 1741 may be retained within the cylindrical space defined by the cradle 1704.

The bearing socket 1745 may be used for receiving a drilling force to drive the threaded shaft 1745 into a particularly bone segment, thereby anchoring the cradle 1704 to that bone segment. After being anchored, the base 1733 of the cradle 1704 may engage and pivot the hemispherical surface 1743 of the semi-spherical joint 1741, such that the threaded shaft 1745 may have the first multi-axle movement (See FIG. 13C) about the cradle axis. In one embodiment, the base 1733 may include a convex pivot ring (not shown), which may be used for pivoting the hemispherical surface 1743 of the semi-spherical joint 1741. In another embodiment, the base 1733 may pivot the hemispherical surface 1743 of the semi-spherical joint 1741 via the cylindrical adaptor 1706, which may have one or more convex pivot rings 1724.

The first concave surface 1742 of the semi-spherical joint 1741 may be used for receiving, contacting, and engaging the spherical joint 1750. As such, the spherical joint 1750 may move freely around the first concave surface 1742. The free movement of the spherical joint 1750 may facilitate part of the second multi-axle movement since the semi-spherical joint 1741 may become an integral part of the cradle 1704.

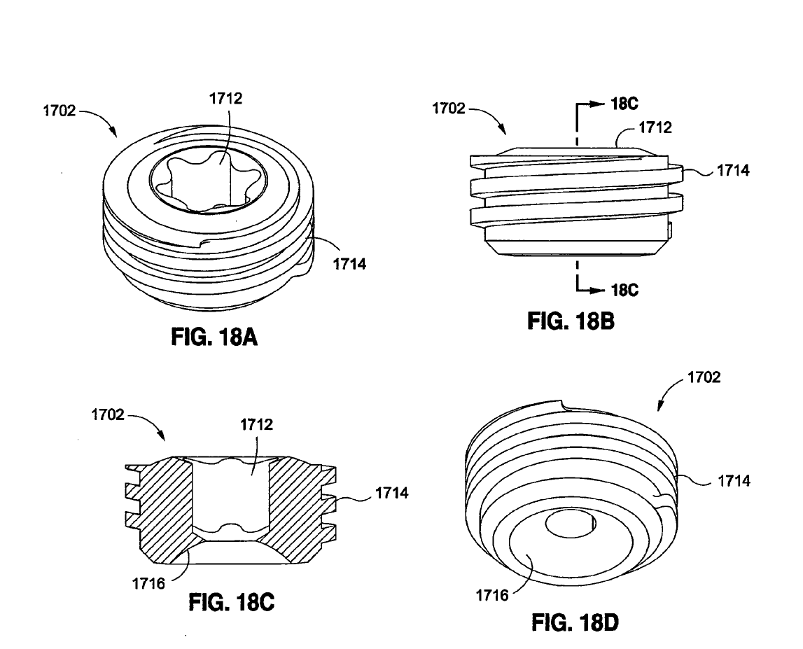

Generally, as shown in FIG. 17C and FIGS. 18A-18D, the set screw 1702 may have a socket 1712, a threaded side wall 1714, and a second concave surface 1716. Particularly, the socket 1712 may be used for receiving a locking force, the second concave surface 1716 may be positioned on the opposite side of the socket 1712, and the threaded side wall 1714 may be coupled between the socket 1712 and the second concave surface 1716.

To secure the spherical joint 1750, the threaded side wall 1714 may engage the inner threaded surface 1732 of the cradle 1704 until the second concave surface 1716 makes contact with the spherical joint 1750. At that point, the spherical joint 1750 may move freely around the second concave surface 1716. The free movement of the spherical joint may facilitate part of the second multi-axle movement since the set screw 1712 may become an integral part of the cradle 1704. Together, the first and second concave surfaces 1742 and 1716 may cooperatively engage the spherical joint 1750, such that the cradle 1704 may achieve the second multi-axle movement about the spherical joint 1750.

To lock the spherical joint 1750 in position, the threaded side wall 1714 of the set screw 1702 may convert the locking force received from the socket 1712 to a compression force. The second concave surface 1716 may apply the compression force against the spherical joint 1750. Moreover, the compression force may be redirected to the base 1733 of the cradle 1704, which may respond by generating a reaction force. Eventually, the first concave surface 1742 of the semi-spherical joint 1741 may redirect the reaction force against the spherical joint 1750. Together, the compression force and the reaction force may cooperate with each other, and they may cause a simultaneous reduction of the first and second multi-axle movements. Accordingly, the spherical joint 1750 may be locked in a particular position within the cradle 1704.

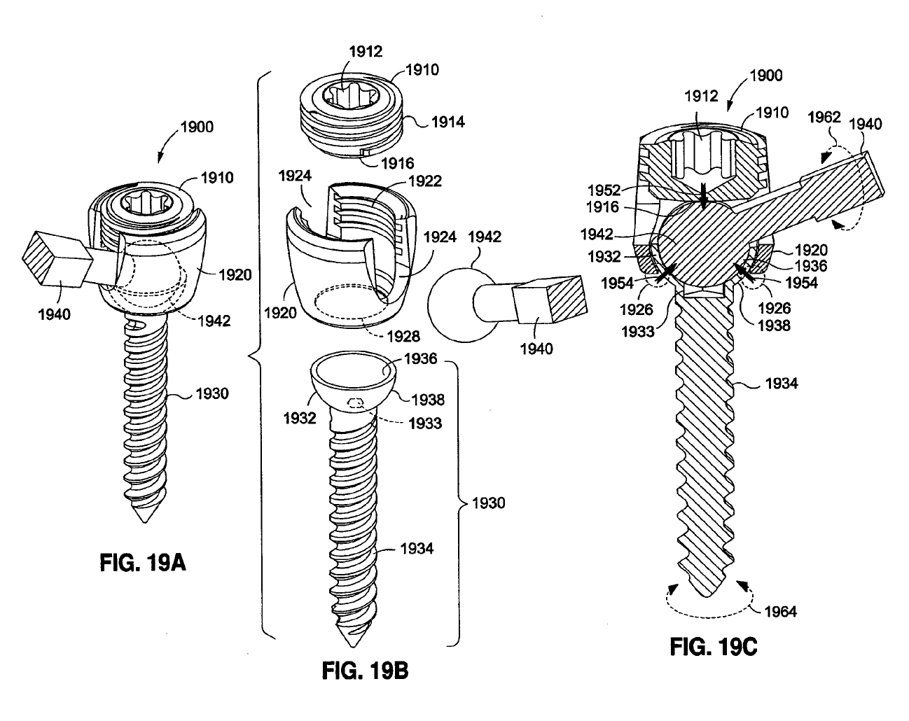

FIGS. 19A-19C show various views of another joint receiving (JR) pedicle screw 1900 according to another embodiment of the present invention. The JR pedicle screw 1900 may include a set screw 1910, a cradle 1920, and a screw member 1930. The cradle 1920 may enclose part of the screw member 1930, and it may receive and secure the spherical joint 1942 after being engaged by the set screw 1910. The spherical joint 1942 may be coupled to the flat end member 1940, which may be part of the Real-X, Real-O, or Real-XO cross connector.

Referring to FIG. 19B, which shows the exploded view of the JR pedicle screw 1900, the screw member 1930 may include a joint holder 1932 and a threaded shaft 1934 coupled to the joint holder 1932. The joint holder 1932 may have a concave inner surface 1936 and a convex outer surface 1938. Initially, the joint holder 1932 may be received by the cradle 1920, while the threaded shaft 1934 may protrude from the base of the cradle 1920. The cradle 1920 may be anchored to a spinal bone segment by the screw member 1930. Particularly, the screw member 1930 may have a bearing socket 1933 for receiving a surgical ranch, which may drive the threaded shaft 1934 into the spinal bone segment around the pedicle region. Because the cradle 1920 is engaged by the convex outer surface 1938 of the joint holder 1932, the cradle 1920 may be anchored to the spinal bone segment via the threaded shaft 1934.

After being anchored to the spinal bone segment, the cradle 1920 may move around the joint holder 1932. As shown in FIG. 19C, the cradle 1920 may have a convex pivot ring 1926 located adjacent to the base opening 1928. The convex pivot ring 1926 may be used for pivoting the outer convex surface 1938 of the joint holder 1932. In relation to the cradle 1920, the threaded shaft 1934 may have a first multi-axial movement 1964. The size of the base opening 1928 of the cradle 1920 may limit the range of the first multi-axial movement 1964.

The cradle 1920 may receive the spherical joint 1942. After the spherical joint 1942 is positioned within the cradle 1920, the flat end member 1940 may protrude from the cradle 1920 via one of the receiving ports 1924. The concave inner surface 1936 of the joint holder 1932 may be used for contacting the spherical joint 1942. As such, the spherical joint 1942 may move around the concave inner surface 1936.

The set screw 1910 may have a bearing socket 1912, a contact surface 1916 positioned on the opposite side of the bearing socket 1912, and a threaded side wall 1914 coupled between the bearing socket 1912 and the contact surface 1916. The bearing socket 1912 may be used for receiving a locking force applied by a surgical ranch. The threaded side wall 1914 may engage the inner threaded side wall 1922 of the cradle 1920 while the bearing socket 1912 is receiving the locking force. As the set screw 1910 descends into the cradle 1920, the contact surface 1916 may contact and engage the spherical joint 1942. The contact surface 1916 may be flat, convex, or concave. In one embodiment, the contact surface 1916 may be convex, which may establish a single contact point with the spherical joint 1942. In another embodiment, the contact surface 1916 may be concave, which may establish a plurality of contact points with the spherical joint 1942.

The contact surface 1916 may cooperate with the concave inner surface 1936 to allow the spherical joint 1942 to freely rotate within the cradle 1920. Accordingly, the flat end member 1940 may have a second multi-axle movement 1940 in relative to the cradle 1920. The size of the receiving ports 1924 may limit the range of the second multi-axle movement 1962.

When the threaded side wall 1914 of the set screw 1910 is substantially engaged to the inner threaded side wall 1922 of the cradle 1920, the locking force may be converted to a compression force 1952. The contact surface 1916 of the set screw 1910 may apply the compression force 1952 against the spherical joint 1942. The compression force 1952 may be redirected to the base of the cradle 1920. As a result, the convex pivot ring 1926 of the cradle 1920 may apply a reaction force 1954 along a circular path and against the outer convex surface 1938 of the joint holder 1932. In turn, the joint holder 1932 may redirect the reaction force 1954 to the spherical joint 1942.

The compression force 1952 may cooperate with the reaction force 1954 to substantially restrain the relative movements among the spherical joint 1942, the joint holder 1932, and the cradle 1920. By tightening the set screw 1910 into the cradle 1920, the first and second multi-axle movements 1964 and 1962 may be simultaneously reduced and suspended. To prevent the joint holder 1932 from sliding within the cradle 1920, the convex pivot ring 1926 may be depressible, the feature of which may increase the friction between the outer convex surface 1938 and the base section of the cradle 1920. To prevent the spherical joint 1940 from moving along the joint holder 1932, the inner concave surface 1936 may include one or more depressible bumps, rings, or protrusions, which may be used for increasing the friction between the inner concave surface 1936 and the spherical joint 1942. Compared to conventional pedicle screws, the JR pedicle screw 1900 may be easier to manufacture and assemble because it has fewer components and installation steps.

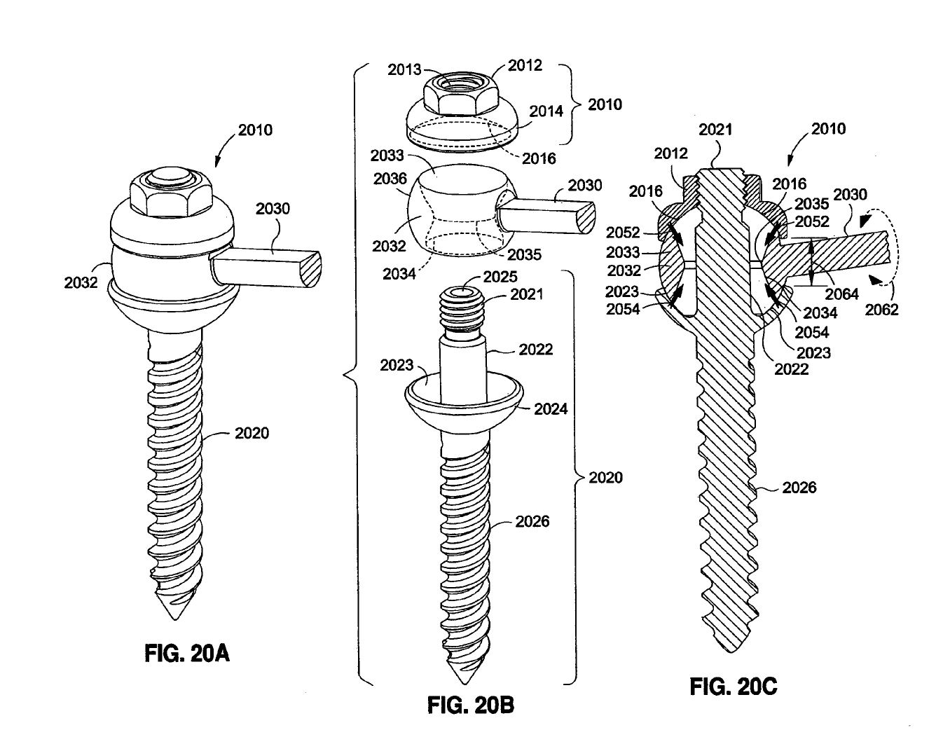

FIGS. 20A-20C show various views of an alternative joint receiving (JR) pedicle screw 2000 according to an alternative embodiment of the present invention. Generally, the alternative JR pedicle screw 2000 may include a cap member 2010 and a base member 2020. The alternative JR pedicle screw 2000 may be used in conjunction with a cross connector having a spherical ring joint 2032, which may be connected to the flat end member 2030 of the cross connector.

The spherical ring joint 2032 may serve similar functions as the spherical joints as discussed in FIG. 13B, and it may be combined with the Real-X, Real-O, and/or Real-XO cross connectors. Moreover, the spherical ring joint 2032 may include a double conical channel (hour-glass channel) along one of its central axes. The double conical channel may have a first inner conical surface 2033, a second inner conical surface 2034, and an inner neck 2035 connecting the first and second inner conical surfaces 2033 and 2034. The spherical ring joint 2032 may have a toroidal mid-section 2036, which may have a convex surface similar to the middle section of a sphere.

The base member 2020 may include a threaded head 2021, a pivot pole 2022 coupled to the threaded head 2021, a first (bottom) joint holder 2024 peripherally coupled to the pivot pole 2022, and a threaded shaft 2026 coupled to the pivot pole 2022. The threaded head 2021 may include a bearing socket 2025, which may be driven by a surgical ranch. As such, the threaded shaft 2026 may be driven into a spinal bone segment and thereby anchoring the base member 2020 to the spinal bone segment.

After being anchored, the base member 2020 may receive the spherical ring joint 2032. Particularly, the double conical channel of the spherical ring joint 2032 may be penetrated by the pivot pole 2022 of the base member 2020. The first joint holder 2024 of the base member 2020 may have a first concave surface 2023 for contacting the toroidal section 2036 of the spherical ring joint 2032. The spherical ring joint 2032 may move around the first concave surface 2023, such that the flat end member 2030 may have a wide range of relative movement with respect to the threaded shaft 2026.

After receiving the spherical ring joint 2036, the base member 2020 may be engaged by the cap member 2010. Particularly, the cap member 2010 may have a set screw 2012 and a second (top) joint holder 2014 coupled to the set screw 2012. The set screw 2012 may have an inner threaded section 2013 for engaging the threaded head 2021 of the base member 2020. The second joint holder 2014 may contact the spherical ring joint 2032 as the set screw 2012 is further engaged to the screw head 2021.

The set screw 2012 and the threaded head 2021 may cooperatively lock the second joint holder 2014 at a particular position, thereby retaining the spherical ring joint 2032 in between the first and second concave surfaces 2023 and 2016. As such, the spherical ring joint 2023 may be anchored to the spinal bone segment.

The first and second concave surfaces 2023 and 2016 may engage the toroidal mid-section 2036 of the spherical ring joint 2032, thereby allowing the spherical ring joint 2032 to freely rotate. Moreover, the first and second inner conical surfaces 2033 and 2034 may facilitate a wide range of movement between the spherical ring joint 2032 and the pivot pole 2022. As such, the flat end member 2030 may have a multi-axle movement 2062 along a circular space 2064, which may be defined between the first and second joint holders 2024 and 2014.

When the threaded wall 2013 of the set screw 2012 is substantially engaged to the threaded head 2021, the second concave surface 2016 may assert a compression force 2052 against the spherical ring joint 2032. Particularly, the compression force 2052 may be applied along a circular path on the toroidal mid-section 2036. The compression force 2052 may be redirected to the first concave surface 2023. In response, the first concave surface 2023 may generate a reaction force 2054, which may be applied along another circular path on the toroidal mid-section 2036.

Together, the compression force 2052 may cooperate with the reaction force 2054 to substantially restrain the relative movement between the spherical ring joint 2032 and the pivot pole 2022. As a result, the multi-axle movements 2062 may be reduced and suspended in one single step. To prevent the spherical ring joint 2032 from moving along the first and second concave surfaces 2023 and 2016, each of the first and second concave surfaces 2023 and 2016 may include one or more depressible bumps, rings, or protrusions, which may be used for increasing the friction between the spherical ring joint 2032 and the first and second concave surfaces 2023 and 2016. Compared to conventional pedicle screws, the alternative JR pedicle screw 2000 may be easier and less costly to manufacture and assemble because it has fewer components and installation steps.

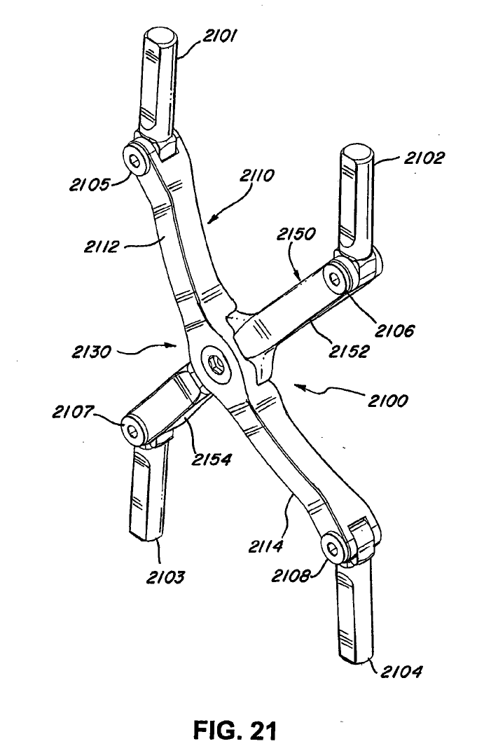

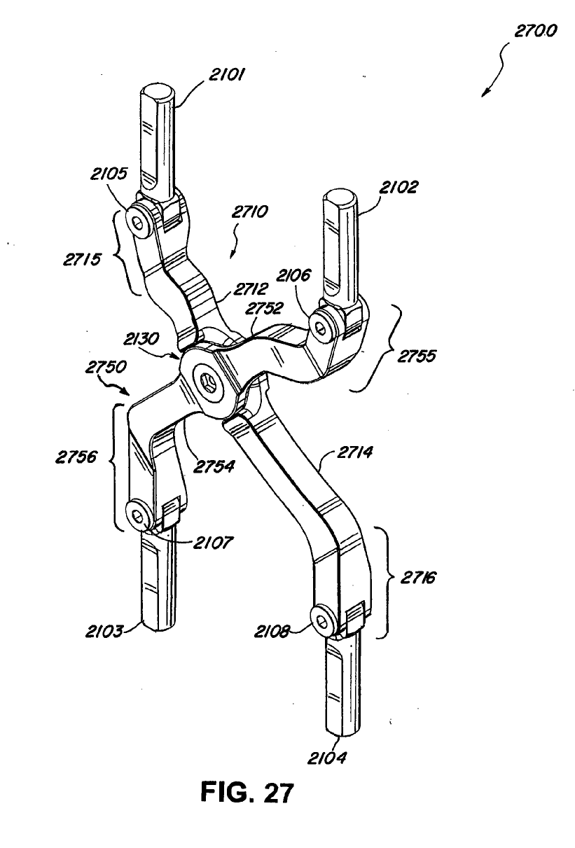

The discussion now turns to two alternative embodiments with enhanced stress redistribution. The first alternative embodiment encompasses a Real-X cross connector with an enhanced stress redistribution structure and a fortified pivoting means. Similarly, the second alternative embodiment encompasses a Real-X cross connector with an enhanced stress redistribution structure and a fortified pivoting means, as well as a spinous-process adaptive contour for fitting around the spinous process of a patient. In the following sections, FIGS. 21-26 will disclose the structural and functional features of first alternative embodiment, while FIGS. 27-32 will disclose the structural and functional features of the second alternative embodiment.

FIG. 21 shows a perspective view of an RXB cross connector 2100 according to a first alternative embodiment of the present invention. The RXB cross connector 2100 may be used for stabilizing and protecting one or more fixation levels of spinal bone segments. In practice, the RXB cross connector 2100 may be adjustably equipped with several conventional rod segments, such as a first rod 2101, a second rod 2102, a third rod 2103, and a fourth rod 2104. The RXB cross connector 2100 may be affixed to two or more spinal bone segments by anchoring the conventional rod segments (e.g., the first rod 2101, the second rod 2102, the third rod 2103, and/or the fourth rod 2104) to the pedicle areas of these spinal bone segments. For example, one or more pedicle screws can be used as anchoring devices for anchoring the conventional rod segments to the pedicle areas of the spinal bone segments.

The RXB cross connector 2100 may include a first connector (top link) 2110, a second connector (bottom link 2150), and a pivot joint 2130. In order to form an X-shaped bridge across the targeted spinal bone segments, the pivot joint 2130 may pivot the mid section of the first connector 2110 against the mid section of the second connector 2150. In one implementation, for example, the pivot joint 2130 may be an integral part of the first connector 2110 and the second connector 2150. In another implementation, for example, the pivot joint 2130 may be a separate part of the first connector 2110 and/or the second connector 2150. In yet another implementation, for example, the pivot joint 2130 may be partially integrated with the first connector 2110 and/or the second connector 2150.

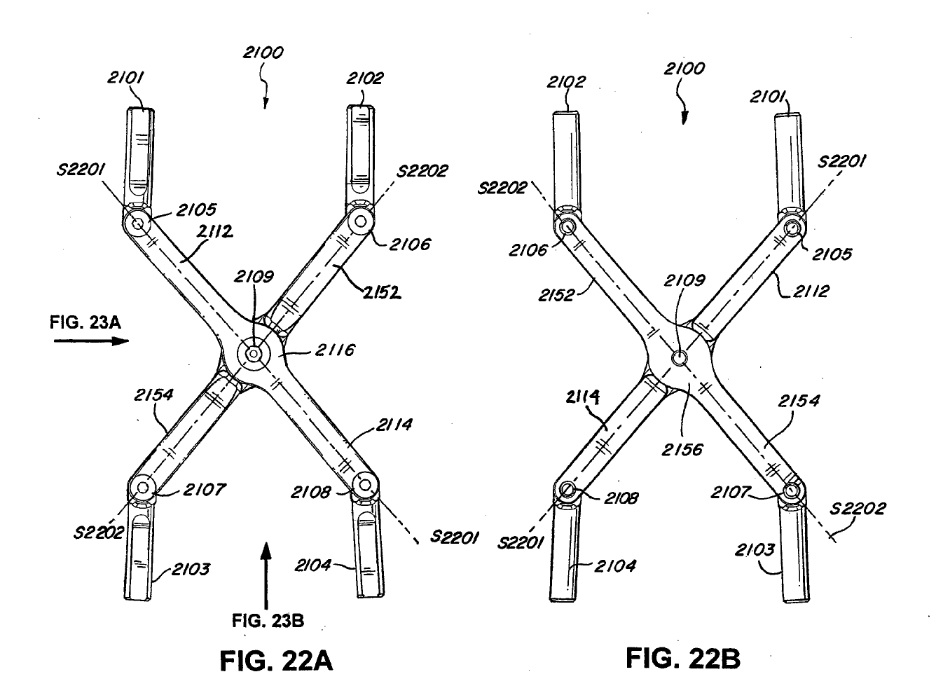

FIGS. 22A and 22B show a front view and a back view of the RXB cross connector 2100, the first connector 2110 may include a first arm 2112, a third arm 2114, and an upper platform 2116, while the second connector 2150 may include a second arm 2152, the fourth arm 2154, and a lower platform 2156. As discussed herein, the numerical terms, such as “first,” “second,” “third,” and “fourth,” are relative terms such that they may be used interchangeably. Moreover, as discussed herein, the positioning terms, such as “upper,” “lower,” “top,” and, “bottom,” are relative terms such that they may also be used interchangeably.

The first arm 2112 may be pivotally connected to the first rod 2101 via a first screw 2105. When the first screw 2105 is not fastened, the first rod 2101 may have a range of radial movement about the first screw 2105. When the first screw 2105 is substantially fastened, the first rod 2101 may be tightly connected to the first arm 2112 such that the relative motion between the first rod 2101 and the first arm 2112 may be substantially restricted.

The third arm 2114 may be pivotally connected to the fourth rod 2104 via a fourth screw 2108. When the fourth screw 2108 is not fastened, the fourth rod 2104 may have a range of radial movement about the fourth screw 2108. When the fourth screw 2108 is substantially fastened, the fourth rod 2104 may be tightly connected to the third arm 2114 such that the relative motion between the fourth rod 2104 and the third arm 2114 may be substantially restricted.

The second arm 2152 may be pivotally connected to the second rod 2102 via a second screw 2106. When the second screw 2106 is not fastened, the second rod 2102 may have a range of radial movement about the second screw 2106. When the second screw 2106 is substantially fastened, the second rod 2102 may be tightly connected to the second arm 2152 such that the relative motion between the second rod 2102 and the second arm 2152 may be substantially restricted.

The fourth arm 2154 may be pivotally connected to the third rod 2103 via a third screw 2107. When the third screw 2107 is not fastened, the third rod 2103 may have a range of radial movement about the third screw 2107. When the third screw 2107 is substantially fastened, the third rod 2103 may be tightly connected to the fourth arm 2154 such that the relative motion between the third rod 2103 and the fourth arm 2154 may be substantially restricted.

The upper platform 2116 may connect the first arm 2112 to the third arm 2114, such that the first arm 2112 and the third arm 2114 may form a contiguous arc segment along a first reference plane S2201. Similarly, the lower platform 2156 may connect the second arm 2152 to the fourth arm 2154, such that the second arm 2152 and the fourth arm 2154 may form another contiguous arc segment along a second reference plane S2202. When viewed from the top and the bottom of the RXB cross connector 2100, these two contiguous arc segments may appear as two straight and elongated members crossing each other to form an X-shaped protection bridge. Hence, the first reference plane S2201 may intersect with the second reference plane S2202 along a center axis (pivot axis) Ax.

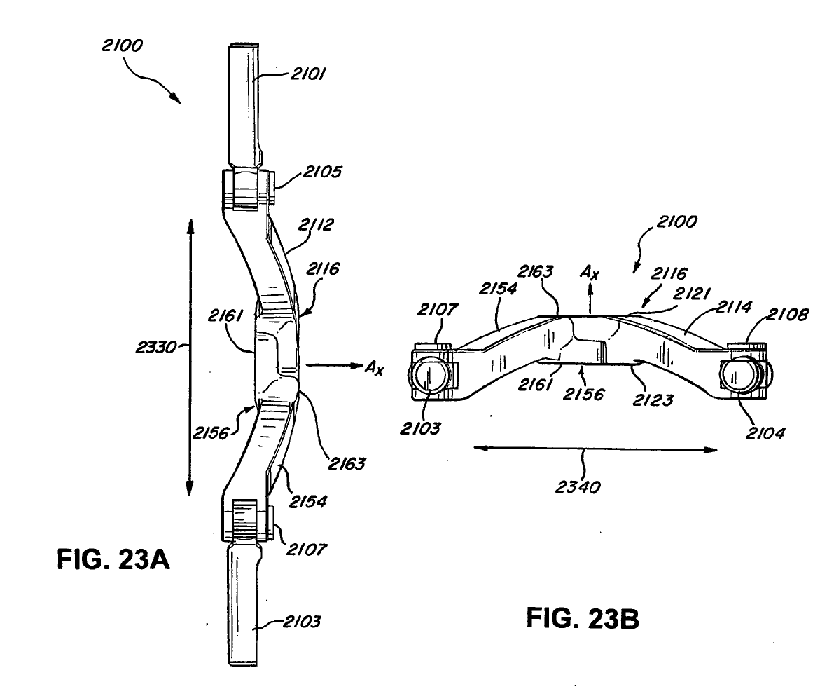

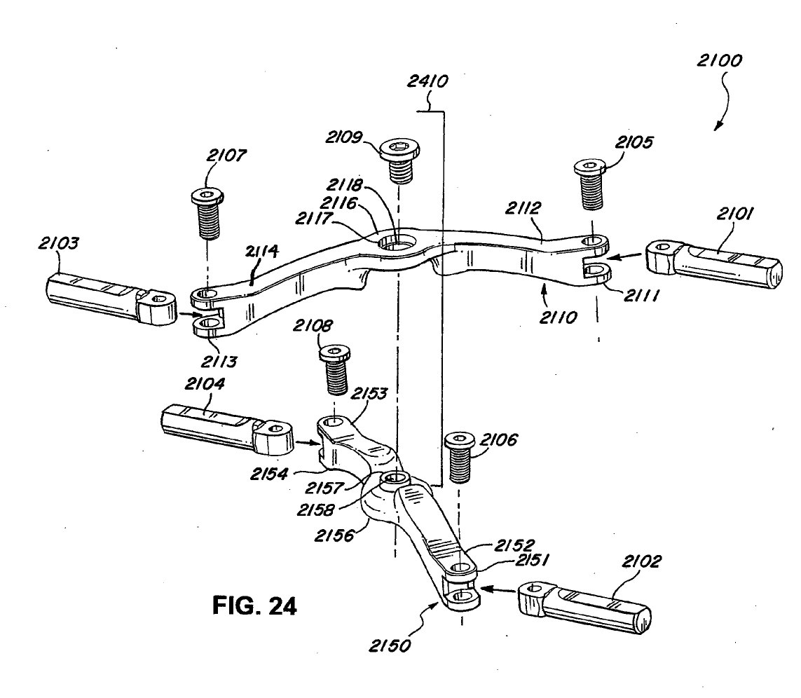

As shown in FIGS. 23A-23B, the upper platform 2116 may interpose the lower platform 2156 along and about the center axis Ax. The lower platform 2156 may include one or more components for engaging the upper platform 2116. Such an engagement may provide a pivoting means for the RXB cross connector 2100, thereby allowing the RXB cross connector 2100 to have an adjustable length 2330 and an adjustable width 2340. This aspect of the first alternative embodiment will be further illustrated and discussed in FIG. 24.

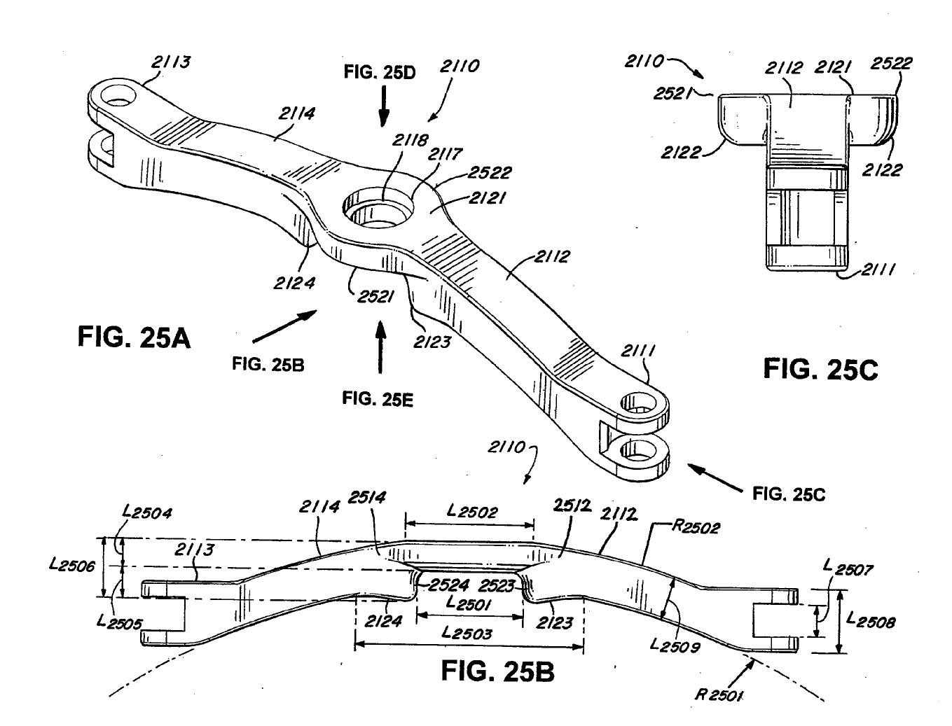

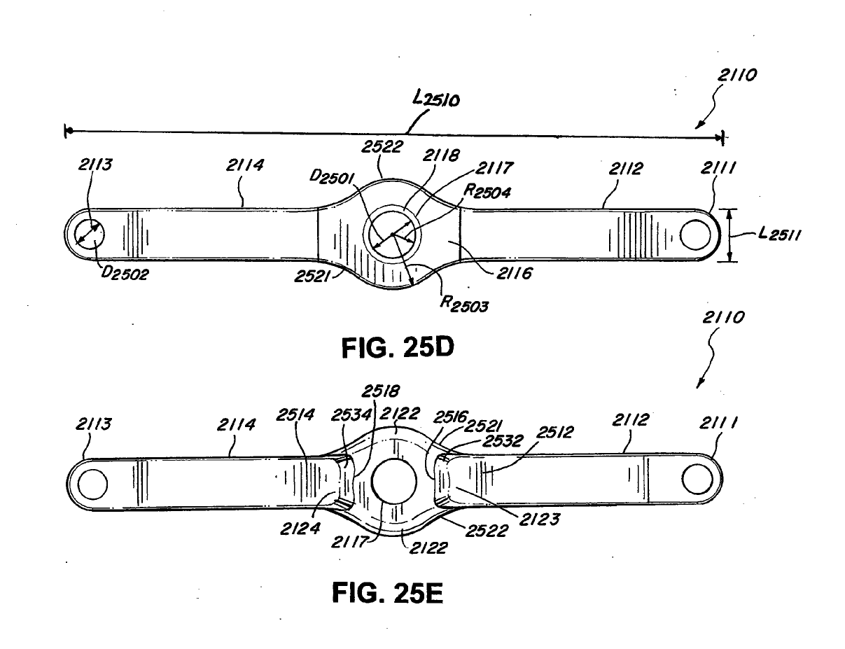

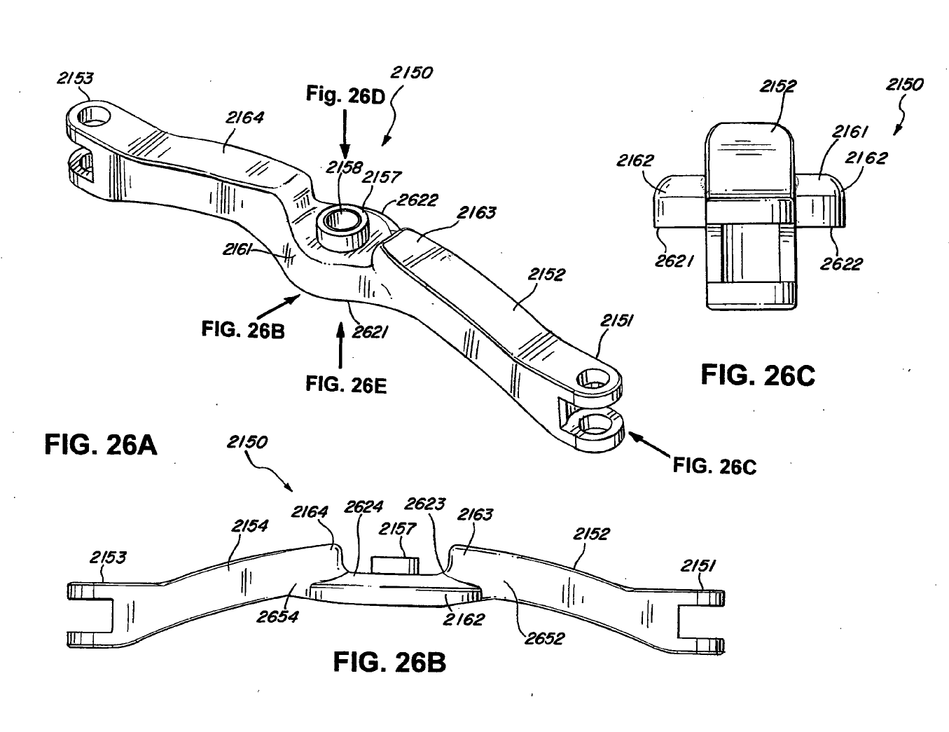

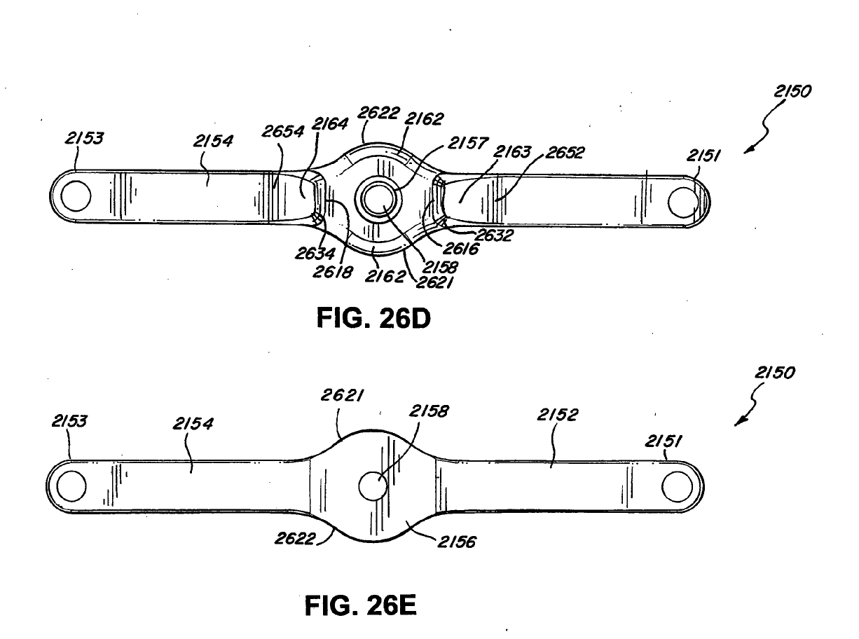

Moreover, the upper platform 2116 may establish a complementary relationship with the lower platform 2156. In one configuration, the upper platform 2116 may include an upper plate (top plate) 2121 and one or more lower brackets, such as the lower bracket 2123. The lower brackets (e.g., the lower bracket 2123) may join the upper plate 2121 at its edges to form one or more upper (upside-down) valleys, the detail of which will be further illustrated and discussed in FIG. 25B. In another configuration, the lower platform 2156 may include a lower plate (bottom plate) 2161 and one or more upper brackets, such as the upper bracket 2163. The upper brackets (e.g., the upper bracket 2163) may join the lower plate 2161 at its edges to form one or more lower valleys, the detail of which will be further illustrated and discussed in FIG. 26B.

Because the upper platform 2116 and the lower platform 2156 are complementarily configured and positioned, the upper plate 2121 may be snugly fitted within the lower valley while the lower plate 2161 may be snugly fitted within the upper valley. The upper valley may help redistribute and redirect the mechanical stress received by the bottom plate 2161. Similarly, the lower valley may help redistribute and redirect the mechanical stress received by the upper plate 2121. Because of the mutual stress redistribution and redirection, the upper platform 2116 may cooperate with the lower platform 2156 to enhance the rigidity and stability of the RXB cross connector 2100. This functional feature of the RXB cross connector 2100 will be further illustrated discussed in FIGS. 25A-25E and 26A-26E.

Referring to FIG. 24, the RXB cross connector 2100 may include several pivoting points. The first pivoting point, for example, may be located at a distal end 2111 of the first arm 2112. When the first screw 2105 partially engages the first distal end 2111 and the first rod 2101, the first rod 2101 may freely rotate about the shaft of the first screw 2105. When the first screw 2105 substantially engages the first distal end 2111, the first screw 2105 may help tighten the lips of the first distal end 2111, thereby substantially restricting the movement of the first rod 2101. As such, the first rod 2101 can be locked in a particular position with respect to the first distal end 2111 of the first arm 2112.

The second pivoting point, for example, may be located at a distal end 2151 of the second arm 2152. When the second screw 2106 partially engages the second distal end 2151 and the second rod 2102, the second rod 2102 may freely rotate about the shaft of the second screw 2106. When the second screw 2106 substantially engages the second distal end 2151, the second screw 2106 may help tighten the lips of the second distal end 2151, thereby substantially restricting the movement of the second rod 2102. As such, the second rod 2102 can be locked in a particular position with respect to the second distal end 2151 of the second arm 2152.

The third pivoting point, for example, may be located at a distal end 2113 of the third arm 2114. When the third screw 2107 partially engages the third distal end 2113 and the third rod 2103, the third rod 2103 may freely rotate about the shaft of the third screw 2107. When the third screw 2107 substantially engages the third distal end 2113, the third screw 2107 may help tighten the lips of the third distal end 2113, thereby substantially restricting the movement of the third rod 2103. As such, the third rod 2103 can be locked in a particular position with respect to the third distal end 2113 of the third arm 2114.

The fourth pivoting point, for example, may be located at a distal end 2153 of the fourth arm 2154. When the fourth screw 2108 partially engages the fourth distal end 2153 and the fourth rod 2104, the fourth rod 2104 may freely rotate about the shaft of the fourth screw 2108. When the fourth screw 2108 substantially engages the fourth distal end 2153, the fourth screw 2108 may help tighten the lips of the fourth distal end 2153, thereby substantially restricting the movement of the fourth rod 2104. As such, the fourth rod 2104 can be locked in a particular position with respect to the fourth distal end 2153 of the fourth arm 2154.

The distal ends (e.g., the first distal end 2111, the second distal end 2151, the third distal end 2113, and/or the fourth distal end 2153) may define the reach of the RXB cross connector 2100. The pivoted rods (e.g., the first rod 2101, the second rod 2102, the third rod 2103, and/or the fourth rod 2104) may provide the anchoring points for the RXB cross connector 2100.

Generally, the upper platform 2116 and the lower platform 2156 may each include one or more physical structures for effectuating the pivoting therebetween. In one configuration, for example, the lower platform 2156 may include a hollow pole 2157 with a threaded interior surface 2158, while the upper platform 2116 may include a top opening 2117 with a top stopper 2118. To engage the upper platform 2116 to the lower platform 2156, the hollow pole 2157 may be inserted into the top opening 2117. After the insertion, the first connector 2110 may be free to rotate about the pivot axis Ax and with respect to the second connector 2150. A set screw 2109 may be used for securing the upper platform 2116 against the lower platform 2156.

When the set screw 2109 partially engages the threaded interior surface 2158 of the hollow pole 2157, the first connector 2110 may freely rotate about the pivot axis Ax while the upper platform 2116 remains substantially in contact with the lower platform 2156. When the set screw 2109 substantially engages the threaded interior surface 2158, the set top portion of the set screw 2109 may push downward and against the top stopper 2118 of the upper platform 2116. Simultaneously, the threaded shaft of the set screw 2109 may pull the lower platform 2156 upward and against upper platform 2116. As a result, a pair of action and reaction forces may be asserted against the inner surfaces of the upper platform 2116 and the lower platform 2156. The action and reaction forces may substantially restrict the relative rotational movement between the upper platform 2116 and the lower platform 2156, thereby locking the RXB cross connector 2100 into a particular angle. Together, the set screw 2109, the upper platform 2116, and the lower platform 2156 may form pivoting group 2410 for providing a pivoting means for the RXB cross connector 2100.

The discussion now turns to the structure and functional features of the first connector (top link) 2110 and the second connector (bottom link) 2150 of the RXB cross connector 2100. Referring to FIGS. 25A-25E, the upper platform 2116 may be subdivided into several sections, including but not limited to, a top plate 2121, a first top side wall 2512, and a second top side wall 2514. The first top side wall 2512 may connect the top plate 2121 to the first arm 2112, while the second top side wall 2514 may connect the top plate 2121 to the third arm 2114.

Generally, the top plate 2121 may have a radius that is much larger than a width of the first arm 2112 and/or the third arm 2114. The first top side wall 2512 may provide a geometric transition from the first arm 2112 to the top plate 2121, while the second top side wall 2514 may provide another geometric transition from the third arm 2114 to the top plate 2121. Such geometric transitions may help reduce the stress concentration at the junction of the top plate 2121 and the first arm 2112, as well as the stress concentration at the junction of the top plate 2121 and the third arm 2114.

Referring to FIGS. 26A-26E, the lower platform 2156 may be subdivided into several sections, including but not limited to, a bottom plate 2161, a first bottom side wall 2652, and a second bottom side wall 2654. The first bottom side wall 2652 may connect the bottom plate 2161 to the second arm 2152, while the second bottom side wall 2654 may connect the bottom plate 2161 to the fourth arm 2154.

Similar to the top plate 2121, the bottom plate 2161 may have a radius that is much larger than a width of the second arm 2152 and/or the fourth arm 2154. The first bottom side wall 2652 may provide a geometric transition from the second arm 2152 to the bottom plate 2161, while the second bottom side wall 2654 may provide another geometric transition from the fourth arm 2154 to the bottom plate 2161. Such geometric transitions may help reduce the stress concentration at the junction of the bottom plate 2161 and the second arm 2152, as well as the stress concentration at the junction of the bottom plate 2161 and the fourth arm 2154.

Next, the structural and functional features of the upper platform 2116 will be discussed in conjunction with those of the lower platform 2156. The top plate 2121 may have a first upper bell-shaped ridge (bow-shaped ridge) 2521 and a second upper bell-shaped ridge (bow-shaped ridge) 2522. Each of the bell-shaped ridges may have an upper convex edge 2122. Similarly, the bottom plate 2161 may have a first lower bell-shaped ridge (bow-shaped ridge) 2621 and a second lower bell-shaped ridge (bow-shaped ridge) 2622. Each of the bell-shaped ridges may have a lower convex edge 2162.

Each of the top side walls may include a lower bracket. Developing from the upper platform 2116, the first top side wall 2512 may include a first lower bracket 2123 while the second top side wall 2514 may include a second lower bracket 2124. The first lower bracket 2123 may be opposing the first second lower bracket 2124 in such a manner that they can form an upper (inverse) valley with the top plate 2121. The upper valley may align with the first reference plane S2201, and it may define a receiving cradle for embracing the bottom plate 2162.

More specifically, the first lower bracket 2123 may have a first lower ventral concave surface 2532 facing away from the first arm 2112, while the second lower bracket 2124 may have a second lower ventral concave surface 2534 facing away from the third arm 2114. The first lower ventral concave surface 2532 may define a first lower vertical concave contour 2523 and a first lower horizontal concave contour 2516. Similarly, the second lower ventral concave surface 2534 may define a second lower vertical concave contour 2524 and a second lower horizontal concave contour 2518. On one hand, the first lower vertical concave contour 2523 and the second lower vertical concave contour 2524 may be parallel with the first reference plane S2201. On the other hand, the first lower horizontal concave contour S516 and the second lower horizontal concave contour 2518 may be perpendicular with the first reference plane S2201.

The first lower vertical concave contour 2523 and the second lower vertical concave contour 2524 may have a complementary arrangement with the lower convex edges 2162 of the first lower bell-shaped ridge 2621 and the second lower bell-shaped ridge 2622. As such, the lower vertical concave contours (e.g., the first lower vertical concave contour 2523 and/or the second lower vertical concave contour 2524) may fit with the lower convex edges (e.g., the lower convex edges 2122 of the first lower bell-shaped ridge 2621 and the second lower bell-shaped ridge 2622) along an orientation that is parallel with the first reference plane S2201.

The first lower horizontal concave contour 2516 and the second lower horizontal concave contour 2518 may have a complementary arrangement with the first lower bell-shaped ridge 2621 and the second lower bell-shaped ridge 2622. As such, the lower horizontal concave contours (the first lower horizontal concave contour 2516 and the second lower horizontal concave contour 2518) may fit with the lower bell-shaped ridges (e.g., the first lower bell-shaped ridge 2621 and the second lower bell-shaped ridge 2622) along an orientation that is perpendicular to the first reference plane S2201. Because of these various complementary arrangements, the bottom plate 2156 may fit snugly within the upper (inverse) valley.

The lower platform 2156 may have a similar configuration as the upper platform 2116. For instance, each of the bottom side walls may include a lower bracket. Developing from the lower platform 2156, the first bottom side wall 2652 may include a first upper bracket 2163 while the second bottom side wall 2654 may include a second upper bracket 2164. The first upper bracket 2163 may be opposing the first second upper bracket 2164 in such a manner that they can form a lower valley with the bottom plate 2161. The lower valley may align with the second reference plane S2202, and it may define a receiving cradle for embracing the top plate 2121.

More specifically, the first upper bracket 2163 may have a first upper ventral concave surface 2632 facing away from the second arm 2152, while the second upper bracket 2164 may have a second upper ventral concave surface 2634 facing away from the fourth arm 2154. The first upper ventral concave surface 2632 may define a first upper vertical concave contour 2623 and a first upper horizontal concave contour 2616. Similarly, the second upper ventral concave surface 2634 may define a second upper vertical concave contour 2624 and a second upper horizontal concave contour 2618. On one hand, the first upper vertical concave contour 2623 and the second upper vertical concave contour 2624 may be parallel with the second reference plane S2202. On the other hand, the first upper horizontal concave contour 2616 and the second upper horizontal concave contour 2618 may be perpendicular with the second reference plane S2202.

The first upper vertical concave contour 2623 and the second upper vertical concave contour 2624 may have a complementary arrangement with the upper convex edges 2122 of the first upper bell-shaped ridge 2121 and the second upper bell-shaped ridge 2122. As such, the upper vertical concave contours (e.g., the first upper vertical concave contour 2623 and/or the second upper vertical concave contour 2624) may fit with the upper convex edges (e.g., the upper convex edges 2122 of the first upper bell-shaped ridge 2121 and the second upper bell-shaped ridge 2122) along an orientation that is parallel with the second reference plane S2202.

The first upper horizontal concave contour 2616 and the second upper horizontal concave contour 2618 may have a complementary arrangement with the first upper bell-shaped ridge 2121 and the second upper bell-shaped ridge 2122. As such, the upper horizontal concave contours (the first upper horizontal concave contour 2616 and the second upper horizontal concave contour 2618) may fit with the upper bell-shaped ridges (e.g., the first upper bell-shaped ridge 2121 and the second upper bell-shaped ridge 2122) along an orientation that is perpendicular to the second reference plane S2202. Because of these various complementary arrangements, the top plate 2156 may fit snugly within the lower valley.

The interposing of the upper valley with the top plate 2121, as well as the interposing of the lower valley with the bottom plate 2121, may provide at least two benefits. First, the concave sections of the valleys may properly absorb, redirect, and/or redistribute the stress lines built up in the convex edges of the respective plates. Second, the concave sections of the valleys may provide one or more smooth contact surfaces for restricting the lateral movements of the respective plates. Such a restriction may minimize the wearing of the joint segment (e.g., the total contact surfaces of the first connector 2110 and the second connector 2150) while enhancing the stability and rigidity of RXB cross connector 2100.

The discussion now turns to various dimensions of the first connector 2110 and the second connector 2150. Referring to FIG. 25B, the upper valley may have a valley width L2501, the lower brackets 2123 and 2124 may have a bracket width L2502, and the upper platform 2116 may have a platform length L2503. In one configuration, the valley width L2501 may be about 12.08 mm, the bracket width L2502 may be about 15.03 mm, and the platform length L2503 may be about 25.07 mm. The top plate 2121 may have a plate thickness L2504 and the upper valley may have a valley height L2505. In one configuration, the plate thickness L2504 may be about 3.25 mm, and the valley height L2505 of about 3.25 mm as well. Accordingly, the upper platform 2116 may have a total platform height L2506 of about 6.5 mm.

Each of the first arm 2112 and the third arm 2114 may have an arm thickness L2509, an inner curvature 82501, and an outer curvature 82502. In one configuration, the arm thickness L2509 may be about 4 mm, the inner curvature 82501 may have a radius of about 74 mm, and the outer curvature 82502 may have a radius of about 75 mm. Each of the first distal end 2111 and the third distal end 2113 may have a distal end height L2507 and an inter-lip space L2507. In one configuration, the distal end height L2507 may be about 7.5 mm, and the inter-lip space may be about 4 mm.

Referring to FIG. 25D, the first connector 2110 may have a connector length L2510 and a connector width L2511. In one configuration, the connector length L2510 may be about 72 mm, and the connector width L2511 may be about 6 mm. Moreover, the top plate may have a plate radius 82503, the top opening 2117 may define an open radius 82504, the top stopper 2118 may define an inner diameter D2501, and the distal ends 2111 and 2113 may each define a pivot opening with a distal diameter D2502. In one configuration, the plate radius 82503 may be about 6.5 mm, the open radius 82504 may be about 3.5 mm, the inner diameter D2501 may be about 5.5 mm, and the distal diameter D2502 may be about 3.5 mm.

The corresponding and/or matching parts of the second connector 2150 may have dimensions that are similar to those of the first connectors 2110. Additionally, the hollow pole 2157 of the lower platform 2156 may have a pole height and a pole diameter. In one configuration, the pole height may range from 1 mm to about 3 mm, while the pole diameter may range from 4 mm to about 6 mm. In another configuration, the pole height may be about 2 mm, and the pole diameter may be about 5.5 mm.

The discussion now turns to the second alternative embodiment, which is directed to an RXC cross connector 2700, the various views of which are shown in FIGS. 27, 28A-28B, 29A-29B, and 30. Generally, the RXC cross connector 2700 may have structure and functional features that are similar to those of the RXB cross connector 2100. In one configuration, for example, the RXC cross connector 2700 may be used for protecting and stabilizing two or more spinal bone segments. The RXC cross connector 2700 may be anchored to the spinal bone segments via several rods (e.g., the first rod 2101, the second rod 2102, the third rod 2103, and/or the fourth rod 2104), each of which may be pivotally connected to the RXC cross connector 2700 by a screw (e.g., the first screw 2105, the second screw 2106, the third screw 2107, or the fourth screw 2108).

In another configuration, for example, the RXC cross connector 2700 may adopt a pivoting means (e.g., the pivot joint 2130) and a stress redistributing mechanism (e.g., the complementary arrangements between the upper platform 2116 and the lower platform 2156) that are essentially the same as the RXB cross connector 2100. One skilled in the art may readily understand and appreciate these similar features by referencing the previous discussion. As such, the detail description of pivoting means and stress redistributing mechanism will not be repeated in the following sections.

Notwithstanding these similar features, the RXC cross connector 2700 may be distinguished from the RXB cross connector 2100 based on the shape of the various arms. Primarily, when viewed from the top or from the bottom, the arms of the RXB cross connector 2100 may form a straight X-shape bridge while the arms of the RXC cross connector 2700 may form a deflected X-shape bridge. The deflected X-shape bridge may provide the benefit of better fitting around the spinous process of the spinal bone segment.

More specifically, each of the arms may have an arm extension that curves away and deviates from the respective reference plane. In one configuration, the first connector (bottom link) 2710 may have a first arm 2712, a third arm 2714 and a lower platform 2156. The lower platform 2156 may connect the first arm 2712 to the third arm 2714 to form a first arc along the first reference plane S2201. The first arm 2712 may have a first arm extension 2715 deviating from the first reference plane S2201. The first arm extension 2715 may form a first (left) slanted V-shape strip protruding outwardly from the first reference plane S2201. The third arm 2714 may have a third arm extension 2716 bending inwardly from the first reference plane S2201.

In another configuration, the second connector (top link) 2750 may have a second arm 2752, a fourth arm 2754 and an upper platform 2116. The upper platform 2116 may connect the second arm 2752 to the fourth arm 2754 to form a second arc along the second reference plane S2202. Viewing from the top and from the bottom, the first arc and the second arc may join at the pivot axis Ax to form the deflected X-shape bridge. The fourth arm 2754 may have a fourth arm extension 2756 bending inwardly from the second reference plane S2202. The third arm extension 2716 and the fourth arm extension 2756 allows the third arm 2714 and the fourth arm 2754 to extend the vertical reach without sacrificing much of their respective horizontal reach. This reach can allow a surgeon to work around the specific anatomy of a given patient.

The second arm 2752 may have a second arm extension 2755 deviating from the second reference plane S2202. The second arm extension 2755 may form a second (right) slanted V-shape strip protruding outwardly from the second reference plane S2202. Together, the first and second slanted V-shape strips allows the first arm 2712 and the second arm 2752 to extend the horizontal reach without substantially extending their respective vertical reach. Moreover, the first and second slanted V-shape strips may form a double-dipped valley for surrounding the base section of a spinous process. Although the second alternative embodiment shows that the deflected X-shape bridge has a double-dipped valley directly above the pivot joint 2130, the RXC cross connector 2700 may include other types of deflected X-shape bridges that may conform to the shape of a spinous process or used in cases of cervical and/or thoracalumbar laminectomy where a portion of the spinous process is taken out, thus removing protection provided by the spinous process.

In order to provide several anchoring points for the RXC cross connector 2700, each of the arm extensions may have a distal end for pivoting the rods. In one configuration, for example, the first arm extension 2715 may have a first distal end 2711, the second arm extension 2755 may have a second distal end 2751, the third arm extension 2716 may have a third distal end 2713, and a fourth arm extension 2756 may have a fourth distal end 2753. The rods may be inserted into the pedicle screw or system horizontally, vertically, or in any other configuration that allows the pedicle system to securely hold a portion of the rod when fastened. In an alternative configuration, one or more of the arm extensions (e.g., 2715, 2755, 2716, 2756) may have a longer length so as to mate with the pedicle system without the need for any connected rods (2101, 2102, 2103, 2104).

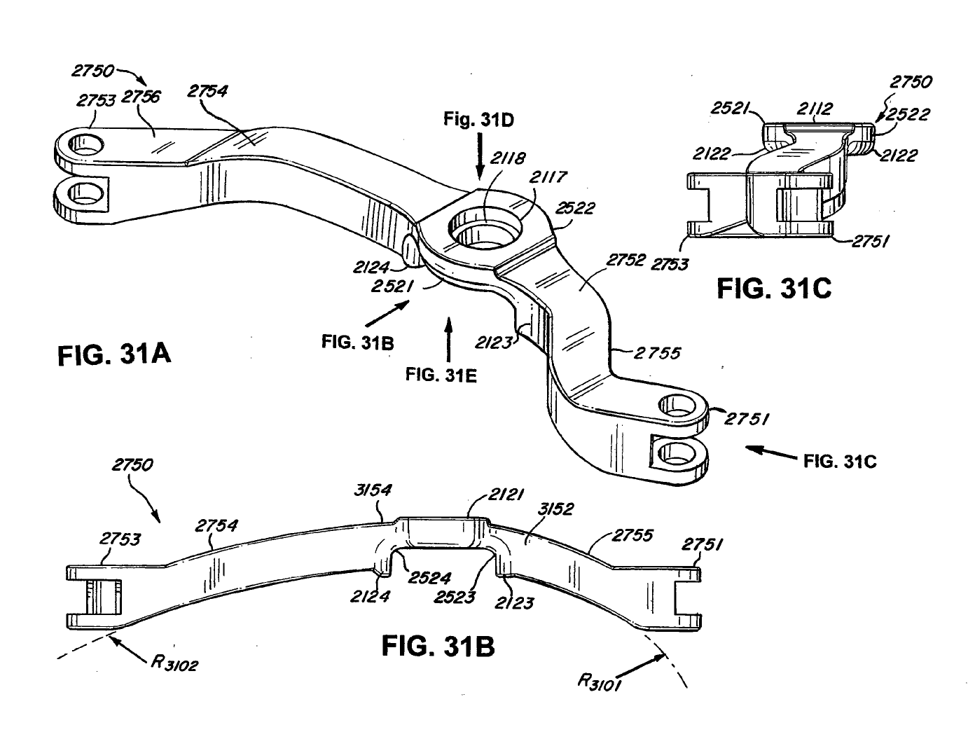

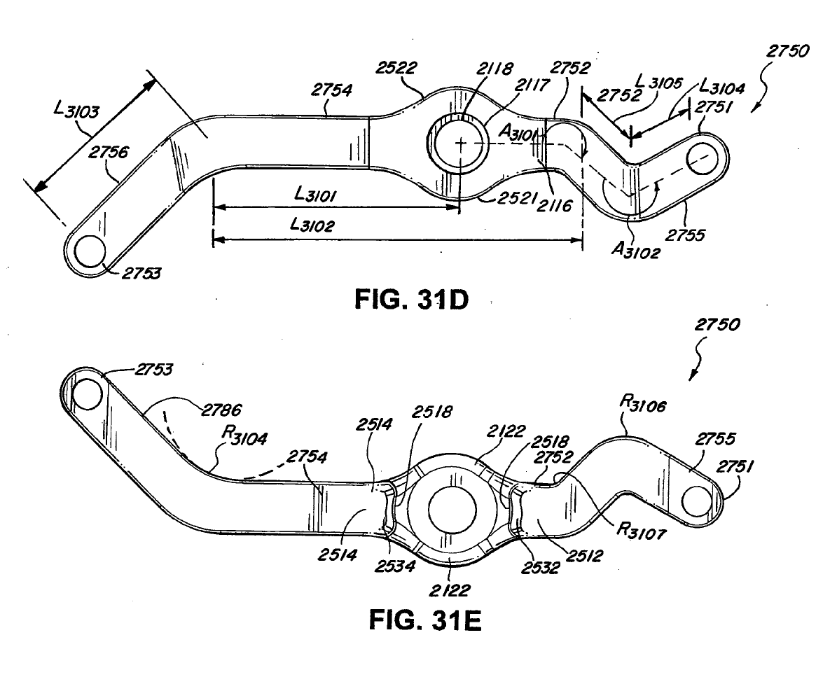

The discussion now turns to various dimensions of the first connector (bottom link) 2710 and the second connector (top link) 2750. Referring to FIG. 31D, the fourth arm 2754 may extend from the pivot axis by a first length L3101, the fourth arm 2754 may extend from the second arm 2752 by a second length L3102. In one configuration, the first length L3101 may be about 29.7 mm, and the second length L3102 may be about 42.9 mm. The V-shaped second arm extension 2755 may have a first segment and a second segment. The first segment may be adjacent to the second distal end 2751, and it may have a fourth length. The second segment may be adjacent to the second arm 2752, and it may have a fifth length L3105. In one configuration, the fourth length L3104 may be about 8.66, and the fifth length L3105 may be about 6.41.

A first angle A3101 may be formed between the second arm 2752 and the second segment of the second arm extension 2755, and a second angle A3102 may be formed between the first segment and the second segment of the second arm extension 2755. In one configuration, the first angle A3101 may be about 225 degrees, and the second angle A3102 may be about 255 degrees. In an alternative configuration, no bends or angles may be used.

Referring to FIG. 31B, a first curvature 83101 may be defined by the second arm 2752 and the second arm extension 2755, and a second curvature 83102 may be defined by the fourth arm 2754 and the fourth arm extension 2756. Generally, the first curvature 83101 may be steeper than the second curvature 83102. In one configuration, for example, the first curvature 83101 may have a radius of about 42.25 mm, while the second curvature 83102 may have a radius of about 107.59 mm.

Referring to FIG. 31E, the transition angles between an arm and an arm extension may be smoothened by a particular curvature. Such an angle-smoothening construction may help reduce the stress concentration around the transition angels, thereby enhancing the rigidity of the RXC cross connector 2700. A third curvature 83104 may smoothen the transition angle between the fourth arm 2754 and the fourth arm extension 2756. A fourth curvature R3107 may smoothen the first transition angle A3101, and a fifth curvature R3106 may smoothen the second transition angle A3102. In one configuration, the fourth curvature R3107, as well as the fifth curvature R3106, may each have a radius of about 6 mm.

The corresponding and/or matching parts of the second connector 2750 may have dimensions that are similar to those of the first connectors 2710. As such, the dimensions of the second connector 2750 are disclosed by reference to FIGS. 31B-31E. Moreover, the dimensions of several parts of the pivot joint 2130 are similar to those of the RXB cross connector 2100, such that these dimensions are disclosed by reference to FIGS. 25A-25E and 26A-26E.

The discussion now turns to several performance tests of the RXB cross connector 2100 and the RXC cross connector 2700. These performance tests were based on one or more computer aided design (CAD) models of the conventional cross connector (e.g., a horizontal connector connecting two segments of vertical rods), the RXB cross connector 2100, and the RXC cross connector 2700. Moreover, these performance tests were intended to compare the rigidity and stability of these cross connector under various ranges of bending load and torsion load. The CAD models of these cross connectors (i.e., the conventional cross connector, the RXB cross connector 2100, and the RXC cross connector 2700) were assembled to create virtual geometry consistent with the ASTM F1717 standard (a.k.a. “Standard Test Methods for Spinal Implant Constructs in a Vertebrectomy Model”). Finite element analysis (FEA) was performed on the virtual geometry using a validated modeling technique, including the material properties of these cross connectors (e.g., titanium) and the spinal bone segments (e.g., Ultra-high-molecular-weight polyethylene).

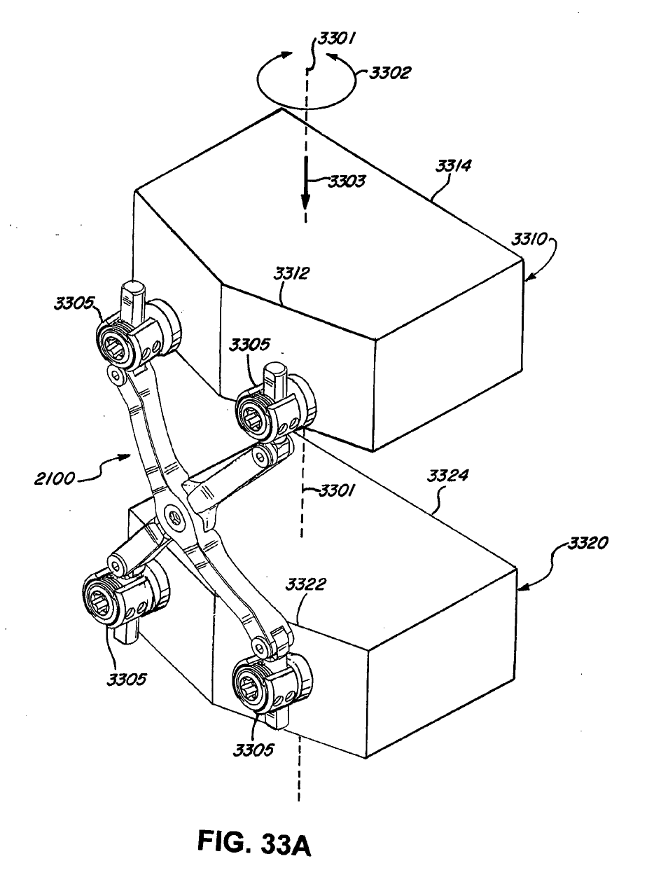

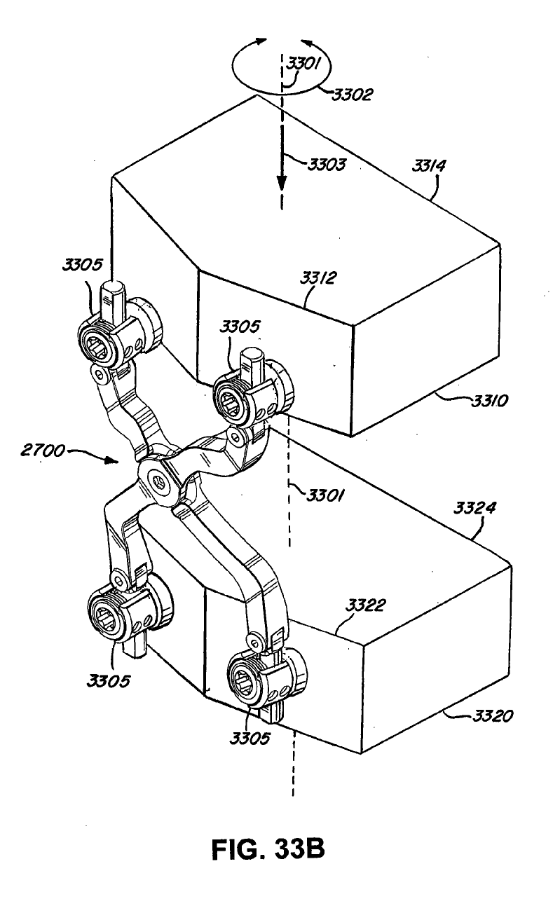

FIGS. 33A and 33B shows the perspective views of a stress test set up for the RXB cross connector 2100 the RXC cross connector 2700 respectively. The RXB cross connector 2100 and the RXC cross connector 2700 were separately and individually anchored to a first block 3310 and a second block 3320 by four pedicle screws 3305. More specifically, the first arm 2112 (or the first arm 2712) and the second arm 2152 (or the second arm 2752) were anchored to the back side 3312 of the first block 3310, while the third arm 2114 (the third arm 2714) and the fourth arm 2154 (or the fourth arm 2754) were anchored to the back side 3322 of the second block 3320. Each of the first block 3310 and the second block 3320 were used to simulate the property of one or more spinal bone segments. The back sides 3312 and 3322 represented the sides on which the spinous processes developed, while the front sides 3314 and 3324 represented the sides to which a patient might face.

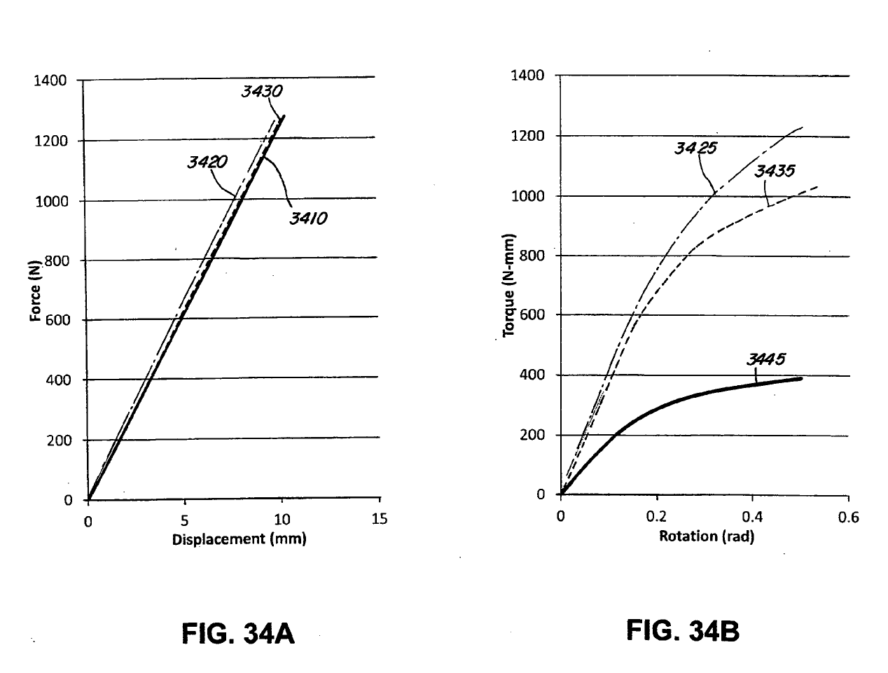

To conduct the linear displacement test, a bending load 3303 was applied to the first block 3310 along a reference axis 3301 while the second block 3320 was held at a constant position. The linear displacement test then measured the relative vertical displacement between the front side 3314 of the first block 3310 and the front side 3324 of the second block 3320. Referring to FIG. 34A, which shows a chart of the linear displacement test results, both the RXB cross connector result 3420 and the RXC cross connector result 3430 outperformed the conventional cross connector result 3410 over a wide range of bending load (measured in Newton “N”).

To conduct the angular displacement test, a torsion load 3302 was applied to the first block 3310 about the reference axis 3301 while the second block 3320 was held at a constant position. The angular displacement test then measured the relative angular displacement between the front surface 3314 of the first block 3310 and the front surface 3324 of the second block 3320. Referring to FIG. 34B, which shows a chart of the angular displacement test results, both the RXB cross connector result 3425 and the RXC cross connector result 3435 outperformed the conventional cross connector result 3445 over a wide range of torsion load (measured in Newton-millimeter “N-mm”).

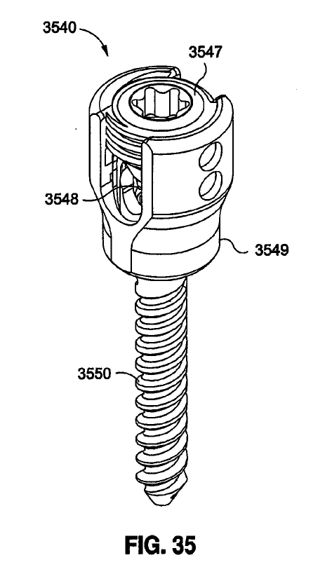

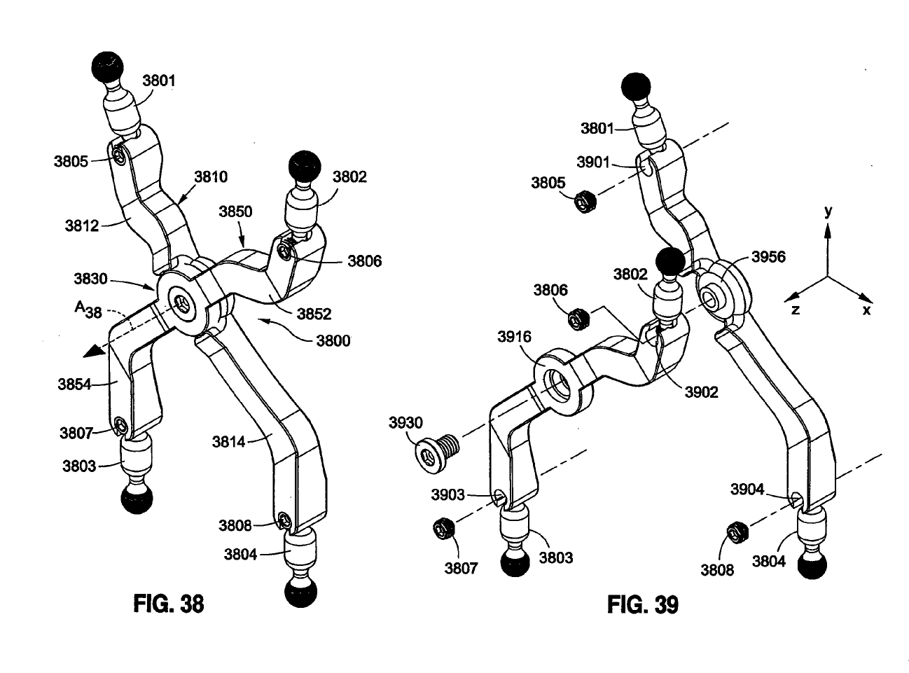

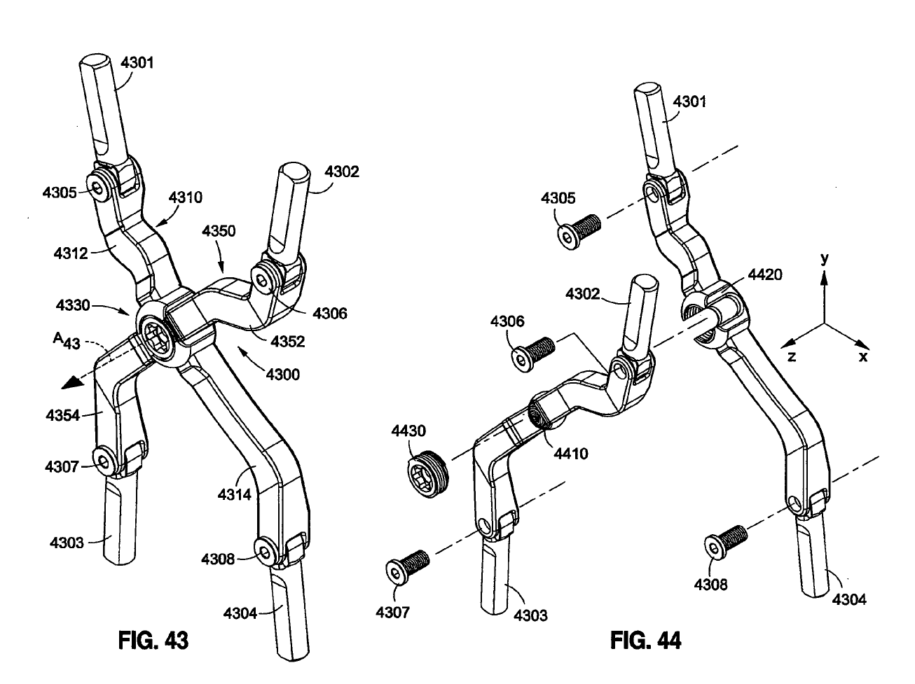

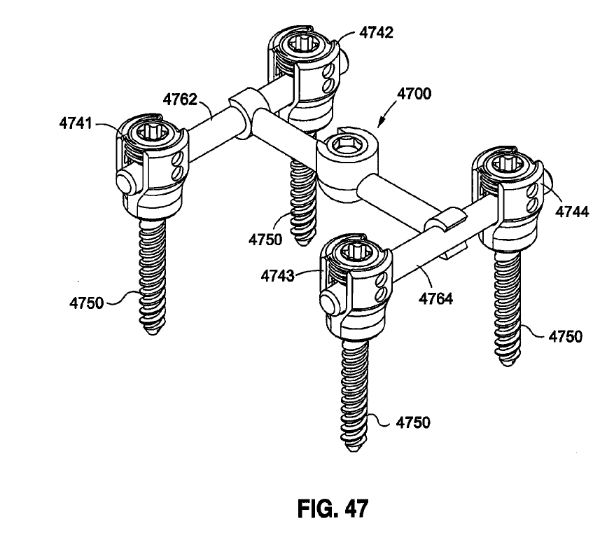

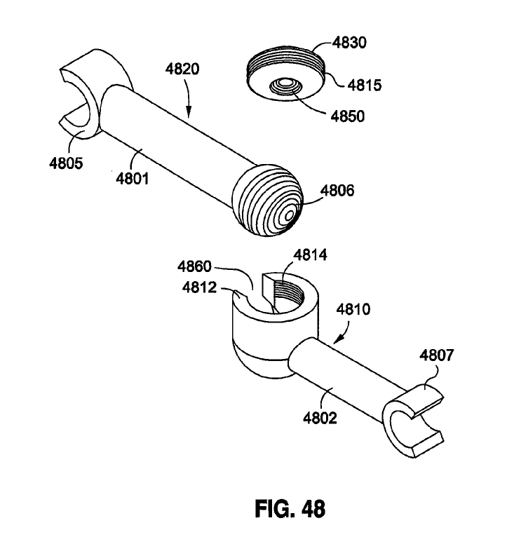

The discussion now turns to alternative embodiments of Real-X cross connectors or spinal bridges incorporating spherical joints. Spherical joints may provide a more adaptable apparatus that can accommodate any angle of any degenerative spine. By easily adjusting to the various spinal shapes, sizes, or configurations of different patients, spherical joints can provide easier and/or less time consuming surgical installations. A spherical joint may used in a pedicle screw, similar to those previously discussed for FIGS. 13A-20C for connection to a variety of connecting rods, the structural and functional features disclosed by FIGS. 35-37B. Spherical joints may be used as arm joints in alternative embodiments of Real-X cross connectors, the structural and functional features disclosed by FIGS. 38-42. Moreover, a spherical joint may be used as a fulcrum in an alternative embodiment of a Real-X cross connector, the structural and functional features disclosed by FIGS. 43-46B. In addition, a spherical joint may also be incorporated into a spinal bridge without a crossed configuration, the structural and functional features disclosed by FIGS. 47-48.

FIG. 35 shows a perspective view of a pedicle screw 3540 utilizing a spherical joint. Similar to the pedicle screws 1320, 1330, 1340, or 1350, and as discussed for FIGS. 13A-20C, the pedicle screw 3540 may be used to anchor a Real-X cross connector or other mechanical components to a spinal bone segment. Multiple pedicle screws 3540 may be used to anchor the Real-X cross connector or other mechanical components to a plurality of spinal bone segments. Generally, the pedicle screw 3540 includes a set screw 3547, a threaded shaft 3550, and a base member 3549. More specifically, the threaded shaft 3550 may be used for drilling into the spinal bone segment, the base member 3549 may have a pair of receiving ports 3548, and the set screw 3547 may be used for securing a portion of a Real-X cross connector or other mechanical component (such as a stabilizing rod) to the base member 3549.

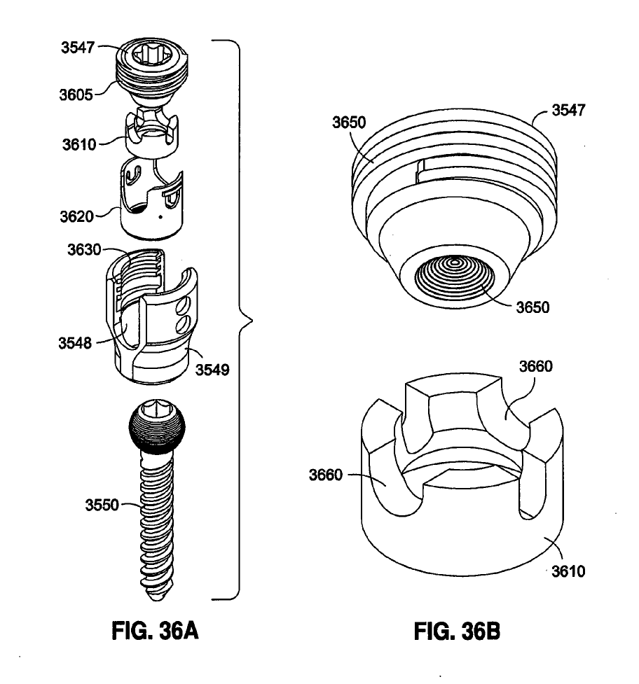

FIG. 36A shows a disassembled view of the pedicle screw 3540 to better illustrate its component parts. In addition to the set screw 3547, the threaded shaft 3550, and the base member 3549, a spherical compression saddle 3610 and an intermediate element 3620 fit within the base member 3549. The set screw 3547 includes a threaded portion 3605 disposed along an outer circumference of the set screw 3547. Similarly, the base member 3549 includes a threaded portion 3630 disposed along an inner circumference of the base member 3549. The threaded portion 3630 of the base member 3549 is adapted to engage with the threaded portion 3605 of the set screw 3547 in order to secure the set screw 3547 to the base member 3549. When assembled, the pedicle screw 3540 maintains the spherical compression saddle 3610 within the base member 3549 and beneath the set screw 3547. The set screw 3547 may be a cannulated screw.

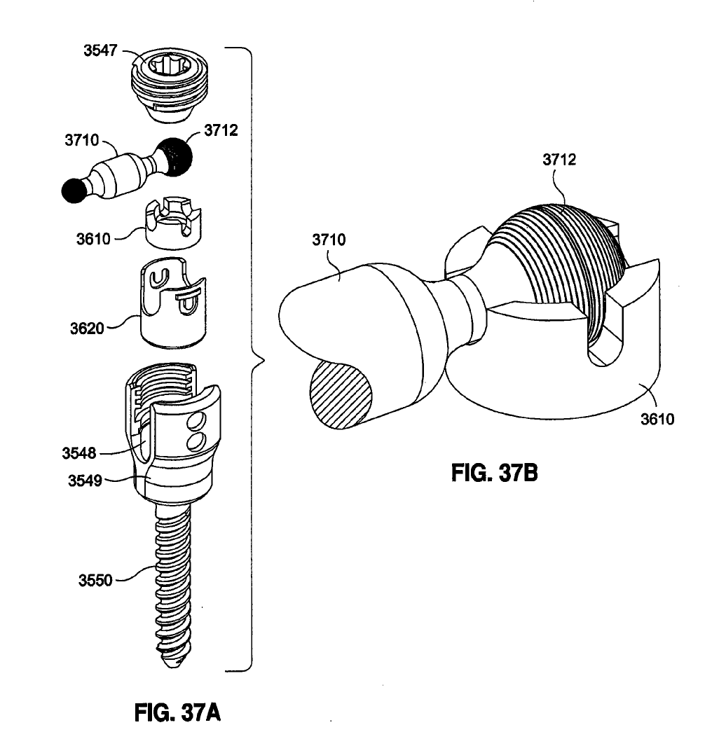

FIG. 36B is a zoomed-in view of the set screw 3547 and the spherical compression saddle 3610. The spherical compression saddle 3610 contains a hollow or open portion and one or more openings or ports 3660 disposed along the walls surrounding the hollow or open portion. The spherical compression saddle 3610 is configured to accept a substantially spherical element, as shown and discussed in greater detail for FIGS. 37A and 37B. The set screw 3547 includes a semi-spherical depression 3650 configured to engage with the substantially spherical element that is can be accepted and positioned in the spherical compression saddle 3610.

To better make frictional contact between the set screw 3547 and the substantially spherical element, the semi-spherical depression 3650 and/or the substantially spherical element may have a rough or uneven surface for improving the grip between the semi-spherical depression 3650 and the substantially spherical element when they are in contact with one another. The rough or uneven surface may be created by a plurality of protrusions and/or recessions. In one embodiment, the rough or uneven surface may be created via a plurality of concentric circles. Such concentric circles may be less prone to breaking, chipping or wearing down upon frictional contact with the substantially spherical element. In an alternative embodiment, a variety of other shapes or configurations may be used for creation of the rough or uneven surface. The rough or uneven surface may be formed by a variety of manufacturing processes, for example by brushing, sandblasting, milling and/or drilling.

FIG. 37A shows a disassembled view of the pedicle screw 3540 and also includes a connecting rod 3710 for engaging with the pedicle screw 3540. The connecting rod 3710 may be a discrete component piece or may be a continuation of an extension arm of a Real-X cross connector. The connecting rod 3710 is shown with a substantially spherical element 3712 disposed on both its distal and proximal end. An alternative embodiment may utilize only one substantially spherical element 3712. FIG. 37B shows a zoomed-in view of one of the substantially spherical elements 3712 of the connecting rod 3710 seated in the spherical compression saddle 3610. Before being secured with the set screw 3547, the connecting rod 3710 is free to rotate in three dimensions via the substantially spherical element 3712 seated in the spherical compression saddle 3610. This range of rotation is limited by one of the ports 3660 of the spherical compression saddle 3610, as shown in FIG. 36B.

The substantially spherical element 3712 has a rough or uneven surface for improved grip with the semi-spherical depression 3650 of the set screw 3547 when the substantially spherical element 3712 is engaged with the semi-spherical depression 3650. Improving the frictional contact between the two components helps maintain the connecting rod 3710 in the desired position after installation is complete and helps prevent slippage that might otherwise occur between the substantially spherical element 3712 and the semi-spherical depression 3650. As discussed for FIG. 36B, the rough or uneven surface may utilize a plurality of concentric circles as shown, or may utilize other shapes or configurations.

FIG. 38 shows a perspective view of a Real-X cross connector 3800 utilizing spherical joints according to one embodiment of the present invention. The Real-X cross connector 3800 may be used for stabilizing and protecting one or more fixation levels of spinal bone segments while providing an easily adjustable means of attachment to a patient's body. The Real-X cross connector 3800 may be similar to the cross connectors 2100 or 2700 previously discussed for FIGS. 21-32E. As such, one skilled in the art may readily understand and appreciate these similar features by referencing the previous discussion and thus the detailed description of certain previously described features will not be repeated or will not be repeated in full detail in the following sections. The Real-X cross connector 3800 may be adjustably equipped with several connecting rod segments having spherical joints, such as a first rod 3801, a second rod 3802, a third rod 3803, and a fourth rod 3804. Each of the first rod 3801, the second rod 3802, the third rod 3803, and the fourth rod 3804 may be the same or similar to the double spherical rod 3710, discussed above for FIGS. 37A and 37B. The Real-X cross connector 3800 may be affixed to a plurality of spinal bone segments by anchoring the connecting rod segments (e.g., the first rod 3801, the second rod 3802, the third rod 3803, and/or the fourth rod 3804) to the pedicle areas of these spinal bone segments. For example, one or more pedicle screws 3540, discussed above for FIGS. 35-37B, may be used as anchoring devices for anchoring the connecting rod segments to the pedicle areas of the spinal bone segments.

The Real-X cross connector 3800 may include a first connector (bottom link) 3810, a second connector (top link) 3850, and a pivot joint 3830. In order to form an X-shaped or a deflected X-shaped bridge across the targeted spinal bone segments, the pivot joint 3830 may pivot the mid section of the first connector 3810 against the mid section of the second connector 3850. In one implementation, for example, the pivot joint 3830 may be an integral part of the first connector 3810 and the second connector 3850. In another implementation, for example, the pivot joint 3830 may be a separate part of the first connector 3810 and/or the second connector 3850. In yet another implementation, for example, the pivot joint 3830 may be partially integrated with the first connector 3810 and/or the second connector 3850.

The first connector 3810 of the Real-X cross connector 3800 includes a first arm 3812 and a third arm 3814. Similarly, the second connector 3850 of the Real-X cross connector 3800 includes a second arm 3852 and a fourth arm 3854. As discussed herein, the numerical terms, such as “first,” “second,” “third,” and “fourth” are relative terms such that they may be used interchangeably. Moreover, as discussed herein, the positioning terms, such as “top” and “bottom” are relative terms such that they may also be used interchangeably.

The first arm 3812 may be spherically connected to the first rod 3801 via a first screw 3805. When the first screw 3805 is not fastened, the first rod 3801 may have a range of spherical movement about the end of the first arm 3812 or the first screw 3805. When the first screw 3805 is substantially fastened, the first rod 3801 may be tightly connected to the first arm 3812 such that the relative motion between the first rod 3801 and the first arm 3812 may be substantially restricted.

The third arm 3814 may be spherically connected to the fourth rod 3804 via a fourth screw 3808. When the fourth screw 3808 is not fastened, the fourth rod 3804 may have a range of spherical movement about end of the third arm 3814 or the fourth screw 3808. When the fourth screw 3808 is substantially fastened, the fourth rod 3804 may be tightly connected to the third arm 3814 such that the relative motion between the fourth rod 3804 and the third arm 3814 may be substantially restricted.

The second arm 3852 may be spherically connected to the second rod 3802 via a second screw 3806. When the second screw 3806 is not fastened, the second rod 3802 may have a range of spherical movement about end of the second arm 3852 or the second screw 3806. When the second screw 3806 is substantially fastened, the second rod 3802 may be tightly connected to the second arm 3852 such that the relative motion between the second rod 3802 and the second arm 3852 may be substantially restricted.

The fourth arm 3854 may be spherically connected to the third rod 3803 via a third screw 3807. When the third screw 3807 is not fastened, the third rod 3803 may have a range of spherical movement about the end of the fourth arm 3854 or the third screw 3807. When the third screw 3807 is substantially fastened, the third rod 3803 may be tightly connected to the fourth arm 3854 such that the relative motion between the third rod 3803 and the fourth arm 3854 may be substantially restricted.

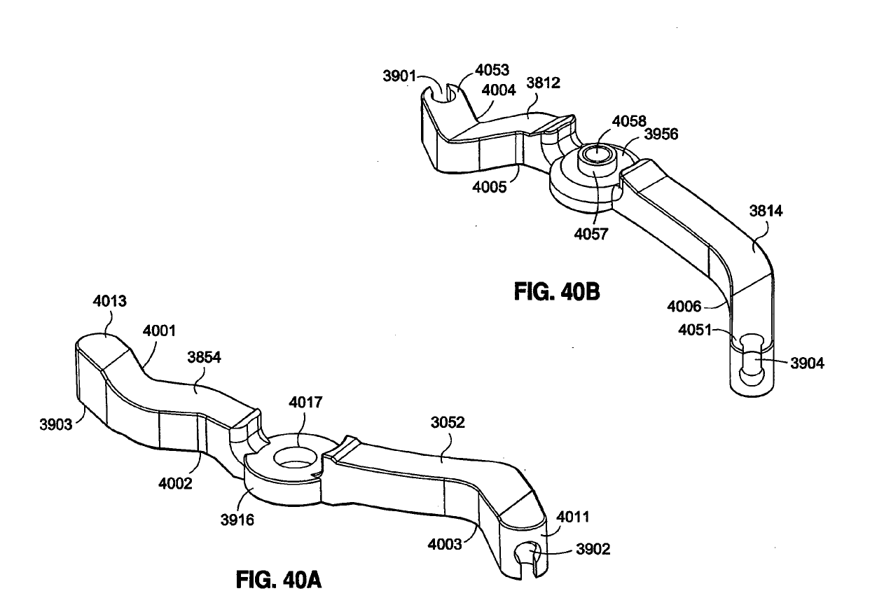

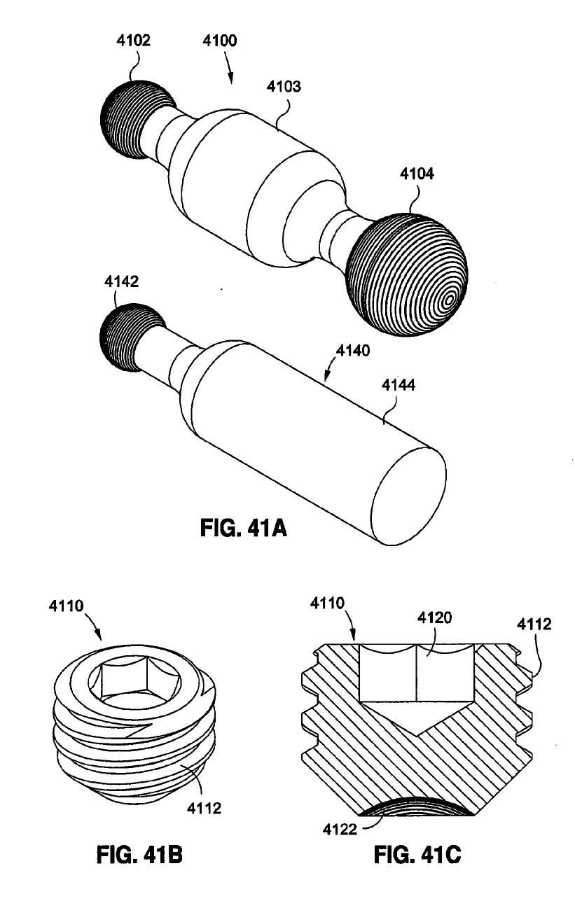

Turning now to FIG. 39, with reference to FIG. 38, a disassembled view of the Real-X cross connector 3800 is shown. The first connector 3810 (a lower transverse arm) includes a lower platform 3956. The second connector 3850 (an upper transverse arm) includes an upper platform 3916. The upper platform 3916 may connect the first arm 3812 to the third arm 3814, such that the first arm 3812 and the third arm 3814 may form a contiguous arc segment making up the first connector 3810. The first connector 3810 may be disposed along a first reference plane or may incorporate curves or other structural configurations as discussed in greater detail for FIGS. 40A and 40B. Similarly, the lower platform 3856 may connect the second arm 3852 to the fourth arm 3854, such that the second arm 3852 and the fourth arm 3854 may form another contiguous arc segment making up the second connector 3850. The second connector 3850 may be disposed along a second reference plane or may incorporate curves or other structural configurations as discussed in greater detail for FIGS. 40A and 40B. When mated together, the first connector 3810 and the second connector 3850 may appear as two elongated connector members crossing each other so as to form a substantially X-shaped or deflected X-shaped protection bridge. The first connector 3810 and/or second connector 3850 may be configured to accept one or more rods as discussed in greater detail below, or, in an alternative embodiment, may include as part of the first connector 3810 and/or second connector 3850, one or more spherical ends.

A first opening 3901 in the first arm 3812 of the first connector 3810 is configured to receive a portion of the first rod 3801. When received by the first opening 3901, the first rod 3801 is permitted to rotate about the first arm 3812 in three dimensions before being secured by the first screw 3805. The size and/or shape of the first opening 3901 will limit the degree of rotation that may be exhibited by the first rod 3801 before the first screw 3805 securely fastens the first rod 3801 to the first arm 3812.

A second opening 3902 in the second arm 3852 of the second connector 3850 is configured to receive a portion of the second rod 3802. When received by the second opening 3902, the second rod 3802 is permitted to rotate about the second arm 3852 in three dimensions before being secured by the second screw 3806. The size and/or shape of the second opening 3902 will limit the degree of rotation that may be exhibited by the second rod 3802 before the second screw 3806 securely fastens the second rod 3802 to the second arm 3852.

A third opening 3903 in the fourth arm 3854 of the second connector 3850 is configured to receive a portion of the third rod 3803. When received by the third opening 3903, the third rod 3803 is permitted to rotate about the fourth arm 3854 in three dimensions before being secured by the third screw 3807. The size and/or shape of the third opening 3903 will limit the degree of rotation that may be exhibited by the third rod 3803 before the third screw 3807 securely fastens the third rod 3803 to the fourth arm 3854.

A fourth opening 3904 in the third arm 3814 of the first connector 3810 is configured to receive a portion of the fourth rod 3804. When received by the fourth opening 3904, the fourth rod 3804 is permitted to rotate about the third arm 3814 in three dimensions before being secured by the fourth screw 3808. The size and/or shape of the fourth opening 3904 will limit the degree of rotation that may be exhibited by the fourth rod 3804 before the fourth screw 3808 securely fastens the fourth rod 3804 to the third arm 3814.

FIG. 40A shows a zoomed-in view of the second connector 3850 (an underside view of the upper transverse arm) and FIG. 40B shows a zoomed-in view of the first connector 3810 (a topside view of the lower transverse arm). The distance between the openings at each end of the first and second connectors 3810 and 3850 (e.g., the first opening 3901, the second opening 3902, the third opening 3903, and/or the fourth opening 3904) may define the reach of the Real-X cross connector 3800. The first connector 3810 and/or the second connector 3850 may also contain a number of curves or bends along their respective lengths to form a deflected X-shape bridge and providing the benefit of better fitting around the spinous process of the spinal bone segments. More specifically, first curve 4001, second curve 4002, third curve 4003, fourth curve 4004, fifth curve 4005, and sixth curve 4006 along the first connector 3810 and the second connector 3850 are included to provide clearance around a patient's spinous process that might otherwise need to be removed for fitment of a bridge across the spinal bone segments. Moreover, the first connector 3810 and/or the second connector 3850 may also incorporate an arced configuration so as to extend the Real-X cross connector outwardly along the axis A₃₈ and away from the spinal bone segments when the Real-X cross connector 3800 is installed in a patient. Such a configuration can provide an additional protective or safety benefit against impacts to the spinal bone segments from outside the body of the patient.

With reference to FIG. 38-39, the upper platform 3916 of the second connector 3850 may interpose the lower platform 3956 of the first connector 3810 along and about a center axis. The lower platform 3956 may include one or more components for engaging the upper platform 3916. Such an engagement may provide a pivoting point for the Real-X cross connector 3800, thereby allowing the Real-X cross connector 3800 to be adjustable in order to fit varying spinal proportions of different patients. For example, pivoting the first connector 3810 with respect to the second connector 3850 at the engagement of the lower platform 3956 to the upper platform 3916 can adjustably lengthen or shorten the distance between the ends of the first arm 3812 and the fourth arm 3854 or the ends of the second arm 3852 and the third arm 3814. Similarly, pivoting the first connector 3810 with respect to the second connector 3850 at the engagement of the lower platform 3956 to the upper platform 3916 can adjustably lengthen or shorten the distance between the ends of the first arm 3812 and the second arm 3852 or the ends of the third arm 3814 and the fourth arm 3854.

Moreover, the upper platform 3916 may establish a complementary relationship with the lower platform 3956. In one configuration, the upper platform 3916 may include an opening 4017 and the lower platform 3956 may include a hollow protrusion or pole 4057. The opening 4017 of the upper platform is configured to receive the hollow protrusion or pole 4057 of the lower platform 3956 such that when the upper platform 3916 and the lower platform 3956 are complementary configured and positioned, the first connector 3810 is snugly fitted with the second connector 3850 at the pivot joint 3830. A center screw 3930 with a threaded shaft may fit within the opening 4017 of the upper platform 3916 and within the hollow protrusion or pole 4057. A threaded interior surface 4058 of the hollow protrusion or pole 4057 engages with the threaded shaft of the center screw 3930 to secure the center screw 3930, the upper platform 3916 and the lower platform 3956 together.

When the set screw 3930 partially engages the threaded interior surface 4058 of the hollow pole 4057, the first connector 3810 may freely rotate about the pivot joint while the upper platform 3916 remains substantially in contact with the lower platform 3956. When the set screw 3930 substantially engages the threaded interior surface 4058, the lower platform 3956 is forced against the upper platform 3916. As a result, a pair of action and reaction forces may be asserted against the inner surfaces of the upper platform 3916 and the lower platform 3956. The action and reaction forces may substantially restrict the relative rotational movement between the upper platform 3916 and the lower platform 3956, thereby locking the Real-X cross connector 3800 into a particular angle at the pivot joint 3830. Other aspects of the pivoting means may be as described above in previous embodiments.

In addition to the pivot joint 3830 created substantially at the center of the Real-X cross connector 3800 by the connection between the upper platform 3916 and lower platform 3956, four additional joint locations are disposed along the structural body of the Real-X cross connector 3800. Rods connected at the additional joint locations may provide the anchoring means for fastening the Real-X cross connector 3800 to the spinal segments of a patient. As previously discussed for FIG. 39, the first opening 3901 in the first arm 3812 of the first connector 3810 is configured to receive a portion of the first rod 3801. A second opening 3902 in the second arm 3852 of the second connector 3850 is configured to receive a portion of the second rod 3802. A third opening 3903 in the fourth arm 3854 of the second connector 3850 is configured to receive a portion of the third rod 3803. A fourth opening 3904 in the third arm 3814 of the first connector 3810 is configured to receive a portion of the fourth rod 3804.

FIG. 41A shows a double spherical rod 4100 and a single spherical rod 4140, each of which may be the same or similar to each of the first rod 3801, the second rod 3802, the third rod 3803 or the fourth rod 3804. The double spherical rod 4100 has a first spherical end 4102 and a second spherical end 4104 connected by a middle portion 4103. The first spherical end 4102 may be smaller in diameter than the second spherical end 4104 (e.g. roughly 3 mm in diameter versus roughly 5 mm in diameter) or, in an alternative embodiment, the first spherical end 4102 may be the same size or greater in diameter than the second spherical end 4014. The first spherical end 4102 and/or the second spherical end 4104 may be formed with a rough or uneven surface, such as protruding or recessing concentric circles, for better making frictional contact with connecting components, as described in greater detail for FIG. 41C. The single spherical rod 4140 has a spherical end 4142 and a non-spherical end 4144 which may be cylindrical in shape. In one embodiment, the spherical end 4142 may be roughly 3 mm in diameter and/or the non-spherical end 4144 may be roughly 13 mm in length. The spherical end and/or the non-spherical end may be formed with a rough or uneven surface, similar to that of the double spherical rod 4100.

When used as the first rod 3801, the double spherical rod 4100 has the first spherical end 4102 sized and/or shaped to fit within the first opening 3901 of the first arm 3812. When used as the second rod 3802, the double spherical rod 4100 has the first spherical end 4102 sized and/or shaped so to fit within the second opening 3902 of the second arm 3852. When used as the third rod 3803, the double spherical rod 4100 has the first spherical end 4102 sized and/or shaped so to fit within the third opening 3903 of the fourth arm 3854. When used as the fourth rod 3804, the double spherical rod 4100 has the first spherical end 4102 sized and/or shaped so to fit within the fourth opening 3904 of the third arm 3814.

The first additional joint location of the Real-X cross connector 3800, for example, may be created at the first opening 3901. When the first screw 3805 has not securely engaged the first rod 3801 with the first arm 3812, the first rod 3801 may freely rotate in three dimensions about the end of the first arm 3812, limited by the size and/or shape of the first opening 3901. When the first screw 3805 substantially engages the first rod 3801 with the first arm 3812, the rotational movement of the first rod 3801 is substantially restricted. As such, the first rod 3801 can be locked in a particular position with respect to the end of the first arm 3812.

The second additional joint location of the Real-X cross connector 3800, for example, may be created at the second opening 3902. When the second screw 3806 has not securely engaged the second rod 3802 with the second arm 3852, the second rod 3802 may freely rotate in three dimensions about the end of the second arm 3852, limited by the size and/or shape of the second opening 3902. When the second screw 3806 substantially engages the second rod 3802 with the second arm 3852, the rotational movement of the second rod 3802 is substantially restricted. As such, the second rod 3802 can be locked in a particular position with respect to the end of the second arm 3852.

The third additional joint location of the Real-X cross connector 3800, for example, may be created at the third opening 3903. When the third screw 3807 has not securely engaged the third rod 3803 with the fourth arm 3854, the third rod 3803 may freely rotate in three dimensions about the end of the fourth arm 3854, limited by the size and/or shape of the third opening 3903. When the third screw 3807 substantially engages the third rod 3803 with the fourth arm 3854, the rotational movement of the third rod 3803 is substantially restricted. As such, the third rod 3803 can be locked in a particular position with respect to the end of the fourth arm 3854.

The fourth additional joint location of the Real-X cross connector 3800, for example, may be created at the fourth opening 3904. When the fourth screw 3808 has not securely engaged the fourth rod 3804 with the third arm 3814, the fourth rod 3804 may freely rotate in three dimensions about the end of the third arm 3814, limited by the size and/or shape of the fourth opening 3904. When the fourth screw 3808 substantially engages the fourth rod 3804 with the third arm 3814, the rotational movement of the fourth rod 3804 is substantially restricted. As such, the fourth rod 3804 can be locked in a particular position with respect to the end of the third arm 3814.

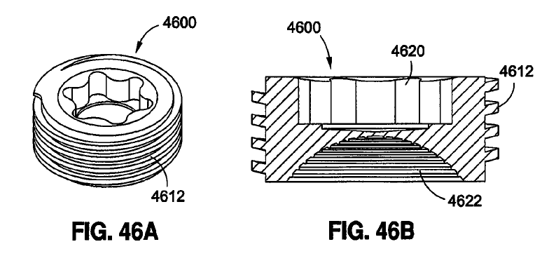

With reference to FIGS. 38-40B, FIG. 41B shows a set screw 4110 that may be the same or similar to any of the first screw 3805, the second screw 3806, the third screw 3807, or the fourth screw 3808. The set screw 4110 may be cannulated or non-cannulated. Furthermore, certain features of the locking screw 1201, discussed for FIG. 12A-12D, and/or the set screw 4600, discussed for FIG. 46A-46B may be the same or similar to features of the set screw 4110. For example, the set screw 4110 may be configured to have a shallower profile and/or utilize a deeper or larger semi-spherical depression as shown for the set screw 4600, discussed in greater detail below. Upon rotating either the first rod 3801, the second rod 3802, the third rod 3803, or the fourth rod 3804 into a desired or particular position with respect to their respective ends of the Real-X cross connector 3800, each rod is secured in that position to prevent their movement after the installation in the patient is complete. The set screw 4110 includes a threaded portion 4112 disposed along an outer circumference for engaging the set screw 4100 with a connecting surface configured to receive such threading. For example, first screw 3805, which may be set screw 4110, can engage the threaded portion 4112 with an inner surface or lip that at least partially defines the first opening 3901 in order to secure the first screw 3805 to first arm 3812.

FIG. 41C shows a cross-section of the set screw 4110 to better illustrate its structural and functional features. A hollow portion 4120 at one end of the set screw 4110 provides an opening for the insertion of a screw driver or other mechanical component to facilitate the rotation of the screw into place via the engaging of the threaded portion 4112 with a receiving surface of one of the openings in the first or second connectors 3810 or 3850 (e.g., the first opening 3901, the second opening 3902, the third opening 3903, or the fourth opening 3904). A semi-spherical depression 4122 is disposed along a lower portion of the set screw 4110 and is configured to engage with a substantially spherical ball of a connecting rod or component. The semi-spherical depression may have a rough or uneven surface for better making frictional contact with the substantially spherical ball when the set screw 4110 is securely engaged with the substantially spherical ball. In one embodiment, the rough or uneven surface may be formed by a plurality of protruding or recessing concentric circles. Such concentric circles may maintain their uneven surface for longer periods due to the surface being more resistant to chipping or breaking when compared to smaller, non-contiguous protrusions making up the uneven surface.

In one example, the first rod 3801 may be the double spherical rod 4100 and the first screw 3805 may be the set screw 4110. When the set screw 4110 is not securely engaged with the first rod 3801, the first rod 3801 has minimal if any frictional contact with the semi-spherical depression of the first screw 3805 and is thus allowed to rotate in three dimensions about the first opening 3901 as previously discussed to a desired position. Upon securely engaging the first screw 3805 with the first rod 3801, the semi-spherical depression 4122 of the first screw 3805 accepts the a portion of the spherical end of the first rod 3801 and makes frictional contact with the portion of the spherical end of the first rod 3801 via the rough or uneven surface present on the semi-spherical depression 4122 and/or the spherical end of the first rod 3801. This frictional contact helps maintain the first rod 3801 in the desired position. The above description applies equally to the second rod 3802 with the second screw 3806, the third rod 3803 with the third screw 3807, and the fourth rod 3804 with the fourth screw 3808.

The double spherical rod 4100 or the spherical rod 4140 may have a rigid or a flexible construction. In a rigid embodiment, the double spherical rod 4100 or the spherical rod 4140 are manufactured such that the body portion between the ends of the rods does not flex or bend. In a flexible embodiment, for example, the double spherical rod 4100 or the spherical rod 4140 may be manufactured such that at least a portion of the rod forms a spring-like orientation. The spring may be tightly wound so the rod is substantially rigid, but capable of slight flexing when pressure is applied to one or both of the ends of the rod. Slight flexing of the rods 4100 or 4140 may provide for even greater adaptability during installation to a specific spinal proportion of a given patient. In addition, the rods 4100 or 4140 can be formed with various sizes and/or dimensions so as accommodate the spinous process of various patients. The double spherical rod 4100 or the spherical rod 4140 may be manufactured of stainless steel, titanium, PEEK, or any other alloy. Similarly, the double spherical rod 4100 or the spherical rod 4140 may be coated or plated with a variety of the same or other materials.

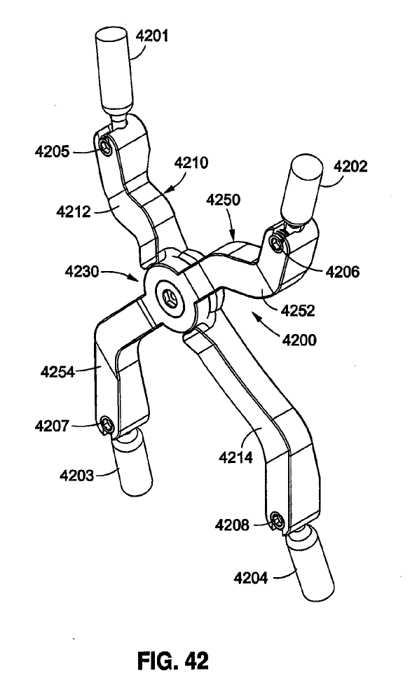

An alternative embodiment of a Real-X cross connector 4200 utilizing connecting rods with only a single spherical end is shown in perspective view in FIG. 42. Generally, the Real-X cross connector 4200 may have certain structure and functional features that are similar to those of the Real-X cross connector 3800, but is shown utilizing connecting rods 4201, 4202, 4203, and 4204 without dual spherical ends. The connecting rods 4201, 4202, 4203, and 4204 may be the spherical rod 4140 shown in FIG. 41A. The Real-X cross connector 4200 has a first connector 4210 having a first arm 4212 and a third arm 4214. The first connector 4210 may be the same or similar to the first connector 3810 of the Real-X cross connector 3800. Similarly, the Real-X cross connector 4200 has a second connector 4250 having a second arm 4252 and a fourth arm 4254. Likewise, the second connector 4250 may be the same or similar to the second connector 2850 of the Real-X cross connector 3800. A plurality of set screws 4205, 4206, 4207, and 4208 are used to fasten the connecting rods 4201, 4202, 4203, and 4204 to the first connector 4210 or second connector 4250 in the same or similar fashion as described above for the set screws 3805, 3806, 3807, and 3808. The Real-X cross connector 4200 mates the first connector 4210 with the second connector 4250 at a pivot joint 4230, the same or similar to the pivot joint 3830 of the Real-X cross connector 3800.

Turning next to FIG. 43, a perspective view of a Real-X cross connector 4300 is shown. Generally, the Real-X cross connector 4300 may have certain structure and functional features that are similar to those of the Real-X cross connector 3800 or Real-X cross connector 4200. Notwithstanding these similar features, the Real-X cross connector 4300 may be distinguished from the Real-X cross connector 3800 based primarily on the structure of a spherical center joint.

The Real-X cross connector 4300 may be adjustably equipped with several connecting rod segments, such as a first rod 4301, a second rod 4302, a third rod 4303, and a fourth rod 4304. Each of the first rod 4301, the second rod 4302, the third rod 4303, and the fourth rod 4304 may be the same or similar to the connecting rods 2101, 2102, 2103, or 2104, discussed above for FIGS. 21-24. In an alternative embodiment, each of the first rod 4301, the second rod 4304, the third rod 4303, and the fourth rod 4304 may be the same or similar to the connecting rods 3801, 3802, 3803, and 3804 or 4201, 4202, 4203, and 4204. The Real-X cross connector 4300 may be affixed to two or more spinal bone segments by anchoring the connecting rod segments (e.g., the first rod 4301, the second rod 4302, the third rod 4303, and/or the fourth rod 4304) to the pedicle areas of these spinal bone segments as previously discussed.

The Real-X cross connector 4300 may include a first connector (bottom link) 4310, a second connector (top link) 4350, and a spherical joint 4330. In order to form an adjustable X-shaped or deflected X-shaped bridge across the targeted spinal bone segments, the spherical joint 4330 permits rotation at the mid section of the first connector 4310 in three dimensions relative to the second connector 4350. In one implementation, for example, the spherical joint 4330 may be an integral part of the first connector 4310 and the second connector 4350. In another implementation, for example, the spherical joint 4330 may be a separate part of the first connector 4310 and/or the second connector 4350. In yet another implementation, for example, the spherical joint 4330 may be partially integrated with the first connector 4310 and/or the second connector 4350.

The first connector 4310 of the Real-X cross connector 4300 includes a first arm 4312 and a third arm 4314. Similarly, the second connector 4350 of the Real-X cross connector 4300 includes a second arm 4352 and a fourth arm 4354. As discussed herein, the numerical terms, such as “first,” “second,” “third,” and “fourth” are relative terms such that they may be used interchangeably. Moreover, as discussed herein, the positioning terms, such as “top” and “bottom” are relative terms such that they may also be used interchangeably.

The first arm 4312 may be pivotally connected to the first rod 4301 via a first screw 4305. When the first screw 4305 is not fastened, the first rod 4301 may have a range of pivotal movement about the end of the first arm 4312 or the first screw 4305. When the first screw 4305 is substantially fastened, the first rod 4301 may be tightly connected to the first arm 4312 such that the relative motion between the first rod 4301 and the first arm 4312 may be substantially restricted.

The third arm 4314 may be pivotally connected to the fourth rod 4304 via a fourth screw 4308. When the fourth screw 4308 is not fastened, the fourth rod 4304 may have a range of pivotal movement about end of the third arm 4314 or the fourth screw 4308. When the fourth screw 4308 is substantially fastened, the fourth rod 4304 may be tightly connected to the third arm 4314 such that the relative motion between the fourth rod 4304 and the third arm 4314 may be substantially restricted.

The second arm 4352 may be pivotally connected to the second rod 4302 via a second screw 4306. When the second screw 4306 is not fastened, the second rod 4302 may have a range of pivotal movement about end of the second arm 4352 or the second screw 4306. When the second screw 4306 is substantially fastened, the second rod 4302 may be tightly connected to the second arm 4352 such that the relative motion between the second rod 4302 and the second arm 4352 may be substantially restricted.

The fourth arm 4354 may be pivotally connected to the third rod 4303 via a third screw 4307. When the third screw 4307 is not fastened, the third rod 4303 may have a range of pivotal movement about the end of the fourth arm 4354 or the third screw 4307. When the third screw 4307 is substantially fastened, the third rod 4303 may be tightly connected to the fourth arm 4354 such that the relative motion between the third rod 4303 and the fourth arm 4354 may be substantially restricted.

Although non-spherical rods are shown in FIG. 43, it is envisioned that an alternative embodiment may employ any other type of connecting rod segments as the first rod 4301, the second rod 4302, the third rod 4303 or the fourth rod 4304. For example, the double spherical rod 4100 or the single spherical rod 4140 and associated fixation hardware may be used to connect to the Real-X cross connector 4300. Such a configuration would allow for three dimensional rotation at not only the center spherical joint 4330, but also at the ends of one or more of the first arm 4312, the second arm 4352, the third arm 4314, or the fourth arm 4354. An embodiment of this configuration may provide even greater installation flexibility in the body of a patient.

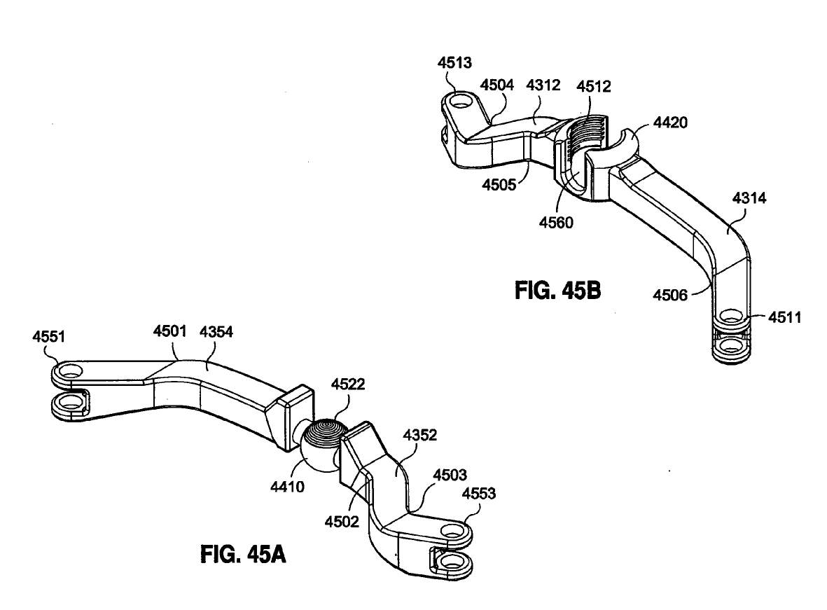

Turning now to FIG. 44, with reference to FIG. 43, a disassembled view of the Real-X cross connector 4300 is shown. The first connector 4310 includes a spherical housing 4420. The second connector 4352 includes a sphere 4410. A cannulated or non-cannulated set screw 4430 may be used to engage with the spherical housing 4420 and receive a portion of the sphere 4410, as described in greater detail for FIGS. 46A-B. The spherical housing 4420 may connect the first arm 4312 to the third arm 4314, such that the first arm 4312 and the third arm 4314 may form a contiguous arc segment making up the first connector 4310. The first connector 4310 may be disposed along a first reference plane or may incorporate curves or other structural configurations as discussed in greater detail for FIGS. 45A and 45B. Similarly, the center sphere 4410 may connect the second arm 4352 to the fourth arm 4354, such that the second arm 4352 and the fourth arm 4354 may form another contiguous arc segment making up the second connector 4350. The second connector 4350 may be disposed along a second reference plane or may incorporate curves or other structural configurations as discussed in greater detail for FIGS. 45A and 45B. When mated together, the first connector 4310 and the second connector 4350 may appear as two elongated connector members crossing each other so as to form a substantially X-shaped or deflected X-shaped protection bridge. At the end of each arm a connecting rod (e.g. 4301, 4302, 4303, 4304) may be fastened with screws 4305, 4306, 4307 or 4308 to enable connection to a pedicle screw or other spinal bone segment attachment mechanism as previously discussed. Each connecting rod may be attached with a pivotal joint as shown and as described in greater detail for FIGS. 21-24 or may be attached with a spherical joint as described in greater detail for FIGS. 38-41C. In an alternative embodiment, other connecting rods may be attached without any pivoting or rotating capabilities.

FIG. 45A shows a zoomed-in view of the second connector 4350 and FIG. 45B shows a zoomed-in view of the first connector 4310. The distance between the proximal end 4511 and the distal end 4513 of the first connector 4310 may define a first reach of the Real-X cross connector 4300. Similarly, the distance between the proximal end 4553 and the distal end 4551 of the second connector 4350 may define a second reach of the Real-X cross connector 4300. The first connector 4310 and/or the second connector 4350 may also contain a number of curves or bends along their respective lengths to form a deflected X-shape bridge and providing the benefit of better fitting around the spinous process of the spinal bone segments. More specifically, first curve 4501, second curve 4502, third curve 4503, fourth curve 4504, fifth curve 4505, and sixth curve 4506 along the first connector 3810 and second connector 3850 are included to provide clearance around any spinous process that might otherwise need to be removed in order to fit a bridge across the spinal bone segments. The curves or bends may be formed as a gradual, smooth surface or may be formed as a sharp and abrupt bend. Moreover, the first connector 4310 and/or the second connector 4350 may also incorporate an arced configuration so as to extend the Real-X cross connector 4300 outwardly along the axis A₄₃ and away from the spinal bone segments when the Real-X cross connector 4300 is installed in a patient.

With reference to FIGS. 43-44, the sphere 4410 of the second connector 4350 may be received by the spherical housing 4420 of the first connector 4310 which is complementary configured and positioned. In an alternative embodiment, the sphere 4410 and/or the spherical housing 4420 may be of any shape, substantially spherical or otherwise, that allows for rotation in three dimensions when the two components are received together. The sphere 4410 may snugly fit within the opening defined by the center sphere housing 4420, but still be capable of rotational movement for adjusting the position of the first connector 4310 and the second connector 4350 with respect to each other. Engaging the sphere 4410 with the spherical housing 4420 provides a spherical rotation joint for the Real-X cross connector 4300, thereby allowing the Real-X cross connector 4300 to be adjustable in three dimensions in order to fit varying spinal proportions of different patients. Not only can the first connector 4310 or the second connector 4350 rotate in relation to each other along the xy-plane, but the spherical joint enables rotation also along the z-axis, thus providing full three-dimensional rotation capabilities. The arms of the Real-X cross connector may thus be adjustably positioned both to accommodate not only the varying distances between a patient's spinal bone segments, but also may accommodate varying heights of the spinal bone segments by rotating the arms of the first connector 4310 and/or second connector 4350 along the z-axis. In an alternative embodiment, other shapes that permit rotation in three dimensions may be employed in place of the sphere 4410. The sphere 4410 may be formed with a rough or uneven surface, such as protruding or recessing concentric circles, for better making frictional contact with connecting components, as described above. The entire sphere 4410 may have the rough or uneven surface, or only a portion of the sphere 4410 may have the rough or uneven surface.

The spherical housing 4420 contains a plurality of ports 4560 for accommodating the connection of the sphere 4410 to its respective arms 4352 and 4354 when the sphere 4410 is positioned in the spherical housing 4420. The size and/or shape of the plurality of ports 4560 define the limits of the three dimensional rotation permitted by the first connector 4310 with respect to the second connector 4350. For example, ports 4560 that are narrow in width by taller in height would allow for a smaller respective range of rotational motion in the xy-plane, but a larger respective range of rotational motion along the z-axis due. The spherical housing 4420 also includes an interior threaded surface 4512 for mating with the set screw 4430, as discussed below for FIGS. 46A-B.

With reference to FIGS. 43-45B, FIG. 46A shows a set screw 4600 that may be the same or similar to the set screw 4430. The set screw 4600 may be non-cannulated as shown or, in an alternative embodiment, may be a cannulated screw. Upon rotating the first connector 4310 and/or the second connector 4350 into a desired or particular position, the first and second connectors 4310 and 4350 are then secured or locked in that position to prevent their movement after the installation in the patient is complete by the set screw 4430. The set screw 4600 includes a threaded portion 4612 disposed along an outer circumference for engaging the set screw 4600 with a connecting surface configured to receive such threading. For example, the set screw 4430, which may be set screw 4600, can engage the threaded portion 4612 with the interior threaded surface 4512 of the spherical housing 4420 in order to secure the first connector 4310 with the second connector 4350.

FIG. 46B shows a cross-section of the set screw 4600 to better illustrate its structural and functional features. A hollow portion 4620 at one end of the set screw 4600 provides a opening for the insertion of a screw driver or other mechanical component to facilitate the rotation of the screw into place via the engaging of the threaded portion 4612 with a receiving surface (e.g., the interior threaded surface 4512 of the spherical housing 4420 of the first connector 4310). The set screw 4600 may be cannulated or non-cannulated. A semi-spherical depression 4622 is disposed along a lower portion of the set screw 4600 and is configured to engage with a substantially spherical ball. The semi-spherical depression 4622 may have a rough or uneven surface for better making frictional contact with the substantially spherical ball (e.g. the sphere 4410) when the set screw 4600 is securely engaged. In one embodiment, the rough or uneven surface may be formed by a plurality of protruding or recessing concentric circles as previously discussed.

For example, when the set screw 4430 is the set screw 4600 and is not securely engaged with the interior threaded surface 4512 of the spherical housing 4420, the sphere 4410 of the second connector 4350 has minimal if any frictional contact with the semi-spherical depression 4622 of the set screw 4430 and is thus allowed to rotate in three dimensions as previously discussed to a desired position. Upon securely engaging the set screw 4430 with the threaded interior surface 4512 of the spherical housing 4420 containing the sphere 4410, the semi-spherical depression 4622 of the set screw 4430 accepts a portion of the sphere 4410 and makes frictional contact with the center sphere 4410 via the rough or uneven surface present on the semi-spherical depression 4622 and/or the center sphere 4410. This frictional contact maintains the first connector 4310 and the second connector 4350 in the desired position with respect to one another.

The discussion now turns to various dimensions or orientations of the Real-X cross connectors 3800, 4200, and/or 4300. The Real-X cross connectors 3800, 4200, and/or 4300 can be installed in a variety of configurations and locations along the spinal column of a patient. They may be installed across adjacent vertebrae of a patient's spinal column or may be installed to skip vertebrae. Advantageously, the Real-X cross connectors may be configured to accommodate a spinous process of a patient without requiring the removal of said spinous process. For example, the connecting rods 3801, 3802, 3803, and/or 3804 of the Real-X cross connector 3800 may be orientated at a desired angle via their spherical joints so as to avoid making contact with a non-removed spinous process of the patient. Similar accommodations may be made utilizing non-spherical connecting rods or the joint at the fulcrum of a Real-X cross connector. This flexibility during installation of the Real-X cross connectors 3800, 4200, and/or 4300 also allows for adaptable placement of the given cross connector even if the spinous process of the patient is removed.

The Real-X cross connectors 3800, 4200, and/or 4300 can be created in a variety of sizes depending upon their expected placement locations in a patient. For example, a Real-X cross connector for placement in the cervical (neck) region of a patient may be smaller than a Real-X cross connector for placement in the lumbar region of a patient. In one embodiment, a first connector 3810, 4210, or 4310 and a second connector 3850, 4250, or 4350 may be sized to span a distance between 20-60 mm for a cervical region of a patient, but may be sized to span a distance between 40-80 mm for a lumbar region of a patient. The Real-X cross connectors 3800, 4200, and/or 4300 may also be formed to curve or arc outwardly from the spinal cord of a patient and thus provide additional protection to the spine in the case of an impact to the back of the patient.

Turning our discussion now to FIG. 47, a perspective view of an alternative spinal bridge 4700 utilizing a spherical joint is shown. A first pedicle screw 4741, a second pedicle screw 4742, a third pedicle screw 4743, and a fourth pedicle screw 4744 each have a threaded shaft 4750 for their respective attachment to a spinal bone segment of a patient. A first connecting rod 4762 is connected between the first pedicle screw 4741 and the second pedicle screw 4742. Similarly, a second connecting rod 4764 is connected between the third pedicle screw 4743 and the fourth pedicle screw 4744. The spinal bridge 4700 mechanically links the first connecting rod 4762 and the second connecting rod 4764.

FIG. 48 shows a disassembled view of the bridge shown in FIG. 47 to better illustrate the component parts making up the spinal bridge 4700. A first clamping member 4810 has a first clamping element 4807 at a proximal end, a spherical housing 4812 at a distal end, and an extension element 4802 connected there between. The spherical housing 4812 may be the same or similar to the spherical housing 4420, as previously discussed for FIGS. 43-46B. Similarly, a second clamping member 4820 has a substantially spherical element 4806 at a proximal end, a clamping element 4805 at a distal end, and an extension element 4801 connected there between. The substantially spherical element 4806 may be the same or similar to the sphere 4511, as previously discussed for FIGS. 43-46B, and be formed with a rough or uneven surface (e.g. concentric circles). The spherical housing 4812 of the first clamping member 4810 is configured to receive the substantially spherical element 4805 of the second clamping member 4820. In one embodiment, the first clamping member 4810 may have a length of roughly 30 mm, measured from the center of the spherical housing 4812 to the end of the first clamping element 4807 and the second clamping member 4820 may have a length of roughly 30 mm measured from the center of the substantially spherical element 4806 to the end of the second clamping element 4805. Thus, a maximum total distance of roughly 60 mm may be obtained from the end of the first clamping element 4807 to the end of the second clamping element 4805 when the first clamping member and the second clamping member are engaged together and oriented within the same plane. An alternative embodiment may shorten or lengthen the respective clamping members in order to obtain a smaller or larger maximum total distance. An alternative embodiment may also utilize different connecting methods as previously described, for example the same or similar to the embodiments shown in FIGS. 1A-C, 2A-C, or with spherical joints or ends.

When the substantially spherical element 4805 is seated within the spherical housing 4812, the second clamping member 4820 is permitted to rotate in three dimensions with respect to the first clamping member 4810. The spherical housing 4812 contains a port 4860 for accommodating the extension element 4801 connected to the substantially spherical element 4806 when the substantially spherical element 4806 is positioned within the spherical housing 4812. The size and/or shape of the port 4860 may define the limits of the three dimensional rotation permitted by the first clamping member 4810 with respect to the second clamping member 4820. The spherical housing 4812 also includes an interior threaded surface 4814 for mating with a set screw 4830. The set screw 4830 may be the same or similar to the center screw 4600, previously discussed for FIG. 46. Upon rotating the first clamping member 4810 and/or the second clamping member 4820 into a desired or particular position, the first and second clamping members 4810 and 4820 are then secured or locked in that position to prevent their movement after the installation in the patient is complete by the set screw 4830. The set screw 4830 includes a threaded portion 4815 disposed along an outer circumference for engaging the set screw 4830 with the interior threaded surface 4814 of the spherical housing 4812. A semi-spherical depression 4850 receives and makes frictional contact with a portion of the substantially spherical element 4806 when the set screw 4830 is secured in position with the first clamping member 4810. The semi-spherical depression 4850 may be the same or similar to the semi-spherical depression 4622, as discussed for FIG. 46, and utilize the same or similar rough or uneven surface (e.g. concentric circles) to promote improved gripping capabilities.

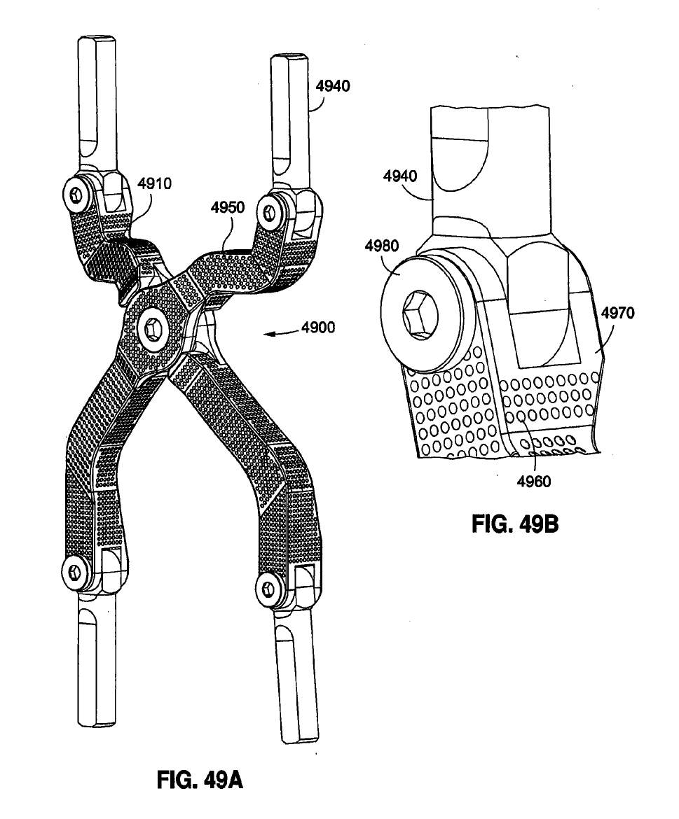

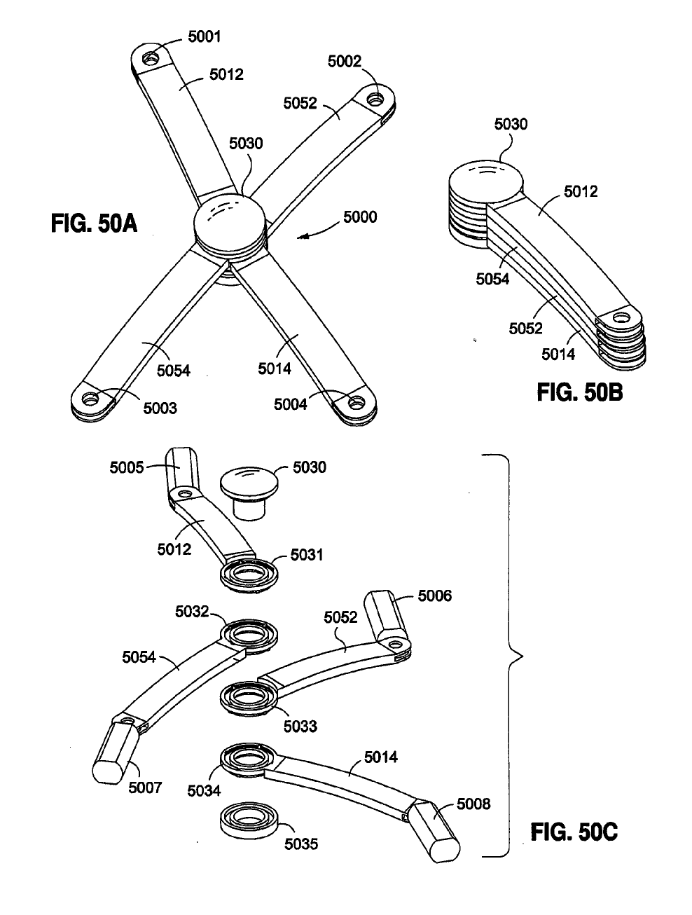

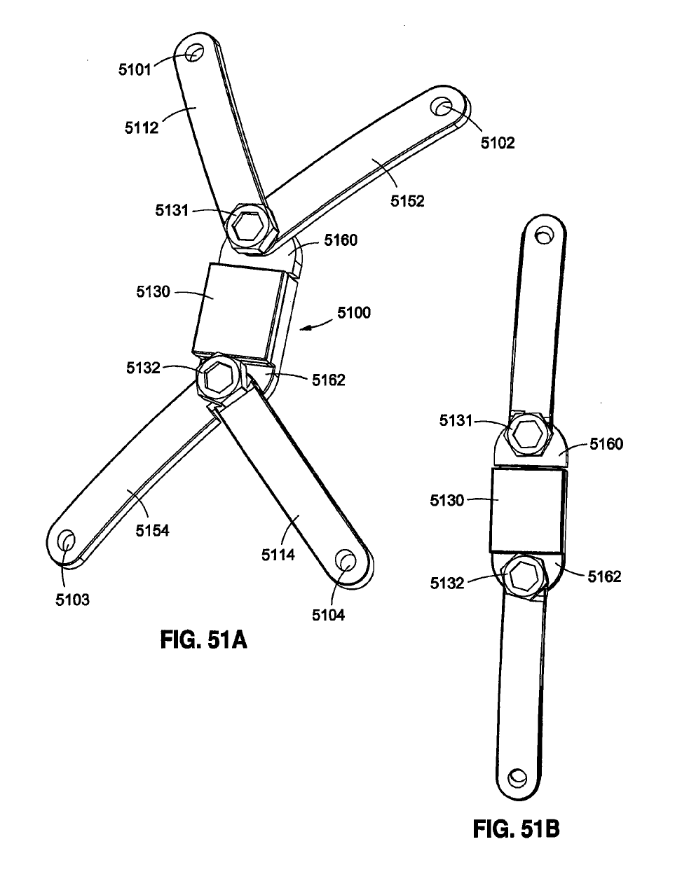

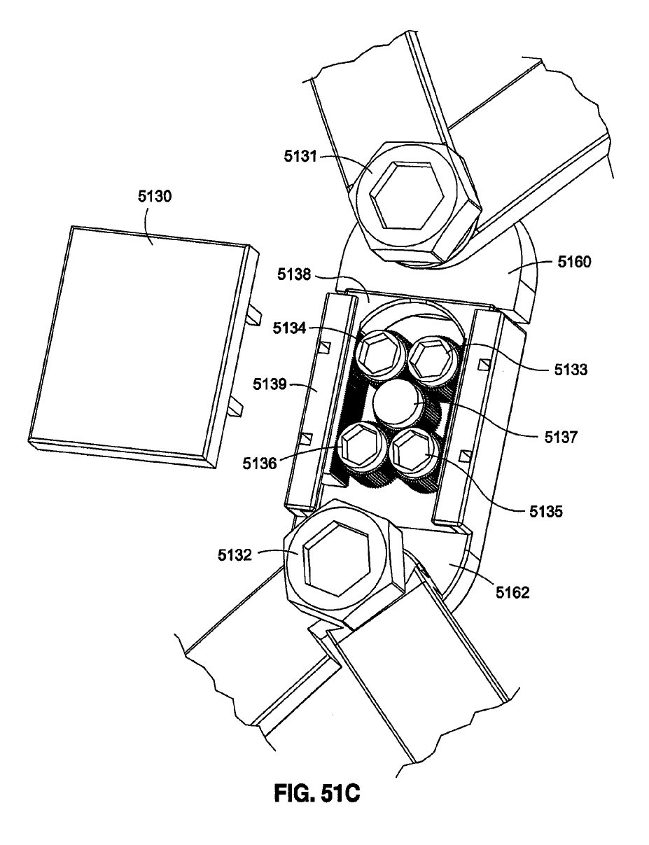

The discussion now turns to alternative embodiments of spinal cross connectors or spinal bridges incorporating dimples or designed for minimally invasive surgery. Dimpling the surface of spinal cross connectors or bridges can provide a surface for improved attachment of bone grafts and may be used upon the surface of a Real-X cross connector, the structural and functional features disclosed by FIGS. 49A-49B. Spinal hardware designed for minimally invasive surgery may be adapted for insertion into a patient through a smaller incision than commonly utilized for open surgery procedures. One embodiment designed for minimally invasive procedures is a collapsible spinal cross connector, the structural and functional features disclosed by FIGS. 50A-50C. A second embodiment designed for minimally invasive procedures is a partially collapsible spinal cross connector with adjustment gearing, the structural and functional features disclosed by FIGS. 51A-51C.

FIG. 49A shows a perspective view of a Real-X cross connector 4900 that incorporates dimples upon its surface for improved bonding with bone grafts. The Real-X cross connector 4900 has a first connector 4910 and a second connector 4950 coupled together and configured to extend across adjacent spinal segments of a patient. A connecting rod 4940 may be connected at the ends of each of the first connector 4910 and/or the second connector 4950 for coupling with a pedicle screw or other attachment mechanism for mounting the Real-X cross connector 4900 to the spinal segments of a patient. The exposed surfaces of the Real-X cross connector 4900 are covered with a dimpled surface, as discussed in greater detail below.

FIG. 49B shows a zoomed in perspective view of the Real-X cross connector 4900 and shows a plurality of recessed dimples 4960 disposed on the surface. The dimples 4960 may be positioned both upon the outwardly-facing surfaces of the first connector 4910 and the second connector 4950, and also upon any other exposed surface of the Real-X cross connector 4900 or its component parts (e.g. side-facing surface 4970). Although the dimples 4960 are shown as round depressions upon the surface, in an alternative embodiment the dimples 4960 can be of any shape and/or size so as to facilitate bonding with a bone graft. While bone grafts are commonly placed upon the bone segments of a patient, the bone grafts may also be smeared or placed across the Real-X cross connector 4900 and thus bond with the dimples 4960. Such a configuration may provide additional support and/or stability for coupling the Real-X cross connector 4900 with the spinal segments of the patient. The dimples 4960 may be disposed upon any or every exposed surface of the Real-X cross connector 4900, including the connecting rods 4940, the screw 4980 or any other exposed element. Dimpled surfaces may be utilized not only upon embodiments of Real-X cross connectors, but may also be incorporated upon any of the same or similar spinal connectors, bridges, or other components described or shown elsewhere in this application.

Turning next to spinal connectors designed for minimally invasive surgery, FIG. 50A shows a perspective view of a collapsible minimally invasive cross connector 5000. The cross connector 5000 has a first arm 5012, a second arm 5052, a third arm 5014, and a fourth arm 5054 rotatably connected together by a fulcrum member 5030. As discussed herein, the numerical terms, such as “first,” “second,” “third,” and “fourth” are relative terms such that they may be used interchangeably. Moreover, as discussed herein, the positioning terms, such as “top” and “bottom” are relative terms such that they may also be used interchangeably.

As seen in FIG. 50B, each of the first arm 5012, the second arm 5052, the third arm 5014, and the fourth arm 5054 are configured to rotate with respect to one another at the fulcrum member 5030. In an expanded configuration (see FIG. 50A), the arms may form a substantially X-shaped configuration for attachment across a patient's spinal bone segments. In a collapsed configuration (see FIG. 50B), the arms may form a stack on top of one another, substantially reducing the overall dimensions of the cross connector 5000. In the expanded configuration, the cross connector 5000 may act as a protective spinal bridge. However, open surgery is commonly needed for the installation of such a spinal bridge due to the overall larger shape and/or size of the bridge. In the collapsed configuration, however, a smaller incision in the patient may accommodate the reduced overall dimensions of the cross connector 5000, thus allowing the cross connector 5000 to be installed in a patient through a minimally invasive surgical procedure.

FIG. 50C, with reference to FIG. 50A, shows an exploded perspective view of the cross connector 5000 for better demonstrating its structural and functional characteristics. At one end of the first arm 5012 is a first opening 5001. The first opening 5001 provides an attachment location for connecting the first arm 5012 with a first connecting rod 5005. The first opening 5001 may have a circular shape and be configured to receive a screw (not shown) in order to permit rotation of the first connecting rod 5005 about the first opening 5001 before securing the first connecting rod 5005 in position with the screw. In an alternative embodiment, any connecting means may be used (e.g., a spherical joint) to connect the first arm 5012 to the first connecting rod 5005, or no connecting rod may be utilized. At the other end of the first arm 5012 is a first connecting ring 5031. The first connecting ring 5031 may be formed as a part of the first arm 5012 or may be a discrete component that is mechanically fastened to the first arm 5012. The first connecting ring 5031 is configured to accept a portion of the fulcrum member 5030, as discussed below.

At one end of the second arm 5052 is a second opening 5002. The second opening 5002 provides an attachment location for connecting the second arm 5052 with a second connecting rod 5006. The second opening 5002 may have a circular shape and be configured to receive a screw (not shown) in order to permit rotation of the second connecting rod 5006 about the second opening 5002 before securing the second connecting rod 5006 in position with the screw. In an alternative embodiment, any connecting means may be used (e.g., a spherical joint) to connect the second arm 5052 to the second connecting rod 5006, or no connecting rod may be utilized. At the other end of the second arm 5052 is a second connecting ring 5033. The second connecting ring 5033 may be formed as a part of the second arm 5052 or may be a discrete component that is mechanically fastened to the second arm 5052. The second connecting ring 5033 is configured to accept a portion of the fulcrum member 5030, as discussed below.

At one end of the third arm 5014 is a third opening 5004. The third opening 5004 provides an attachment location for connecting the third arm 5014 with a third connecting rod 5008. The third opening 5004 may have a circular shape and be configured to receive a screw (not shown) in order to permit rotation of the third connecting rod 5008 about the third opening 5004 before securing the third connecting rod 5008 in position with the screw. In an alternative embodiment, any connecting means may be used (e.g., a spherical joint) to connect the third arm 5014 to the third connecting rod 5008, or no connecting rod may be utilized. At the other end of the third arm 5014 is a third connecting ring 5034. The third connecting ring 5034 may be formed as a part of the third arm 5014 or may be a discrete component that is mechanically fastened to the third arm 5014. The third connecting ring 5034 is configured to accept a portion of the fulcrum member 5030, as discussed below.

At one end of the fourth arm 5054 is a fourth opening 5003. The fourth opening 5003 provides an attachment location for connecting the fourth arm 5054 with a fourth connecting rod 5007. The fourth opening 5003 may have a circular shape and be configured to receive a screw (not shown) in order to permit rotation of the fourth connecting rod 5007 about the fourth opening 5003 before securing the fourth connecting rod 5007 in position with the screw. In an alternative embodiment, any connecting means may be used (e.g., a spherical joint) to connect the fourth arm 5054 to the fourth connecting rod 5007, or no connecting rod may be utilized. At the other end of the fourth arm 5054 is a fourth connecting ring 5032. The fourth connecting ring 5032 may be formed as a part of the fourth arm 5054 or may be a discrete component that is mechanically fastened to the fourth arm 5054. The fourth connecting ring 5032 is configured to accept a portion of the fulcrum member 5030, as discussed below.

The fulcrum member 5030 may have a protruding element that is received by each of the first connecting ring 5031, the second connecting ring 5033, the third connecting ring 5034, and the fourth connecting ring 5032. An end cap 5035 engages with the protruding element of the fulcrum member 5030 and operates to secure the fulcrum member 5030 with each of the connecting rings (e.g., 5031, 5033, 5034, 5032) in order to maintain the cross connector 5000 as one unit. In one embodiment, each of the first connecting ring 5031, the second connecting ring 5033, the third connecting ring 5034, and the fourth connecting ring 5032 may be configured to accept a portion of an adjacent connecting ring for fitment purposes when stacked together. Each of the arms (e.g. 5012, 5052, 5014, 5054) are rotatable with respect to one another about the fulcrum member 5030. By rotating the arms so that they stack on top of or below one another, the collapsed configuration seen in FIG. 50B can be obtained. By rotating the arms so that they expand outwardly from one another, the expanded configuration seen in FIG. 50A can be obtained. Although the cross connector 5000 is shown with substantially straight arms, it is envisioned that various features of other embodiments described in this application (e.g., arms incorporating curvatures or bends) may be utilized in an alternative embodiment.

FIG. 51A shows a perspective view of a geared minimally invasive cross connector 5100. The cross connector 5100 includes a first arm 5112, a second arm 5152, a third arm 5114, and a fourth arm 5154. The first arm 5112 and the second arm 5152 are rotatably coupled together by a first screw 5131 at one end of each of the first arm 5112 and the second arm 5152. Similarly, the third arm 5114 and the fourth arm 5154 are rotatably coupled together by a second screw 5132 at one end of each of the third arm 5114 and the fourth arm 5154. As discussed herein, the numerical terms, such as “first,” “second,” “third,” and “fourth” are relative terms such that they may be used interchangeably. Moreover, as discussed herein, the positioning terms, such as “top” and “bottom” are relative terms such that they may also be used interchangeably.

The first screw 5131 is coupled to a first platform 5160 and the second screw 5132 is coupled to a second platform 5162. The first platform 5160 and the second platform 5162 are configured to engage with each other as discussed in greater detail herein. A cover 5130 may be positioned over a portion of the first platform 5160 and the second platform 5162 when they are engaged together to prevent bodily fluids or other particulates from interfering with the engagement of the first platform 5160 with the second platform 5162. Although the cross connector 5100 is shown with substantially straight arms, it is envisioned that various features of other embodiments described in this application (e.g., arms incorporating curvatures or bends) may be utilized in an alternative embodiment.

As seen in FIG. 51B, the first arm 5112 and the second arm 5152 are configured to rotate with respect to one another at the first screw 5131 so that they may be stacked on top of or below one another. Similarly, the third arm 5114, and the fourth arm 5154 are configured to rotate with respect to one another at the second screw 5132 so that they may be stacked on top of or below one another. In an expanded configuration (see FIG. 51A), the arms may form a substantially X-shaped configuration for attachment across a patient's spinal bone segments. Each arm may be positioned according to the spinal bone segments of a given patient and then secured in place by the tightening of either the first screw 5131 or the second screw 5132. In a collapsed configuration (see FIG. 51B), certain arms may stack upon one another, thereby substantially reducing the overall dimensions of the cross connector 5100. In the expanded configuration, the cross connector 5100 may act as a protective spinal bridge. Open surgery is commonly needed for the installation of a spinal bridge due to the overall shape and/or dimensions of the bridge, however, the reduced dimensions of the cross connector 5100 in the collapsed configuration may permit installation of the cross connector 5100 into a patient via a smaller incision, such as those used during minimally invasive surgical procedures.

FIG. 51C shows a zoomed perspective view of the cross connector 5100 for better demonstrating its structural and functional characteristics. The cover 5130 is shown removed from the first platform 5160 and the second platform 5162 so that the underlying engagement mechanism can be better viewed and described. The first platform 5160 is formed with or is connected to an engagement member 5138. The second platform 5162 is formed with or is connected to a pair of guiding elements 5139 configured to receive the engagement member 5138 of the first platform 5160. A plurality of gears, including a first gear 5133, a second gear 5134, a third gear 5135, and a fourth gear 5136 are connected to the second platform 5162 and positioned between the pair of guiding elements 5139. The first gear 5133, the second gear 5134, the third gear 5135, and the fourth gear 5136 each operate to engage or mesh with a toothed surface of the engagement member 5138 in order to adjust and/or hold the first platform 5160 in a specific position with respect to the second platform 5162.

When one of the first gear 5133, the second gear 5134, the third gear 5135, or the fourth gear 5136 is rotated, the engagement member 5138 of the first platform 5160 is translated or moves with respect to the second platform 5162 within the guiding elements 5139 due to its engagement with one or more of the gears. In this manner, each of the first gear 5133, the second gear 5134, the third gear 5135, and the fourth gear 5136 may cooperate to either extend or retract the first platform 5160 with respect to the second platform 5162. In an alternative embodiment, no guiding elements 5139 may be utilized.

A locking gear 5137 is positioned and configured to provide a mechanical connection between the first gear 5133, the second gear 5134, the third gear 5135, and the fourth gear 5136 such that, after any needed rotation of the first gear 5133, the second gear 5134, the third gear 5135, or the fourth gear 5136 to adjust the position of the first platform 5160 with respect to the second platform 5162, the adjusted position can be secured. By inserting the locking gear 5137 between the first gear 5133, the second gear 5134, the third gear 5135, and the fourth gear 5136, further rotation of those gears is prevented and the first platform 5160 is thus held in place with respect to the second platform 5162. The locking gear 5137 may be a separate component as shown or, in an alternative embodiment, may be formed as part of the cover 5130 such that placement of the cover 5130 over the first platform 5160 and second platform 5162 inserts the locking gear 5137 into position. Such a design allows for adjustment of the cross connector 5100 either during surgery or after its installation within a patient without having to remove and re-install the same or a different cross connector if it is subsequently determined that alternative sizing is needed. Moreover, through knowledge of the gear ratios employed by the cross connector 5100, precise rotation amounts can be determined in order to obtain specific extension or retraction distances.

Each of the first gear 5133, the second gear 5134, the third gear 5135, and/or the fourth gear 5136 may contain an opening configured to accept a device that can rotate the respective gear when inserted into the opening. The gears may be manually rotated through the use of a hand-held device, such as a screwdriver, such that rotation of the hand-held device at any of the first gear 5133, the second gear 5134, the third gear 5135, or the fourth gear 5135 causes translation of the first platform 5160 with respect to the second platform 5162. Alternatively, the rotation may be accomplished with or assisted by an automatic rotation device, for example one capable of rotating according to predetermined and/or precise rotational amounts. Adjustments can thus be made to the cross connector 5100 through a small incision in the patient that needs only be large enough to accommodate a portion of the device for rotating the respective gear. An alternative embodiment may utilize any number of gears. In still another embodiment, alternative engagement means may be employed in place of or in addition to gears, such that the first platform 5160 can be extended or retracted with respect to the second platform 5162.

Various structures and/or features have been disclosed throughout the illustrative embodiments presented above. It is expected that the structures and/or features for any of the embodiments so presented may be adapted and/or incorporated into the various other embodiments illustrated throughout. For example, components with spherical joints may be used in place of or in addition to components with non-spherical joints and vice versa to form a variety of alternative embodiments. In one example, the same or similar spherical joint described for FIGS. 43-46 may be applied to the RXB cross connector. In another example, the same of similar spherical end joints described for FIGS. 38-42 may be applied to the RXB cross connector.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

1. A cross connector for stabilizing and protecting one or more fixation levels of spinal bone segments, the cross connector comprising:
a plurality of arms including first, second, third, and fourth arms, the first arm and the third arm aligning along a first reference plane, the second arm and the fourth arm aligning along a second reference plane intersecting the first reference plane along a pivot axis;
a bottom plate centered along the pivot axis and substantially perpendicular to the first and second reference planes;
a pair of bottom side walls connected to the bottom plate so as to define a bottom valley having a plurality of bottom curved sections, each of the pair of bottom side walls connected to the first arm or the third arm to form a first contiguous arc segment;
a top plate snugly fitted within the bottom valley and engaging the bottom plate to provide a pivot point along the pivot axis; and
a pair of top side walls connected to the top plate so as to define a top valley having a plurality of top curved sections for embracing the bottom plate, each of the pair of top side walls connected to the second arm or the fourth arm to form a second contiguous arc segment.

2. The cross connector of claim 1, wherein:
the bottom plate includes a bottom convexly sloped edge for fitting with at least one of the plurality of top curved sections, and
the top plate includes a top convexly sloped edge for fitting with at least one of the plurality of bottom curved sections.

3. The cross connector of claim 1, wherein:
the bottom valley has a bottom contour substantially matching a top radial cross section of the top plate, and
the top valley has a top contour substantially matching a bottom radial cross section of the bottom plate.

4. The cross connector of claim 1, wherein:
the pair of bottom side walls provide a first geometric transition from the first arm and the third arm to the top plate and the bottom plate, and
the pair of top side walls provide a second geometric transition from the second arm and the fourth arm to the top plate and the bottom plate.

5. The cross connector of claim 1, wherein:
the pair of bottom side walls each includes a bottom concave section,
the pair of top side walls each includes a top concave section, and
the bottom concave sections cooperate with the top concave section to restrict a relative lateral movement between the bottom plate and the top plate.

6. The cross connector of claim 1, wherein:
the bottom valley has a bottom valley depth substantially equal to a top plate thickness of the top plate such that the pair of bottom side walls are flush with the top plate along the first reference plane, and
the top valley has a top valley depth substantially equal to a bottom plate thickness of the bottom plate such that the pair of top side walls are flush with the bottom plate along the second reference plane.

7. The cross connector of claim 1, wherein:
the first arm has a first arm extension distal to the bottom plate and curving away from the first reference plane,
the second arm has a second arm extension distal to the top plate and curving away from the second reference plane, and
the first arm extension and the second arm extension form an adjustable bracket surrounding a base segment of a spinous process.

8. The cross connector of claim 1, wherein:
the first arm has a first arm extension distal to the bottom plate and deviating from the first reference plane,
the second arm has a second arm extension distal to the top plate and deviating from the second reference plane, and
the first arm extension cooperates with the second arm extension to substantially conform with a contour of a spinous process.

9. A cross connector for stabilizing and protecting one or more fixation levels of spinal bone segments, the cross connector comprising:
a first connector including a first pair of arms and a first joint positioned between the first pair of arms, the first joint having:
a first platform having a first bell-shaped ridge connecting the first pair of arms to form a first contiguous arc along a first reference plane, the first bell-shaped ridge furnished with a first convex edge, and
a first bracket formed on the first platform, the first bracket having a first vertical concave contour substantially parallel to the first reference plane, and a first horizontal concave contour intersecting the first vertical concave contour and substantially perpendicular to the first reference plane;
a second connector including a second pair of arms and a second joint positioned between the second pair of arms, the second joint having a complementary configuration with respect to the first joint, the second joint connecting the second pair of arms to form a second contiguous arc along a second reference plane intersecting the first reference plane alone a center axis; and
a pivoting means for pivoting the first connector against the second connector along the center axis, thereby allowing a limited range of angular movement between the first pair of arms and the second pair of arms.

10. The cross connector of claim 9, wherein:
the first platform has a center region surrounding the center axis, the center region substantially wider than each of the first pair of arms, and
the first bell-shaped ridge provides a geometric transition from each of the first pair of arms to the center portion of the first platform.

11. The cross connector of claim 9, wherein the pivoting means substantially restricts a relative displacement between the first joint and the second joint.

12. The cross connector of claim 9, wherein:
at least on of the first pair of arms has a first arm extension distal to the first joint and curving away from the first reference plane,
at least on of the second pair of arms has a second arm extension distal to the top plate and curving away from the second reference plane, and
the first arm extension cooperates with the second arm extension form an adjustable bracket surrounding a base segment of a spinous process.

13. The cross connector of claim 9, wherein:
at least on of the first pair of arms has a first arm extension distal to the first joint and deviating from the first reference plane,
at least on of the second pair of arms has a second arm extension distal to the top plate and deviating from the second reference plane, and
the first arm extension cooperates with the second arm extension to substantially conform with a contour of a spinous process.

14. The cross connector of claim 9, wherein the complementary configuration of the second connector includes:
a second platform having a second bell-shaped ridge connecting the second pair of arms to form the second contiguous arc along the second reference plane, the second bell-shaped ridge complementarily fitted with the first horizontal concave contour, the second bell-shaped ridge furnished with a second convex edge complementarily fitted with the first vertical concave contour of the first bracket.

15. The cross connector of claim 14, wherein:
the second platform has a center region surrounding the center axis, the center region substantially wider than each of the second pair of arms, and
the second bell-shaped ridge provides a geometric transition from each of the second pair of arms to the center portion of the second platform.

16. The cross connector of claim 14, wherein the complementary configuration of the second connector includes a second bracket formed on the second platform, the second bracket having:
a second vertical concave contour substantially parallel to the second reference plane and complementarily fitted with the first bell-shaped ridge, and
a second horizontal concave contour intersecting the second vertical concave contour and substantially perpendicular to the second reference plane, the second horizontal concave contour complementarily fitted with the first convex ridge.

17. The cross connector of claim 15, wherein the first bracket cooperates with the second bracket to substantially restrict a lateral movement between the first platform and the second platform.

18. A cross connector for stabilizing and protecting one or more fixation levels of spinal bone segments, the cross connector comprising:
a first link including a first pair of arms, a lower platform, and two upper brackets, the lower platform having two bottom bow-shaped ridges connecting the first pair of arms to form a first contiguous arc along a first reference plane, the two bottom bow-shaped ridges each furnished with a bottom convex edge, the two upper brackets positioned between the two bottom bow-shaped ridges and each having an upper ventral concave surface facing away from one of the first pair of arms;
a second link including a second pair of arms, an upper platform, and two lower brackets, the upper platform having two upper bow-shaped ridges connecting the second pair of arms to form a second contiguous arc along a second reference plane intersecting the first reference plane alone a center axis, the two upper bow-shaped ridges each furnished with an upper convex edge, the two lower brackets positioned between the two upper bow-shaped ridges and each having a lower ventral concave surface facing away from one of the first pair of arms; and
a pivoting member connected to the lower and upper platforms, thereby pivoting the first link against the second link along the center axis while substantially restricting a lateral movement between the first link and the second link.

19. The cross connector of claim 18, wherein:
at least on of the first pair of arms has a first arm extension distal to the lower platform and curving away from the first reference plane,
at least on of the second pair of arms has a second arm extension distal to the top plate and curving away from the second reference plane, and
the first arm extension cooperates with the second arm extension form an adjustable bracket surrounding a base segment of a spinous process.

20. The cross connector of claim 18, wherein:
the upper ventral concave surfaces are configured to substantially redistribute a top stress directed to the upper convex edges of the upper bow-shaped ridges, and
the lower ventral concave surfaces are configured to substantially redistribute a bottom stress directed to the lower convex edges of the lower bow-shaped ridges.

21. A cross connector for stabilizing and protecting one or more fixation levels of spinal bone segments, the cross connector comprising:
a first elongated connector having a first arm and a second arm connected by a first joint element, the first arm defining an opening;
a second elongated connector including a third arm and a fourth arm connected by a second joint element, the second joint element configured to receive at least a portion of the first joint element; and
a first connecting rod having a substantially spherical portion, the substantially spherical portion of the first connecting rod configured to be received by the first opening of the first arm of the first elongated connector.

22. The cross connector of claim 21 wherein the substantially spherical portion of the first connecting rod is formed with a surface having a plurality of protruding concentric circles.

23. The cross connector of claim 21 further comprising a screw configured to engage with the first arm of the first elongated connector for coupling the first arm with the first connecting rod, the screw having a semi-spherical depression for receiving at least a portion of the substantially spherical portion of the first connecting rod.

24. The cross connector of claim 21 wherein the first joint element comprises a substantially spherical element and the second joint element comprises a housing configured to receive at least a portion of the substantially spherical element, the substantially spherical element capable of three dimensional rotation within the housing of the second joint element.

25. The cross connector of claim 24 wherein the substantially spherical element is formed with a surface having a plurality of protruding concentric circles.

26. The cross connector of claim 24 further comprising a screw configured to engage with the first elongated connector or the second elongated connector, the screw having a semi-spherical depression for receiving at least a portion of the substantially spherical element.

27. The cross connector of claim 21 wherein:
the first elongated connector, the second elongated connector, or the first connecting rod have a flexible construction, or
the first joint, the second joint, or the first opening are configured to be adjustable,
such that movement of the spinal bone segments is permitted after installation of the first elongated connector, the second elongated connector, and the first connecting rod.

28. The cross connector of claim 21 wherein:
the first elongated connector, the second elongated connector, and the first connecting rod comprise a rigid construction, and
the first joint, the second joint, and the first opening are configured to be securable,
such that movement of the spinal bone segments is prohibited after installation of the first elongated connector, the second elongated connector, and the first connecting rod in the patient.
